SEQUENCE LISTING (62 sequences)

SEQ ID NO: 1 - protein, 509 aa, synthetic construct
MSIISYIAFLLLIDSNLGIPIFVPSGRNISWQPVIQPFDYQCPIHGNLPNTMGLSATKLTIKSPSVFSTDKVSGWICHAAEWKTTCDYRWYGPQYITHSIHPISPTIDECRRIIQRIASGTDEDLGFPPQSCGWASVTTVSNTNYRVVPHSVHLEPYGGHWIDHEFNGGECREKVCEMKGNHSIWITEETVQHECAKHIEEVEGIMYGNVPRGDVMYANNFIIDRHHRVYRFGGSCQMKFCNKDGIKFARGDWVEKTAGTLTTIHDNVPKCVDGTLVSGHRPGLDLIDTVFNLENVVEYTLCEGTKRKINKQEKLTSVDLSYLAPRIGGFGSVFRVRNGTLERGSTTYIRIEVEGPIVDSLNGTDPRTNASRVFWDDWELDGNIYQGFNGVYKGKDGKIHIPLNMIESGIIDDELQHAFQADIIPHPHYDDDEIREDDIFFDNTGENGNPVDAVVEWVSGWGTSLKFFGMTLVALILIFLLIRCCVACTYLMKRSKRPATESHEMRSLV

SEQ ID NO: 2 - protein, 491 aa, synthetic construct
IPIFVPSGRNISWQPVIQPFDYQCPIHGNLPNTMGLSATKLTIKSPSVFSTDKVSGWICHAAEWKTTCDYRWYGPQYITHSIHPISPTIDECRRIIQRIASGTDEDLGFPPQSCGWASVTTVSNTNYRVVPHSVHLEPYGGHWIDHEFNGGECREKVCEMKGNHSIWITEETVQHECAKHIEEVEGIMYGNVPRGDVMYANNFIIDRHHRVYRFGGSCQMKFCNKDGIKFARGDWVEKTAGTLTTIHDNVPKCVDGTLVSGHRPGLDLIDTVFNLENVVEYTLCEGTKRKINKQEKLTSVDLSYLAPRIGGFGSVFRVRNGTLERGSTTYIRIEVEGPIVDSLNGTDPRTNASRVFWDDWELDGNIYQGFNGVYKGKDGKIHIPLNMIESGIIDDELQHAFQADIIPHPHYDDDEIREDDIFFDNTGENGNPVDAVVEWVSGWGTSLKFFGMTLVALILIFLLIRCCVACTYLMKRSKRPATESHEMRSLV

SEQ ID NO: 3 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 4 - protein, 519 aa, synthetic construct
MNTLIKILLIIIILREARSHIVLVPLDLGDWRTTEADQLDCPMHGDLSNQGTQAIELEYHTASWGLKNNIAGSLCVTAKWSITCDYRWYGSKYISTVIEYLPTTPEMCKEAKRASDRGESLAPHFPTENCGWNNVLTESQEFTTLTSHPVKLDAYSFILIDSMFEGGRCQSKECPVVFHQGMWIADQEAFGFCKDLDKHRGLLFKTGLRNSLGEIVRQEWNLNSVFQPEIGREKHFKGACKMSYCGNSGVRFSDREWFQLGTPSDNGIKKIIEGLPECGEDNLIHSHDTSNTLKELAEHVDEIALNAICLQEVRRARDTKAVSDWLLSMMSPFSEGIGKVYRIHKGRLESTVGFYRKVVLEGDGTPERLGVGLDKKPVSWDQFVVKTNDTRIQSMFNGNTVVNGKIKWVKNVLGAHILDEISALEFDVPLIPHPHLDGLKFNESHTISSHHPNGKGVNFVESVTHWAGGLWESIGSSAVIIVVLLICAFVAVKFCQRLIPSRRPPTRESSENVFMLRTV

SEQ ID NO: 5 - protein, 571 aa, synthetic construct
MKKTTEFTWPLKMYSSKFLLVTLSILVPRVISLFLPDYNPELFKPAVIHKLTCPNLSATSQINKYASMDTIYVNLGRPKPNFKVTVEGYLCTKIVLQTVCEAFLFSANEVNHIRKEAPIDKSECDSAIYRFIKGDVIDETFLPDYFSGYKRTVRQRIYIKVITHEVSYDPYLEKYVDSWFPGGATPYNYSTTIHDSTLWKMKGQKPPCTDFETINGEYTVLPDPKDLEHPLRFIWALGIKEKSYKNSCVVKFCGRPGILFEDGEWFSISTPESPTNYDVFITNLPACNSSASIKTRTVEQEMDAEFSNEYQLNLRFWCMEVLQGLTSGSILPQYKLAFLTQNSPGLGNVYKILNGTLMVTVGHYKKVKLNQLDSLHEIGADDAGNPVYVTDTQLTPGPIPDSYEWINGLVFSDKRWSIPLVDLVRNNLDNLLTMPLKLQAIEHSAASFMSASSSHLIEGDVITQYGGPTSKHDNWFVNYTTKSWEWLHWIVYIGVIVGGIFVLIILEKLGCYKIVYYLLKKLCQSKSSKKTKQPKVEYKKASSNQAQQIESRHEVIPQEDSVIVSLDRFDW

SEQ ID NO: 6 - protein, 610 aa, synthetic construct
MVFLSLSTIIFILSLRAVTCSNPLSYPNGILTNNSTHNHPLSDFYIFYENSSLTYTQFPVAPDCSSILDTRDEQYPTTVTLWKVDQESQAEWGLLLWQERIDTTCSWNFWGNYKGSIVSKSSVPLKDIPSGSARNGYWALSNDEVQEIDHVPYNLRYYCYWCRNEYPGSFYMRYVKKVRIIRNPDGSIKTPRGSWVHELDNLWGDQMRYLVIRRFGGESSCPLKIYDVRAGVLSKSRSNFILVSLPSLNLQFSVSLESTETKCSFGDKTYDIVQSMGGYLLSIDIGNANWRGPWDPTPQHPGRERRSIMEFPDQTSFRYNQFINYHSSPRHKRHDQEFEFPLSLKSSYDYAQFRYEQNFIIRQINKNFGLLQKSICDIQFSKWQNLSPPNLAMKIAHYVTGSIHSIGGVHHGSYSIQRTEKSITKVNLVFPIVIVHGMYKCQREPSKEVVWAEPVTGILFKSPIPTHFSLSSSWLPGVNGSSIVPLTGQILLPEITMDHLEVVQQVEAKMVKSMYTNVELFGSTEEFQRYQTQGITSDEQSNTVNPWIGLLIHGGVSIATGILVALLIPSILKLFRHIIEKGEASLEERLHLRETSRKEFVKVRGKPWGV

SEQ ID NO: 7 - protein, 522 aa, synthetic construct
MFPSIFCLMLLVMTSSVGQFILPIKEITPPRPINEHHLSCRLGPGALYPTTSSSVVVRSIHPDSNPVPIQGYLCSKKEYTLTCSENFLWMETVKKDSRSVPVTPSECVDAVNSWLQDKLDKDTQLTKDCGWMSTNTVKKEIFDIQGKHVRLNPYEMEYIDEILHRGKCSSKICQTKYADMLWVSEGDLNQTCPKMIGADVRMSLGDQKTPSSVILHSNYVPGVSLEGACRDFSFCGHRGMVLRTGHFVVVSSDFPSTIVKMYNLFQVCQKDLTIVSDNMNSEITSSKLDLMKIFLLDKCQQVVARIKGGLSVTRYDLGFLSPQISGVGSGYVASNRSVLVGQYKYMLVHSYVANCHSCRSCVINAVTDEGVIPVHLTPDQCPCANFNGCYLPNGIKVYKDKVYNPLLDIDEEFYHQMTEVELNPNYISHPKDVKETFLSLPATEEVEQHHGRTLDSVVGSLVPDFSFSIWPYFVGLGALVIIIIILWRRRSYKPPAEAPIRDFPVIYTPQSSFGPIAQIRWN

SEQ ID NO: 8 - protein, 240 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 9 - protein, 330 aa, Homo sapiens [sequence omitted]

SEQ ID NO: 10 - protein, 325 aa, synthetic construct
STKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - protein, 326 aa, Homo sapiens
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 7 aa, synthetic construct
GYPFTSY

SEQ ID NO: 13 - protein, 6 aa, synthetic construct
DPNSGD

SEQ ID NO: 14 - protein, 12 aa, synthetic construct
SPYYSNDNSMDY

SEQ ID NO: 15 - protein, 11 aa, synthetic construct
RASQSIGTSIH

SEQ ID NO: 16 - protein, 7 aa, synthetic construct
YASESIS

SEQ ID NO: 17 - protein, 9 aa, synthetic construct
QQSNSWPTT

SEQ ID NO: 18 - protein, 5 aa, synthetic construct
SYWIH

SEQ ID NO: 19 - protein, 17 aa, synthetic construct
RIDPNSGDTKYNEKFKN

SEQ ID NO: 20 - protein, 8 aa, synthetic construct
GYPFTSYW

SEQ ID NO: 21 - protein, 8 aa, synthetic construct
IDPNSGDT

SEQ ID NO: 22 - protein, 14 aa, synthetic construct
ARSPYYSNDNSMDY

SEQ ID NO: 23 - protein, 6 aa, synthetic construct
QSIGTS

SEQ ID NO: 24 - protein, 121 aa, synthetic construct
QVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSS

SEQ ID NO: 25 - protein, 108 aa, synthetic construct
DILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKR

SEQ ID NO: 26 - protein, 5 aa, synthetic construct
  REPEAT 1..5: note = The sequence may be present n times, wherein n is 1, 2, 3, 4, or 5
GGGGS

SEQ ID NO: 27 - protein, 20 aa, synthetic construct
GGGGSGGGGSGGGGSGGGGS

SEQ ID NO: 28 - protein, 249 aa, synthetic construct
DILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSS

SEQ ID NO: 29 - protein, 249 aa, synthetic construct
QVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKR

SEQ ID NO: 30 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 31 - protein, 210 aa, synthetic construct
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLYK

SEQ ID NO: 32 - protein, 7 aa, synthetic construct
RYTFTDY

SEQ ID NO: 33 - protein, 6 aa, synthetic construct
YPYNGG

SEQ ID NO: 34 - protein, 12 aa, synthetic construct
DHRYNEGVSFDY

SEQ ID NO: 35 - protein, 15 aa, synthetic construct
RASESVDGFGNSFMN

SEQ ID NO: 36 - protein, 7 aa, synthetic construct
LASNLES

SEQ ID NO: 37 - protein, 9 aa, synthetic construct
QQNNEDPYT

SEQ ID NO: 38 - protein, 5 aa, synthetic construct
DYNLH

SEQ ID NO: 39 - protein, 17 aa, synthetic construct
FIYPYNGGTGYNQKFKN

SEQ ID NO: 40 - protein, 8 aa, synthetic construct
RYTFTDYN

SEQ ID NO: 41 - protein, 8 aa, synthetic construct
IYPYNGGT

SEQ ID NO: 42 - protein, 14 aa, synthetic construct
ARDHRYNEGVSFDY

SEQ ID NO: 43 - protein, 10 aa, synthetic construct
ESVDGFGNSF

SEQ ID NO: 44 - protein, 121 aa, synthetic construct
EVQLQQSGPELVKPGASVKISCKASRYTFTDYNLHWVKLSHEKSLEWIGFIYPYNGGTGYNQKFKNKAKLTVDYSSSTAYMELRSLTSVDAAVYYCARDHRYNEGVSFDYWGQGTTLTVSS

SEQ ID NO: 45 - protein, 112 aa, synthetic construct
NIVLTQSPASLAVSLGQRATISCRASESVDGFGNSFMNWYQQKPGQSPKLLIYLASNLESGVPARFSGSGSRTDFTLTIDPVEADDAATYYCQQNNEDPYTFGGGTKLEIKR

SEQ ID NO: 46 - protein, 253 aa, synthetic construct
NIVLTQSPASLAVSLGQRATISCRASESVDGFGNSFMNWYQQKPGQSPKLLIYLASNLESGVPARFSGSGSRTDFTLTIDPVEADDAATYYCQQNNEDPYTFGGGTKLEIKRGGGGSGGGGSGGGGSGGGGSEVQLQQSGPELVKPGASVKISCKASRYTFTDYNLHWVKLSHEKSLEWIGFIYPYNGGTGYNQKFKNKAKLTVDYSSSTAYMELRSLTSVDAAVYYCARDHRYNEGVSFDYWGQGTTLTVSS

SEQ ID NO: 47 - protein, 253 aa, synthetic construct
EVQLQQSGPELVKPGASVKISCKASRYTFTDYNLHWVKLSHEKSLEWIGFIYPYNGGTGYNQKFKNKAKLTVDYSSSTAYMELRSLTSVDAAVYYCARDHRYNEGVSFDYWGQGTTLTVSSGGGGSGGGGSGGGGSGGGGSNIVLTQSPASLAVSLGQRATISCRASESVDGFGNSFMNWYQQKPGQSPKLLIYLASNLESGVPARFSGSGSRTDFTLTIDPVEADDAATYYCQQNNEDPYTFGGGTKLEIKR

SEQ ID NO: 48 - protein, 122 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 49 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 50 - protein, 15 aa, synthetic construct
GGGGSGGGGSGGGGS

SEQ ID NO: 51 - protein, 244 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 52 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 53 - protein, 110 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTS

SEQ ID NO: 54 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 55 - protein, 248 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGGSGGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 56 - protein, 248 aa, synthetic construct
SEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTS

SEQ ID NO: 57 - DNA, 4301 bp, synthetic construct
  source 1..4301: mol_type = other DNA
atgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacactaaggaagccttagataagatagaggaagaacaaaacaaaagtaagaaaaaggcacagcaagcagcagctgacacaggaaacaacagccaggtcagccaaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggttgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggttgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 58 - DNA, 1497 bp, synthetic construct
  source 1..1497: mol_type = other DNA
atgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacactaaggaagccttagataagatagaggaagaacaaaacaaaagtaagaaaaaggcacagcaagcagcagctgacacaggaaacaacagccaggtcagccaaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggttgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggttgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 59 - DNA, 619 bp, synthetic construct
  source 1..619: mol_type = other DNA
aaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggttgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggttggg

SEQ ID NO: 60 - DNA, 3011 bp, synthetic construct
  source 1..3011: mol_type = other DNA
tttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 61 - protein, 498 aa, synthetic construct
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPVQQIGGNYVHLPLSPRTLNAWVKLIEEKKFGAEVVPGFQALSEGCTPYDINQMLNCVGDHQAAMQIIRDIINEEVADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCKLVLKGLGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 62 - protein, 912 aa, synthetic construct
MNLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETFYVDGAANRETKLGKAGYVTDRGRQKVVPLTDTTNQKTELQAIHLALQDSGLEVNIVTDSQYALGIIQAQPDKSESELVSQIIEQLIKKEKVYLAWVPAHKGIGGNEQVDKLVSAGIRKVLFLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

(19) United States
(12) Patent Application Publication
(10) Pub. No.: US 2023/0293688 A1
(43) Pub. Date: Sep. 21, 2023

(54) FUSOGENIC RHABDOVIRUS GLYCOPROTEINS AND USES THEREOF

(71) Applicant: Interius BioTherapeutics, Inc., Philadelphia, PA (US)

(72) Inventors: Ronnie M. Russell, Philadelphia, PA (US); Bruce Schnepp, Philadelphia, PA (US); James I. Andorko, Philadelphia, PA (US); Michael Marinelli, Philadelphia, PA (US); Mohit Sachdeva, Philadelphia, PA (US); Debasish Boral, Philadelphia, PA (US); Jeffery Foss, Philadelphia, PA (US)

(21) Appl. No.: 18/167,444

(22) Filed: Feb. 10, 2023

Related U.S. Application Data
(60) Provisional application No. 63/267,870, filed on Feb. 11, 2022.

(51) Int. Cl. A61K 39/00; C07K 14/005; C07K 16/28; C12N 7/00

(52) U.S. Cl. CPC A61K 39/4631; C07K 14/005; C07K 16/2809; C07K 16/2803; C07K 16/2815; C12N 7/00; C07K 2319/30; C12N 2760/20022; C12N 2740/15051; C12N 2740/15021; C12N 2740/15071

(57) ABSTRACT

Provided for herein are fusogenic rhabdovirus glycoproteins and uses thereof, compositions comprising the same, and methods of using the same. Also provided for herein are pseudotyped viral particles comprising rhabdovirus glycoproteins as provided for herein and targeting moieties as provided for herein. Also provided are methods of generating and using the pseudotyped viral particles as provided for herein.

### CROSS REFERENCE TO RELATED APPLICATIONS

This application claims the benefit of U.S. Provisional Application Ser. No. 63/267,870 filed 11 Feb. 2022, which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on 3 Feb. 2023, is named INH-018US_SL.xml and is 72.3 kilobytes in size.

### FIELD

Embodiments provided herein relate to certain rhabdovirus glycoproteins and compositions comprising and methods of using the same.

### BACKGROUND

Rhabdoviridae is a family of negative-strand RNA viruses that can infect certain types of cells. The G structural protein can play a critical role during the initial steps of virus infection. First, it is responsible for virus attachment to specific receptors. After binding, virions enter the cell by a clathrin-mediated endocytic pathway. In the acidic environment of the endocytic vesicle, G triggers the fusion between the viral and endosomal membranes, which releases the genome in the cytosol for the subsequent steps of infection.

G proteins of various Rhabdoviridae can be used to pseudotype viruses to facilitate viral fusion across various cell types. For example, VSV-G protein has been widely used. However, the wild-type VSV-G protein has a wide tropism because of its binding to the low-density lipoprotein receptor (LDL-R) and other members of this receptor family serve as VSV receptors (Finkelshtein, D., Werman, A., Novick, D., Barak, S., and Rubinstein, M. (2013). LDL receptor and its family members serve as the cellular receptors for vesicular stomatitis virus. Proceedings of the National Academy of Sciences of the United States of America 110, 7306-7311, which is hereby incorporated by reference in its entirety). However, this broad tropism can inhibit the selective targeting of specific cell types. Therefore, there is a need, for modified alternative fusogenic proteins that can be used to pseudotype viruses that will be specifically fuse with cells that are desired. The present embodiments, fulfill these needs as well as others.

### BRIEF SUMMARY

In some embodiments, viral particles are provided. In some embodiments, the viral particles comprise a targeting moiety and a polypeptide comprising a viral structural protein comprising a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence of SEQ ID NO: 1 or SEQ ID NO: 2.

In some embodiments, pharmaceutical compositions comprising viral particles as provided herein are provided.

In some embodiments, methods of making the viral particle provided for herein are provided.

In some embodiments, methods of delivering a heterologous molecule to a target cell are provided. In some embodiments, the methods comprise contacting the cell with a viral particle as provided for herein.

In some embodiments, methods of delivering a heterologous molecule to a target cell in a subject are provided. In some embodiments, the methods comprise administering to the subject a viral particle as provided for herein, or a pharmaceutical composition comprising the same.

In some embodiments, methods of treating cancer in a subject are provided. In some embodiments, the methods comprise administering to the subject a viral particle as provided for herein, or a pharmaceutical composition comprising the same.

### BRIEF DESCRIPTION OF THE FIGURES

FIG. 1 illustrates the ability of various rhabdovirus G proteins to transduce SupT1 and PBMC cells alone or in combination with a CD7 binder.

FIG. 2A illustrates the ability of SVCV-G pseudotyped lentiviral particles harboring a CD7 binder attached to the viral surface through a Fc domain to transduce SupT1 cells as well as human and non-human primate PBMCs. FIG. 2B illustrates the transduction of cells in the absence of the CD7 binder. FIG. 2C illustrates the transduction of human and non-human primate PBMCs in terms of MOI calculated from SupT1 titration.

FIG. 3A illustrates the ability of SVCV-G pseudotyped lentiviral particles harboring a CD7 binder attached to the viral surface through a polypeptide stalk to transduce SupT1 cells as well as human and non-human primate PBMCs. FIG. 3B illustrates the transduction of cells in the absence of the CD7 binder. FIG. 3C illustrates the transduction of human and non-human primate PBMCs in terms of MOI calculated from SupT1 titration.

FIG. 4A illustrates the off target transduction of GFP in a panel of B-cell cell lines as compared to control SupT1 cells for SVCV-G pseudotyped lentiviral particles harboring a CD7 binder attached to the viral surface through a Fc domain. FIG. 4B illustrates the off target transduction of a CAR20-T2A-GFP construct in a panel of B-cell cell lines as compared to control SupT1 cells for SVCV-G pseudotyped lentiviral particles harboring a CD7 binder attached to the viral surface through a Fc domain.

FIG. 5A illustrates the off target transduction of GFP in a panel of B-cell cell lines as compared to control SupT1 cells for SVCV-G pseudotyped lentiviral particles harboring a CD7 binder attached to the viral surface through a polypeptide stalk. FIG. 5B illustrates the off target transduction of a CAR20-T2A-GFP construct in a panel of B-cell cell lines as compared to control SupT1 cells for SVCV-G pseudotyped lentiviral particles harboring a CD7 binder attached to the viral surface through a polypeptide stalk.

### DETAILED DESCRIPTION

Provided for herein are viral particles comprising a non-human rhabdovirus glycoprotein that can be used, for example, to pseudotype a virus, such as a lentivirus. In some embodiments, the pseudotyped viral-like particles are pseudotyped using viral glycoproteins of a Spring viremia of carp virus, such as the Spring viremia of carp virus G protein. In some embodiments, the viral particle comprises an amino acid sequence of SEQ ID NO: 1 or NO: 2. In some embodiments, the viral particle comprises an amino acid sequence that is that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to a sequence of SEQ ID NO: 1 or SEQ ID NO: 2

The pseudotyped viruses comprising the Spring viremia of carp virus G protein can be used in conjunction with a targeting moiety to facilitate the fusion of the pseudotyped virus with a specific cell or tissue based on the expression of the target on the cell or the tissue.

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments. Additionally, where a phrase recites “about x to y,” the term “about” modifies both x and y and can be used interchangeably with the phrase “about x to about y” unless context dictates differently.

As used herein, the term “individual” or “subject,” or “patient” used interchangeably, means any animal, including mammals, such as mice, rats, other rodents, rabbits, dogs, cats, swine, cattle, sheep, horses, or primates, such as humans.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists.”

As used herein, the term “contacting” means bringing together of two elements in an in vitro system or an in vivo system. For example, “contacting” virus or vector described herein with an individual or patient or cell includes the administration of the virus to an individual or patient, such as a human, as well as, for example, introducing a compound into a sample containing a cellular or purified preparation containing the cell.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or a linker sequences, such as the glycine/serine sequences described herein link the two domains together.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune cell activation compared to the immune cell activation detected in the absence of the composition. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the phrase “ex vivo” in reference to a cell being transduced, transfected or transformed ex vivo, refers to a cell being transduced, transfected or transformed outside of the subject, that is with the cells being removed from the subject before such cells are transduced, transfected or transformed.

“Identity” as used herein refers to the subunit sequence identity between two polymeric molecules such as between two nucleic acid or amino acid molecules, such as, between two polynucleotide or polypeptide molecules. When two amino acid sequences have the same residues at the same positions; e.g., if a position in each of two polypeptide molecules is occupied by an Arginine, then they are identical at that position. The identity or extent to which two amino acid or two nucleic acid sequences have the same residues at the same positions in an alignment is often expressed as a percentage. The identity between two amino acid or two nucleic acid sequences is a direct function of the number of matching or identical positions; e.g., if half of the positions in two sequences are identical, the two sequences are 50% identical; if 90% of the positions (e.g., 9 of 10), are matched or identical, the two amino acids sequences are 90% identical.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 60%, 80% or 85%, or 90%, 95% or even 99% identical at the amino acid level or nucleic acid to the sequence used for comparison. Other percentages of identity in reference to specific sequences are described herein.

Sequence identity can be measured/determined using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e3 and e100 indicating a closely related sequence. In some embodiments, sequence identity is determined by using BLAST with the default settings.

To the extent embodiments provided for herein, includes composition comprising various proteins, these proteins may, in some instances, comprise amino acid sequences that have sequence identity to the amino acid sequences disclosed herein. Therefore, in certain embodiments, depending on the particular sequence, the degree of sequence identity is greater than 50% (e.g. 60%, 70%, 75%, 80%, 85%, 90%, 91%, 92%, 93%, 94%, 95%, 96%, 97%, 98%, 99% or more) to the SEQ ID NOs disclosed herein. In addition to these percentages, other percentages of identity are provided for herein. Identity between polypeptides can be determined by the Smith-Waterman homology search algorithm as implemented in the MPSRCH program (Oxford Molecular), using an affine gap search with parameters gap open penalty—12 and gap extension penalty=1.

These proteins may, compared to the disclosed proteins, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, Substitution of single amino acids within these families does not have a major effect on the biological activity. The proteins may have one or more (e.g. 1, 2, 3, 4, 5, 6, 7, 8, 9, 10, etc.) single amino acid deletions relative to the disclosed protein sequences. The proteins may also include one or more (e.g. 1, 2, 3, 4, 5, 6, 7, 8, 9, 10, etc.) insertions (e.g. each of 1, 2, 3, 4 or 5 amino acids) relative to the disclosed protein sequences.

As used herein, the phrase “in vivo” in reference to a cell being transduced, transfected or transformed in vivo, refers to a cell being transduced, transfected or transformed in the subject without the cells being removed from the subject before such cells are transduced, transfected or transformed.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family that is able to infect non-dividing cells. Non-limiting examples of lentiviruses are HIV, SIV, and FIV. Vectors or viral-like particles derived from lentiviruses can be used to transduce cells and deliver genes or other molecules and have them expressed in a cell either in vitro (ex-vivo) or in vivo.

By the term “modified” as used herein, is meant a changed state or structure of a molecule or cell as provided herein. Molecules may be modified in many ways, including chemically, structurally, and functionally, such as mutations, substitutions, insertions, or deletions (e.g. internal deletions truncations). Cells may be modified through the introduction of nucleic acids or the expression of heterologous proteins.

By the term “modulating,” as used herein, is meant mediating an increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as, a human.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides. It will be understood that when a nucleotide sequence is represented by a DNA sequence (i.e., A, T, C, G), this also provides the corresponding RNA sequence (i.e., A, U, C, G) in which “U” replaces “T.”

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, the terms “nucleic acids” and “polynucleotides” as used herein are interchangeable. As used herein polynucleotides include, but are not limited to, all nucleic acid sequences which are obtained by any methods available in the art, including, without limitation, recombinant methods, i.e., the cloning of nucleic acid sequences from a recombinant library or a cell genome, using cloning technology and PCR, and the like, and by synthetic means.

As used herein, the terms “peptide,” “polypeptide,” and “protein” are used interchangeably, and refer to a compound comprised of a plurality of amino acid residues covalently linked by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “pseudotyped” or “pseudotyped viral particle”, as used herein, refers to a viral particle bearing glycoproteins derived from other viruses having envelopes or a viral vector encoding envelope glycoproteins from a virus that is different from the parental virus. The host range of the vector particles can thus be expanded or altered depending on the type of cell surface receptor used by the glycoprotein. For example, a virus can be pseudotyped with a Spring viremia of carp virus G protein as provided for herein.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. In some embodiments, the targeting moieties described herein that can be used to target the viral particles comprising the Spring viremia of carp virus G protein can specifically bind to their target.

The term “subject” includes living organisms, including those in which an immune response can be elicited (e.g., mammals). A “subject” or “patient,” as used therein, may be a human or non-human mammal. Non-human mammals include, for example, livestock and pets, such as ovine, bovine, porcine, canine, non-human primates, feline and murine mammals. In some embodiments, the subject is human.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into a cell. A “transfected” or “transformed” or “transduced” cell is one which has been transfected, transformed or transduced with exogenous nucleic acid. The cell includes the primary subject cell and its progeny. In some embodiments, the transfection, transformation, or transduction is performed or occurs in vivo.

To “treat” a disease as the term is used herein, means to reduce the frequency or severity of at least one sign or symptom of a disease or disorder experienced by a subject.

A “vector” is a composition of matter which comprises an isolated nucleic acid encoding a protein or a peptide. Numerous vectors are known in the art including, but not limited to, linear polynucleotides, plasmids, DNA, and RNA. Examples of viral vectors include, but are not limited to, Sendai viral vectors, adenoviral vectors, adeno-associated virus vectors, retroviral vectors, lentiviral vectors, and the like.

A “carrier” or “delivery vehicle” includes viral particles, viruses, polylysine compounds, and liposomes, which facilitate transfer of nucleic acid into cells. A carrier or delivery vehicle can also be used to deliver a protein or peptide to a cell.

Ranges: throughout this disclosure, various aspects of the embodiments can be presented in a range format. It should be understood that the description in range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Unless otherwise explicitly stated to the contrary, a range that is disclosed also includes the endpoints of the range.

Without being bound to any particular theory, the embodiments provided for herein have been found to show that a Spring viremia of carp virus G protein can be used to pseudotype a virus and transduce a cell when the virus comprises a targeting moiety. The Spring viremia of carp virus G protein as provided can be used, in some embodiments, to transduce a target cell and deliver a heterologous molecule to the targeted cells.

In some embodiments, a Spring viremia of carp virus G protein is provided that comprises SEQ ID NO: 1. SEQ ID NO: 1 is the full length protein and SEQ ID NO: 2 is the ectodomain of the Spring viremia of carp virus G protein that has the N-terminal signal peptide removed. Accordingly, in some embodiments, the protein comprises an amino acid sequence of SEQ ID NO: 2.

The Spring viremia of carp virus G protein can be used, for example, to pseudotype a virus, such as, but not limited to a lentivirus. Accordingly, in some embodiments, a viral particle comprising a Spring viremia of carp virus G protein as provided herein are provided. In some embodiments, the viral particle comprises a Spring viremia of carp virus G protein comprising SEQ ID NO: 1 or SEQ ID NO: 2 or sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to a sequence of SEQ ID NO: 1 or SEQ ID NO: 2. In some embodiments, the viral particle comprises a Spring viremia of carp virus G protein comprising an amino acid sequence having at least 80% identity to SEQ ID NO: 1 or SEQ ID NO: 2. In some embodiments, the viral particle comprises a Spring viremia of carp virus G protein comprising an amino acid sequence having at least 85% identity to SEQ ID NO: 1 or SEQ ID NO: 2. In some embodiments, the viral particle comprises a Spring viremia of carp virus G protein comprising an amino acid sequence having at least 90% identity to SEQ ID NO: 1 or SEQ ID NO: 2. In some embodiments, the viral particle comprises a Spring viremia of carp virus G protein comprising an amino acid sequence having at least 95% identity to SEQ ID NO: 1 or SEQ ID NO: 2. In some embodiments, the viral particle comprises a Spring viremia of carp virus G protein comprising an amino acid sequence having at least 98% identity to SEQ ID NO: 1 or SEQ ID NO: 2. In some embodiments, the viral particle comprises a Spring viremia of carp virus G protein comprising an amino acid sequence of SEQ ID NO: 1 or SEQ ID NO: 2.

In some embodiments, the viral particle comprises a targeting moiety. The targeting moiety can be used to target the viral particle comprising the Spring viremia of carp virus G protein to a cell that expresses the target to which the targeting moiety binds to. In some embodiments, the targeting moiety is an antibody, a scFv antibody, an antigen binding domain, an ankyrin repeat (e.g., DARPIN), a VHH domain antibody, a nanobody, single domain antibody, a FN3 domain, or any combination thereof. The targeting moiety may be attached to the viral surface through any appropriate polypeptide sequence. In some embodiments, the targeting moiety is attached to the viral surface via a polypeptide stalk. In some embodiments, the polypeptide stalk comprises a transmembrane domain. In some embodiments, the transmembrane domain comprises a CD8 and/or CD28 transmembrane domain. In some embodiments, the targeting moiety is attached to the viral surface through an IgG Fc stalk. In some embodiments, the IgG Fc stalk comprises a transmembrane domain. In some embodiments, the transmembrane domain comprises a CD8 and/or CD28 transmembrane domain. In some embodiments, the targeting moiety is attached (fused or linked) an envelope glycoprotein G or H of a virus of the Paramyxoviridae family, such as a morbillivirus, such as Measles virus, or a henipavirus, such as Nipah virus, Cedar virus, or Hendra virus. In some embodiments, the targeting moiety can be attached (fused or linked) to a glycoprotein of a virus of the Rhabdoviridae family, such as a vesicular stomatitis New Jersey virus, a vesicular stomatitis Indiana virus, a vesicular stomatitis Alagoas virus, a vesicular stromatitis Maraba virus, a vesicular stomatitis Carajas virus, Parainfluenza virus, Spodoptera frugiperda rhabdovirus isolate Sf G, Drosophila obscura sigmavirus 10A, Wuhan insect virus 7, Perch virus, or Spring viremia of carp virus. In some embodiments, the Spring viremia of carp virus protein is Spring viremia of carp virus G protein, such as those provided for herein. In some embodiments, the targeting moiety is attached to a glycoprotein of a virus of the Filoviridae family, such as Ebola virus or a glycoprotein of a virus of the Arenaviridae family, such as Machupo virus.

In some embodiments, the targeting moiety is selected from the group including, but not limited to, an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof. In some embodiments, the targeting moiety is a scFv. In some embodiments, the targeting moiety is a single domain antibody. In some embodiments, the targeting moiety is a VHH.

In some embodiments, the targeting moiety is selected from the group consisting of Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1.

In some embodiments, the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsg1); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-1a); Carbonic anhydrase IX (CA1X); Carcinoembryonic antigen (CEA); CCCTC-Binding Factor (Zinc Finger Protein)-Like (BORIS or Brother of the Regulator of lmprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLVl-Tax; Human papilloma virus E6 (HPV E6); Human papilloma virus E7 (HPV E7); Human Telomerase reverse transcriptase (hTERT); IgE; IL13Ra2; ILl lRa; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-1 (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAP1; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1). In some embodiments, the targeting moiety binds to CD7. In some embodiments, the targeting moiety binds to CD8.

In some embodiments, the targeting moiety binds to a target that is present on a cell, such as an immune cell. In some embodiments, the cell is an immune cell, such as, but not limited to, T cell, B cell; NK cell, dendritic cell, neutrophils, macrophages, a cancer cell; or, for example, CD3+ T cell; CD4+ T cell; CD7+ T cell, CD8+ T cell; CD19+ B cell; CD19+ cancer cell; CD20+ B cell; CD20+ cancer cell; CD30+ lung epithelial cell; CD34+ haematopoietic stem cell; CD105+ endothelial cell; CD105+ haematopoietic stem cell; CD117+ haematopoietic stem cell; CD133+ cancer cell; EpCAM+ cancer cell; GluA2+ neuron; GluA4+ neuron; Haematopoietic stem cell; Hepatocyte; Her2/Neu+ cancer cell; NKG2D+ natural killer cell; SLC1A3+ astrocyte; SLC7A10+ adipocyte. In some embodiments, the cell is a T cell. In some embodiments, the cell is a B cell. In some embodiments, the cell is a CD7+ T cell and/or CD8+ T cell.

In some embodiments, the targeting moiety (a polypeptide) can bind to CD7.

In some embodiments, the polypeptide binds to CD7. In some embodiments, the polypeptide that binds to CD7 is an antibody which binds to non-human primate CD7. In some embodiments, the polypeptide that binds to CD7 is an antibody which binds to human CD7. The sequence of human CD7 (UniProtKB P09564) is as follows (SEQ ID NO: 8):

(SEQ ID NO: 8)
MAGPPRLLLLPLLLALARGLPGALAAQEVQQSPHCT
TVPVGASVNITCSTSGGLRGIYLRQLGPQPQDIIYY
EDGVVPTTDRRFRGRIDFSGSQDNLTITMHRLQLSD
IGTYTCQAITEVNVYGSGTLVLVTEEQSQGWHRCSD
APPRASALPAPPTGSALPDPQTASALPDPPAASALP
AALAVISFLLGLGLGVACVLARTQIKKLCSWRDKNS
AACVVYEDMSHSRCNTLSSPNQYQ

In some embodiments, the CD7 antibody comprises a Fc region. The Fc region can be linked to the heavy or light chain of the antibody. The Fc region may be fused directly to the heavy or light chain of the antibody or may be fused indirectly to the heavy or light chain of the antibody via, for example, a peptide linker as provided for herein. In some embodiments, the Fc region is an IgG Fc. In some embodiments, the IgG is selected from IgG1, IgG2, IgG3, or IgG4. In some embodiments, the IgG fc is IgG1 Fc. In some embodiments, the antibody comprises an Fc constant region of SEQ ID NO: 9 as set forth below:

(SEQ ID NO: 9)
ASTKGPSVFPLAPSSKSTSGGTAALGCLVKDYFPEP
VTVSWNSGALTSGVHTFPAVLQSSGLYSLSSVVTVP
SSSLGTQTYICNVNHKPSNTKVDKKVEPKSCDKTHT
CPPCPAPELLGGPSVFLFPPKPKDTLMISRTPEVTC
VVVDVSHEDPEVKFNWYVDGVEVHNAKTKPREEQYN
STYRVVSVLTVLHQDWLNGKEYKCKVSNKALPAPIE
KTISKAKGQPREPQVYTLPPSRDELTKNQVSLTCLV
KGFYPSDIAVEWESNGQPENNYKTTPPVLDSDGSFF
LYSKLTVDKSRWQQGNVFSCSVMHEALHNHYTQKSL
SLSPGK

In some embodiments, the IgG fc is IgG2 Fc. In some embodiments, the antibody comprises an Fc constant region of SEQ ID NO: 10 as set forth below:

(SEQ ID NO: 10)
STKGPSVFPLAPCSRSTSESTAALGCLVKDYFPEPV
TVSWNSGALTSGVHTFPAVLQSS
GLYSLSSVVTVPSSSLGTQTYTCNVDHKPSNTKVDK
TVERKCCVECPPCPAPPVAGPSVFLFPPKPKDTLMI
SRTPEVTCVVVDVSHEDPEVQFNWYVDGVEVHNAKT
KPREEQFNSTFRVVSVLTVVHQDWLNGKEYKCKVSN
KGLPAPIEKTISKTKGQPREPQVYTLPPSREEMTKN
QVSLTCLVKGFYPSDIAVEWESNGQPENNYKTTPPM
LDSDGSFFLYSKLTVDKSRWQQGNVFSCSVMHEALH
NHYTQKSLSLSPGK

In some embodiments, the IgG fc is IgG4 Fc. In some embodiments, the antibody comprises an Fc constant region of SEQ ID NO: 11 as set forth below:

(SEQ ID NO: 11)
STKGPSVFPLAPCSRSTSESTAALGCLVKDYFPEPV
TVSWNSGALTSGVHTFPAVLQSSGLYSLSSVVTVPS
SSLGTKTYTCNVDHKPSNTKVDKRVESKYGPPCPSC
PAPEFLGGPSVFLFPPKPKDTLMISRTPEVTCVVVD
VSQEDPEVQFNWYVDGVEVHNAKTKPREEQFNSTYR
VVSVLTVLHQDWLNGKEYKCKVSNKGLPSSIEKTIS
KAKGQPREPQVYTLPPSQEEMTKNQVSLTCLVKGFY
PSDIAVEWESNGQPENNYKTTPPVLDSDGSFFLYSR
LTVDKSRWQEGNVFSCSVMHEALHNHYTQKSLSLSL
GK

In some embodiments, the IgG Fc comprises an amino acid sequence having at least 80% identity to SEQ ID NO: 9, SEQ ID NO: 10, or SEQ ID NO: 11. In some embodiments, the IgG Fc comprises an amino acid sequence having at least 85% identity to SEQ ID NO: 9, SEQ ID NO: 10, or SEQ ID NO: 11. In some embodiments, the IgG Fc comprises an amino acid sequence having at least 90% identity to SEQ ID NO: 9, SEQ ID NO: 10, or SEQ ID NO: 11. In some embodiments, the IgG Fc comprises an amino acid sequence having at least 95% identity to SEQ ID NO: 9, SEQ ID NO: 10, or SEQ ID NO: 11. In some embodiments, the IgG Fc comprises an amino acid sequence having at least 98% identity to SEQ ID NO: 9, SEQ ID NO: 10, or SEQ ID NO: 11. In some embodiments, the IgG Fc comprises an amino acid sequence of SEQ ID NO: 9, SEQ ID NO: 10, or SEQ ID NO: 11.

In some embodiments, polypeptides (e.g. CD7-binding polypeptide) are provided herein. In some embodiments, antibodies (e.g. an anti-CD7 antibody) are provided herein. In some embodiments, the antibody is a recombinant antibody that binds to CD7. In some embodiments, the CD7 protein is a human CD7 protein. In some embodiments, the CD7 protein is a non-human CD7 protein (e.g., mouse, rat, pig, dog, non-human primate). As used herein, the term “recombinant antibody” refers to an antibody that is not naturally occurring. In some embodiments, the term “recombinant antibody” refers to an antibody that is not isolated from a human subject.

In some embodiments, the CD7 antibody does not comprise a Fc region. In some embodiments, the CD7 antibody comprises a polypeptide stalk as provided for herein. In some embodiments, the CD7 antibody comprises a polypeptide stalk comprising a transmembrane domain as provided for herein.

In some embodiments, an antibody, or antigen binding fragment thereof is provided, wherein the antibody or antibody fragment comprises a peptide selected from the following table, which illustrate the CDRs based on Chothia numbering.

Chothia CDRs

| Ab ID No | HCDR1 | HCDR2 | HCDR3 | LCDR1 | LCDR2 | LCDR3 |
|---|---|---|---|---|---|---|
| CD7AB1 | GYPF | DPNS | SPYYS | RASQ | YAS | QQSNS |
| | TSY | GD | NDNSM | SIGT | ESIS | WPTT |
| | (SEQ | (SEQ | DY | SIH | (SEQ | (SEQ |
| | ID | ID | (SEQ | (SEQ | ID | ID |
| | NO: | NO: | ID | ID | NO: | NO: |
| | 12) | 13) | NO: | NO: | 16) | 17) |
| | | | 14) | 15) | | |

In some embodiments, an antibody, or antigen binding fragment thereof is provided, wherein the antibody or antibody fragment comprises a peptide selected from the following table, which illustrate the CDRs based on Kabat numbering.

Kabat CDRS

| Ab ID | | | | | | |
|---|---|---|---|---|---|---|
| No | HCDR1 | HCDR2 | HCDR3 | LCDR1 | LCDR2 | LCDR3 |
| CD7AB1 | SYWIH | RIDPN | SPYYS | RASQ | YASE | QQSNS |
| | (SEQ | SGDTK | NDNSM | SIGT | SIS | WPTT |
| | ID | YNEKF | DY | SIH | (SEQ | (SEQ |
| | NO: | KN | (SEQ | (SEQ | ID | ID |
| | 18) | (SEQ ID | ID | ID | NO: | NO: |
| | | NO: 19) | NO: | NO: | 16) | 17) |
| | | | 14) | 15) | | |

In some embodiments, an antibody, or antigen binding fragment thereof is provided, wherein the antibody or antibody fragment comprises a peptide selected from the following table, which illustrate the CDRs based on IMGT numbering.

IMGT CDRS

| Ab ID | | | | | | |
|---|---|---|---|---|---|---|
| No | HCDR1 | HCDR2 | HCDR3 | LCDR1 | LCDR2 | LCDR3 |
| CD7AB1 | GYPF | IDPN | ARSPY | QSIG | YA | QQSNS |
| | TSYW | SGDT | YSNDN | TS | | WPTT |
| | (SEQ | (SEQ | SMDY | (SEQ | | (SEQ |
| | ID | ID | (SEQ | ID | | ID |
| | NO: | NO: | ID | NO: | | NO: |
| | 20) | 21) | NO: | 23) | | 17) |
| | | | 22) | | | |

In some embodiments, a polypeptide, an antibody, or antibody binding fragment thereof, comprises a heavy or light chain CDR as provided in the tables above. In some embodiments, a polypeptide, an antibody, or antibody binding fragment thereof, comprises a heavy or light chain CDR as provided in the tables above and binds to non-human primate CD7. In some embodiments, a polypeptide, an antibody, or antibody binding fragment thereof, comprises a heavy or light chain CDR as provided in the tables above and binds to human CD7. In some embodiments, a polypeptide, an antibody, or antibody binding fragment thereof, comprises a light chain CDR having a sequence selected from SEQ ID NO: 15-17. In some embodiments, a polypeptide, an antibody, or antibody binding fragment thereof, comprises a light chain CDR having a sequence of SEQ ID NO: 15. In some embodiments, a polypeptide, an antibody, or antibody binding fragment thereof, comprises a light chain CDR having a sequence of SEQ ID NO: 16. In some embodiments, a polypeptide, an antibody, or antibody binding fragment thereof, comprises a light chain CDR having a sequence of SEQ ID NO: 17. In some embodiments, a polypeptide, an antibody, or antibody binding fragment thereof, comprises a heavy chain CDR having a sequence selected from SEQ ID NO: 12-14. In some embodiments, a polypeptide, an antibody, or antibody binding fragment thereof, comprises a heavy chain CDR having a sequence of SEQ ID NO: 12. In some embodiments, a polypeptide, an antibody, or antibody binding fragment thereof, comprises a heavy chain CDR having a sequence of SEQ ID NO: 13. In some embodiments, a polypeptide, an antibody, or antibody binding fragment thereof, comprises a heavy chain CDR having a sequence of SEQ ID NO: 14. The CDRs referenced in the embodiments throughout the present specification can be interchanged with the CDRs that are characterized by different formats, such as Kabat and IMGT.

In some embodiments, a polypeptide, an antibody, or antibody binding fragment thereof, comprises a light chain variable region having a LCDR1, a LCDR2, and a LCDR3, wherein the LCDR1 has a sequence of SEQ ID NO: 15, the LCDR2 has a sequence of SEQ ID NO: 16, and the LCDR3 has a sequence of SEQ ID NO: 17.

In some embodiments, a polypeptide, an antibody, or antibody binding fragment thereof, comprises a light chain variable region having a LCDR1, a LCDR2, and a LCDR3, wherein the LCDR1 has a sequence of SEQ ID NO: 23, the LCDR2 has a sequence of YA, and the LCDR3 has a sequence of SEQ ID NO: 17.

In some embodiments, a polypeptide, an antibody, or antibody binding fragment thereof, comprises a heavy chain variable region having a HCDR1, a HCDR2, and a HCDR3, wherein the HCDR1 has a sequence of SEQ ID NO: 12, the HCDR2 has a sequence of SEQ ID NO: 13, and the HCDR3 has a sequence of SEQ ID NO: 14.

In some embodiments, a polypeptide, an antibody, or antibody binding fragment thereof, comprises a heavy chain variable region having a HCDR1, a HCDR2, and a HCDR3, wherein the HCDR1 has a sequence of SEQ ID NO: 18, the HCDR2 has a sequence of SEQ ID NO: 19, and the HCDR3 has a sequence of SEQ ID NO: 14.

In some embodiments, a polypeptide, an antibody, or antibody binding fragment thereof, comprises a heavy chain variable region having a HCDR1, a HCDR2, and a HCDR3, wherein the HCDR1 has a sequence of SEQ ID NO: 20, the HCDR2 has a sequence of SEQ ID NO: 21, and the HCDR3 has a sequence of SEQ ID NO: 22.

In some embodiments, a polypeptide, an antibody or antibody binding fragment thereof, comprises: (i) a light chain having any one of the foregoing recited combinations of LCDR1, LCDR2, and LCDR3 sequences; and (ii) a heavy chain having any one of the foregoing recited combinations of HCDR1, HCDR2, and HCDR3 sequences.

The different CDR motifs can be combined in any combination including those not depicted in the table above. For example, the following embodiments are provided as non-limiting examples of such combinations.

In some embodiments, a polypeptide, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 15; the light chain CDR2 has the amino acid sequence of SEQ ID NO: 16; and the light chain CDR3 sequence has the amino acid sequence of SEQ ID NO: 17; and (ii) a heavy chain variable region comprising heavy chain CDR1, CDR2, and CDR3 sequences, wherein the heavy chain CDR1 sequence has the amino acid sequence of SEQ ID NO: 12; the heavy chain CDR2 sequence has the amino acid sequence of SEQ ID NO: 13; and the heavy chain CDR3 sequence has the amino acid sequence of SEQ ID NO: 14; or variants of any of the foregoing.

In some embodiments, a polypeptide, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 15; the light chain CDR2 has the amino acid sequence of SEQ ID NO: 16; and the light chain CDR3 sequence has the amino acid sequence of SEQ ID NO: 17; and (ii) a heavy chain variable region comprising heavy chain CDR1, CDR2, and CDR3 sequences, wherein the heavy chain CDR1 sequence has the amino acid sequence of SEQ ID NO: 18; the heavy chain CDR2 sequence has the amino acid sequence of SEQ ID NO: 19; and the heavy chain CDR3 sequence has the amino acid sequence of SEQ ID NO: 14; or variants of any of the foregoing.

In some embodiments, a polypeptide, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 23; the light chain CDR2 has the amino acid sequence of YA; and the light chain CDR3 sequence has the amino acid sequence of SEQ ID NO: 17; and (ii) a heavy chain variable region comprising heavy chain CDR1, CDR2, and CDR3 sequences, wherein the heavy chain CDR1 sequence has the amino acid sequence of SEQ ID NO: 20; the heavy chain CDR2 sequence has the amino acid sequence of SEQ ID NO: 21; and the heavy chain CDR3 sequence has the amino acid sequence of SEQ ID NO: 22; or variants of any of the foregoing.

In some embodiments, the light chain variable region CDR1 is replaced with any of the other light chain CDR1 sequences. In some embodiments, the light chain variable region CDR2 is replaced with any of the other light chain CDR2 sequences. In some embodiments, the light chain variable region CDR3 is replaced with any of the other light chain CDR3 sequences. In some embodiments, the heavy chain variable region CDR1 is replaced with any of the other heavy chain CDR1 sequences. In some embodiments, the heavy chain variable region CDR2 is replaced with any of the other heavy chain CDR2 sequences. In some embodiments, the heavy chain variable region CDR3 is replaced with any of the other heavy chain CDR3 sequences.

In some embodiments, the polypeptide comprises a heavy chain variable region peptide having one of the following sequences, or a variant thereof:

| SEQ ID NO: | AB ID NO. | Sequence |
|---|---|---|
| 24 | CD7AB1 | QVQLQQPGAELVKPGASVKLSC |
| | | KASGYPFTSYWIHWVKQRPGRG |
| | | LEWLGRIDPNSGDTKYNEKFKN |
| | | KATLTVDKSSTTAYMQLSSLTS |
| | | EDSAVYYCARSPYYSNDNSMDY |
| | | WGQGTSVTVSS |

In some embodiments, the polypeptide comprises a light chain variable region peptide having one of the following sequences, or a variant thereof:

| SEQ ID NO: | AB ID NO. | Sequence |
|---|---|---|
| 25 | CD7AB1 | DILLTQSPAILSVSPGERVSFSCR |
| | | ASQSIGTSIHWYQQRTNDSPRLLI |
| | | KYASESISGIPSRFSGSGSGTDFT |
| | | LSINSVESEDIADYYCQQSNSWPT |
| | | TFGGGTKLEIKR |

In some embodiments, a polypeptide, an antibody, or antigen binding fragment thereof, comprises a V_{H} peptide of SEQ ID NO: 24. In some embodiments, a polypeptide, an antibody, or antigen binding fragment thereof, comprises a V_{L} peptide of SEQ ID NO: 25. In some embodiments, a polypeptide, an antibody, or antigen binding fragment thereof, comprises a V_{H} peptide and a V_{L} peptide, wherein the wherein the V_{H} peptide comprises a sequence of SEQ ID NO: 24, or a variant thereof; and the V_{L} peptide comprises a sequence of SEQ ID NO: 25, or a variant thereof. In some embodiments, a polypeptide, an antibody, or antigen binding fragment thereof, comprises a V_{H} peptide and a V_{L} peptide, wherein the wherein the V_{H} peptide comprises a sequence of SEQ ID NO: 24, or a variant thereof; and the V_{L} peptide comprises a sequence of SEQ ID NO: 25, or a variant thereof, and the polypeptide, the antibody, or antigen binding fragment thereof, binds to non-human primate CD7. In some embodiments, a polypeptide, an antibody, or antigen binding fragment thereof, comprises a V_{H} peptide and a V_{L} peptide, wherein the wherein the V_{H} peptide comprises a sequence of SEQ ID NO: 24, or a variant thereof; and the V_{L} peptide comprises a sequence of SEQ ID NO: 25, or a variant thereof, and the polypeptide, the antibody, or antigen binding fragment thereof, binds to human CD7. In some embodiments, the V_{H} peptide comprises a sequence of SEQ ID NO: 24; and the V_{L} peptide comprises a sequence of SEQ ID NO: 25.

The VH and the VL sequences can be in any format, including, but not limited to an scFv format where the VH and VL regions are linked with a peptide linker. Examples of peptide linkers that can be used to link various peptides provided for herein include, but are not limited to: (GGGGS)ₙ (SEQ ID NO: 26), wherein each n is independently 1-5. In some embodiments, n is 1. In some embodiments, n is 2. In some embodiments, n is 3. In some embodiments, n is 4. In some embodiments, n is 5. In some embodiments, the variable regions are not linked with a peptide linker. In some embodiments, the polypeptide comprises SEQ ID NO: 24 and SEQ ID NO: 25.

In some embodiments, the VH and VL polypeptides are linked to a Fc region. In some embodiments, the Fc region is as provided for herein. In some embodiments, the Fc region comprises an amino acid sequence of SEQ ID NO: 9, SEQ ID NO: 10, or SEQ ID NO: 11 as provided for herein. As provided for herein, the heavy chain can be linked to a Fc region. Non-limiting mutations in the Fc region are provided for herein. In some embodiments, the Fc region further comprises a transmembrane domain. Examples of transmembrane domains include, but are not limited to, a CD8 or CD28 (“CD8/CD28”) transmembrane domain. In some embodiments, the Fc region further comprises a CD8 transmembrane domain. In some embodiments, the Fc region further comprises a CD28 transmembrane domain. In some embodiments, the Fc region comprising a transmembrane domain further comprises an Env incorporation motif. In some embodiments, the Fc region comprising a CD8/CD28 transmembrane domain further comprises an Env incorporation motif. In some embodiments, the VH and VL polypeptides provided herein are linked to an Fc region comprising a transmembrane domain. In some embodiments, the VH and VL polypeptides provided herein are linked to an Fc region comprising a CD8/CD28 transmembrane domain. In some embodiments, the VH and VL polypeptides provided herein are linked to an Fc region comprising a CD8/CD28 transmembrane domain and an Env incorporation motif. In some embodiments, the VH and VL polypeptides provided herein linked to an Fc region comprising a CD8/CD28 transmembrane domain are anchored to the plasma membrane on the surface of a cell. In some embodiments, the cell is an immune cell, such as those provided herein. In some embodiments, the VH having a sequence as set forth in SEQ ID NO: 24 and VL having a sequence as set forth in SEQ ID NO: 25 are linked to an Fc region comprising a transmembrane domain. In some embodiments, the VH having a sequence as set forth in SEQ ID NO: 24 and VL having a sequence as set forth in SEQ ID NO: 25 are linked to an Fc region comprising a CD8/CD28 transmembrane domain. In some embodiments, the VH having a sequence as set forth in SEQ ID NO: 24 and VL having a sequence as set forth in SEQ ID NO: 25 are linked to an Fc region comprising a CD8/CD28 transmembrane domain and an Env incorporation motif. In some embodiments, the VH having a sequence as set forth in SEQ ID NO: 24 and VL having a sequence as set forth in SEQ ID NO: 25 linked to an Fc region comprising a CD8/CD28 transmembrane domain are anchored to the plasma membrane on the surface of a cell. In some embodiments, the cell is an immune cell, such as those provided herein.

In some embodiments, the V_{H} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 25 are linked to an Fc region comprising a transmembrane domain. In some embodiments, the V_{H} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 25 are linked to an Fc region comprising a CD8/CD28 transmembrane domain. In some embodiments, the V_{H} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 25 are linked to an Fc region comprising a CD8/CD28 transmembrane domain and an Env incorporation motif. In some embodiments, the V_{H} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 25 linked to an Fc region comprising a CD8/CD28 transmembrane domain are anchored to the plasma membrane on the surface of a cell. In some embodiments, the cell is an immune cell, such as those provided herein.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 25.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 25; provided that the V_{H} peptide and a V_{L} peptide comprises a light chain CDR having a sequence of SEQ ID NO: 15-17; and/or a heavy chain CDR having a sequence of SEQ ID NO: 12-14. In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 25; provided that the V_{H} peptide and a V_{L} peptide comprise a light chain CDR1 having a sequence of SEQ ID NO: 15; a light chain CDR2 having a sequence of SEQ ID NO: 16; a light chain CDR3 having a sequence of SEQ ID NO: 17; and/or a heavy chain CDR1 having a sequence of SEQ ID NO: 12; a heavy chain CDR2 having a sequence of SEQ ID NO: 13; and a heavy chain CDR3 having a sequence of SEQ ID NO: 14. In some embodiments, the CDRs in the V_{H} or V_{L} chain are as set forth in the combinations provided for herein.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 25; provided that the V_{L} peptide comprises a LCDR1 having a sequence of SEQ ID NO: 15; a LCDR2 having a sequence of SEQ ID NO: 16; and a LCDR3 having a sequence of SEQ ID NO: 17; and the V_{H} peptide comprises a HCDR1 having a sequence of SEQ ID NO: 12; a HCDR2 having a sequence of SEQ ID NO: 13; and a HCDR3 having a sequence of SEQ ID NO: 14.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 25; provided that the V_{L} peptide comprises a LCDR1 having a sequence of SEQ ID NO: 15, wherein the LCDR1 comprises at most 1 conservative amino acid substitution, a LCDR2 having a sequence of SEQ ID NO: 16, wherein the LCDR2 comprises at most 1 conservative amino acid substitution, and a LCDR3 having a sequence of SEQ ID NO: 17, wherein the LCDR3 comprises at most 1 conservative amino acid substitution; and the V_{H} peptide comprises a HCDR1 having a sequence of SEQ ID NO: 12, wherein the HCDR1 comprises at most 1 conservative amino acid substitution, a HCDR2 having a sequence of SEQ ID NO: 13, wherein the HCDR2 comprises at most 1 conservative amino acid substitution, and a HCDR3 having a sequence of SEQ ID NO: 14, wherein the HCDR3 comprises at most 1 conservative amino acid substitution.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 25; provided that the V_{H} peptide and a V_{L} peptide comprise a light chain CDR1 having a sequence of SEQ ID NO: 15; a light chain CDR2 having a sequence of SEQ ID NO: 16; a light chain CDR1 having a sequence of SEQ ID NO: 17; and/or a heavy chain CDR1 having a sequence of SEQ ID NO: 18; a heavy chain CDR2 having a sequence of SEQ ID NO: 19; and a heavy chain CDR3 having a sequence of SEQ ID NO: 14. In some embodiments, the CDRs in the V_{H} or V_{L} chain are as set forth in the combinations provided for herein.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 25; provided that the V_{L} peptide comprises a LCDR1 having a sequence of SEQ ID NO: 15; a LCDR2 having a sequence of SEQ ID NO: 16; and a LCDR3 having a sequence of SEQ ID NO: 17; and the V_{H} peptide comprises a HCDR1 having a sequence of SEQ ID NO: 18; a HCDR2 having a sequence of SEQ ID NO: 19; and a HCDR3 having a sequence of SEQ ID NO: 14.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 25; provided that the V_{L} peptide comprises a LCDR1 having a sequence of SEQ ID NO: 15, wherein the LCDR1 comprises at most 1 conservative amino acid substitution, a LCDR2 having a sequence of SEQ ID NO: 16, wherein the LCDR2 comprises at most 1 conservative amino acid substitution, and a LCDR3 having a sequence of SEQ ID NO: 17, wherein the LCDR3 comprises at most 1 conservative amino acid substitution; and the V_{H} peptide comprises a HCDR1 having a sequence of SEQ ID NO: 18, wherein the HCDR1 comprises at most 1 conservative amino acid substitution, a HCDR2 having a sequence of SEQ ID NO: 19, wherein the HCDR2 comprises at most 1 conservative amino acid substitution, and a HCDR3 having a sequence of SEQ ID NO: 14, wherein the HCDR3 comprises at most 1 conservative amino acid substitution.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 25; provided that the V_{H} peptide and a V_{L} peptide comprise a light chain CDR1 having a sequence of SEQ ID NO: 23; a light chain CDR2 having a sequence of YA; a light chain CDR1 having a sequence of SEQ ID NO: 17; and/or a heavy chain CDR1 having a sequence of SEQ ID NO: 20; a heavy chain CDR2 having a sequence of SEQ ID NO: 21; and a heavy chain CDR3 having a sequence of SEQ ID NO: 22. In some embodiments, the CDRs in the V_{H} or V_{L} chain are as set forth in the combinations provided for herein.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 25; provided that the V_{L} peptide comprises a LCDR1 having a sequence of SEQ ID NO: 23; a LCDR2 having a sequence of YA; and a LCDR3 having a sequence of SEQ ID NO: 17; and the V_{H} peptide comprises a HCDR1 having a sequence of SEQ ID NO: 20; a HCDR2 having a sequence of SEQ ID NO: 21; and a HCDR3 having a sequence of SEQ ID NO: 22.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 24; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 25; provided that the V_{L} peptide comprises a LCDR1 having a sequence of SEQ ID NO: 23, wherein the LCDR1 comprises at most 1 conservative amino acid substitution, a LCDR2 having a sequence of YA, wherein the LCDR2 comprises at most 1 conservative amino acid substitution, and a LCDR3 having a sequence of SEQ ID NO: 17, wherein the LCDR3 comprises at most 1 conservative amino acid substitution; and the V_{H} peptide comprises a HCDR1 having a sequence of SEQ ID NO: 20, wherein the HCDR1 comprises at most 1 conservative amino acid substitution, a HCDR2 having a sequence of SEQ ID NO: 21, wherein the HCDR2 comprises at most 1 conservative amino acid substitution, and a HCDR3 having a sequence of SEQ ID NO: 22, wherein the HCDR3 comprises at most 1 conservative amino acid substitution.

In some embodiments, a polypeptide comprises a V_{H} peptide and a V_{L} peptide, wherein the V_{H} peptide comprises a sequence of SEQ ID NO: 24 and the V_{L} peptide comprises a sequence of SEQ ID NO: 25.

In some embodiments, a polypeptide as provided herein binds to non-human primate CD7. In some embodiments, a polypeptide as provided herein binds to human CD7.

As provided for herein, the different polypeptides (V_{H} or V_{L}) described herein can be linked with a peptide linker or not linked with a peptide linker and instead for a continuous sequence. In some embodiments, the peptide linker comprises a sequence of (GGGGS)ₙ (SEQ ID NO: 26), wherein each n is independently 1-5. In some embodiments, n is 1. In some embodiments, n is 2. In some embodiments, n is 3. In some embodiments, n is 4. In some embodiments, n is 5. The linked peptide format can be represented by a formula of V_{H}-Z-V_{L} or V_{L}-Z-V_{H}, wherein Z is the peptide linker. In some embodiments, Z is (GGGGS)ₙ (SEQ ID NO: 26), wherein each n is independently 1-5.

In some embodiments, a polypeptide comprising the linked peptide represented by a formula of V_{L}-Z-V_{H} comprises a heavy chain variable region as set forth in SEQ ID NO: 24 linked via a linker sequence GGGGSGGGGSGGGGSGGGGS (SEQ ID NO: 27) to a light chain variable region as set forth in SEQ ID NO: 25. In some embodiments, a polypeptide comprising a V_{L} linked via a peptide linker to a V_{H} has the sequence as set forth below,

(SEQ ID NO: 28)
DILLTQSPAILSVSPGERVSFSCRASQSIGTSIHW
YQQRTNDSPRLLIKYASESISGIPSRFSGSGSGTD
FTLSINSVESEDIADYYCQQSNSWPTTFGGGTKLE
IKRGGGGSGGGGSGGGGSGGGGSQVQLQQPGAELV
KPGASVKLSCKASGYPFTSYWIHWVKQRPGRGLEW
LGRIDPNSGDTKYNEKFKNKATLTVDKSSTTAYMQ
LSSLTSEDSAVYYCARSPYYSNDNSMDYWGQGTSV
TVSS

In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 28. In some embodiments, a polypeptide comprises a sequence that is at least 90% identical to a sequence of SEQ ID NO: 28. In some embodiments, a polypeptide comprises a sequence that is at least 95% identical to a sequence of SEQ ID NO: 28. In some embodiments, a polypeptide comprises a sequence that is at least 99% identical to a sequence of SEQ ID NO: 28. In some embodiments, a polypeptide comprises a sequence as set forth in SEQ ID NO: 28. In some embodiments, the polypeptide as set forth in SEQ ID NO: 28 is an antibody, or an antigen binding fragment thereof. In some embodiments, the antibody is an anti-CD7 antibody.

In some embodiments, a polypeptide comprising the linked peptide represented by a formula of V_{H}-Z-V_{L} comprises a light chain variable region as set forth in SEQ ID NO: 25 linked via a linker sequence GGGGSGGGGSGGGGSGGGGS (SEQ ID NO: 27) to a heavy chain variable region as set forth in SEQ ID NO: 24. In some embodiments, a polypeptide comprising a V_{H} linked via a peptide linker to a V_{L} has the sequence as set forth below:

(SEQ ID NO: 29)
QVQLQQPGAELVKPGASVKLSCKASGYPFTSYWIH
WVKQRPGRGLEWLGRIDPNSGDTKYNEKFKNKATL
TVDKSSTTAYMQLSSLTSEDSAVYYCARSPYYSND
NSMDYWGQGTSVTVSSGGGGSGGGGSGGGGSGGGG
SDILLTQSPAILSVSPGERVSFSCRASQSIGTSIH
WYQQRTNDSPRLLIKYASESISGIPSRFSGSGSGT
DFTLSINSVESEDIADYYCQQSNSWPTTFGGGTKL
EIKR

In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 29. In some embodiments, a polypeptide comprises a sequence that is at least 90% identical to a sequence of SEQ ID NO: 29. In some embodiments, a polypeptide comprises a sequence that is at least 95% identical to a sequence of SEQ ID NO: 29. In some embodiments, a polypeptide comprises a sequence that is at least 99% identical to a sequence of SEQ ID NO: 29. In some embodiments, a polypeptide comprises a sequence as set forth in SEQ ID NO: 29. In some embodiments, the polypeptide as set forth in SEQ ID NO: 29 is an antibody, or an antigen binding fragment thereof. In some embodiments, the antibody is an anti-CD7 antibody. In some embodiments, the anti-CD7 antibody binds to non-human primate CD7. In some embodiments, the anti-CD7 antibody binds to human CD7.

In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 28 and comprises an Fc region, such as those provided herein. In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 28 and comprises an Fc region, such as those provided herein, and a transmembrane domain, such as those provided herein. In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 28 and comprises an Fc region, such as those provided herein, and a CD8/CD28 transmembrane domain. In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 28 and comprises an Fc region, such as those provided herein, a transmembrane domain, such as those provided herein, and an Env incorporation motif.

In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 29 and comprises an Fc region, such as those provided herein. In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 29 and comprises an Fc region, such as those provided herein, and a transmembrane domain, such as those provided herein. In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 29 and comprises an Fc region, such as those provided herein, and a CD8/CD28 transmembrane domain. In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 29 and comprises an Fc region, such as those provided herein, a transmembrane domain, such as those provided herein, and an Env incorporation motif.

As provided for herein, the polypeptide, antibodies, or antigen binding fragments thereof can be variants of the sequences.

The sequences of the polypeptides or antibodies can be modified to yield human IgG antibodies. The conversion of the sequences provided herein can be modified to yield other types of antibodies. The CDRs can also be linked to other antibodies, proteins, or molecules to create antibody fragments that bind CD7.

In some embodiments, a polypeptide or an antibody as provided herein is a targeting moiety on the surface of an engineered viral particle. In some embodiments, the targeting moiety allows for binding to a target cell. In some embodiments, the targeting moiety is a CD7 binding moiety, such as a polypeptide or an antibody as provided herein. In some embodiments, the targeting moiety comprises a sequence that is at least 85%, 86%, 87%, 88%, 89%, 90%, 91%, 92%, 93%, 94%, 95%, 96%, 97%, 98%, or 99% identical to a sequence of SEQ ID NO: 28. In some embodiments, the targeting moiety comprises a sequence that is at least 90% identical to a sequence of SEQ ID NO: 28. In some embodiments, the targeting moiety comprises a sequence that is at least 95% identical to a sequence of SEQ ID NO: 28. In some embodiments, the targeting moiety comprises a sequence that is at least 99% identical to a sequence of SEQ ID NO: 28. In some embodiments, the targeting moiety comprises a sequence as set forth in SEQ ID NO: 28. In some embodiments, the targeting moiety as set forth in SEQ ID NO: 28 is an antibody, or an antigen binding fragment thereof. In some embodiments, the targeting moiety is an anti-CD7 antibody.

In some embodiments, a polypeptide or an antibody as provided for herein is a targeting moiety on the surface of an engineered viral particle. In some embodiments, the engineered viral particle is a pseudotyped viral-like particle. In some embodiments, the targeting moiety allows for binding to a target cell. In some embodiments, the targeting moiety is a CD7 binding moiety, such as a polypeptide or an antibody as provided herein. In some embodiments, the targeting moiety comprises a sequence that is at least 85%, 86%, 87%, 88%, 89%, 90%, 91%, 92%, 93%, 94%, 95%, 96%, 97%, 98%, or 99% identical to a sequence of SEQ ID NO: 29. In some embodiments, the targeting moiety comprises a sequence that is at least 90% identical to a sequence of SEQ ID NO: 29. In some embodiments, the targeting moiety comprises a sequence that is at least 95% identical to a sequence of SEQ ID NO: 29. In some embodiments, the targeting moiety comprises a sequence that is at least 99% identical to a sequence of SEQ ID NO: 29. In some embodiments, the targeting moiety comprises a sequence as set forth in SEQ ID NO: 29. In some embodiments, the targeting moiety as set forth in SEQ ID NO: 29 is an antibody, or an antigen binding fragment thereof. In some embodiments, the targeting moiety is an anti-CD7 antibody. In some embodiments, the anti-CD7 antibody binds to non-human primate CD7. In some embodiments, the anti-CD7 antibody binds to human CD7.

In some embodiments, a polypeptide comprises a sequence having a sequence as set forth in SEQ ID NO: 28 and comprises an Fc region, such as those provided herein. In some embodiments, a polypeptide comprises a sequence having a sequence as set forth in SEQ ID NO: 28 and comprises an Fc region, such as those provided herein, and a transmembrane domain, such as those provided herein. In some embodiments, a polypeptide comprises a sequence having a sequence as set forth in SEQ ID NO: 28 and comprises an Fc region, such as those provided herein, and a CD8/CD28 transmembrane domain. In some embodiments, a polypeptide comprises a sequence having a sequence as set forth in SEQ ID NO: 28 and comprises an Fc region, such as those provided herein, a transmembrane domain, such as those provided herein, and an Env incorporation motif.

In some embodiments, a polypeptide comprises a sequence having a sequence as set forth in SEQ ID NO: 29 and comprises an Fc region, such as those provided herein. In some embodiments, a polypeptide comprises a sequence having a sequence as set forth in SEQ ID NO: 29 and comprises an Fc region, such as those provided herein, and a transmembrane domain, such as those provided herein. In some embodiments, a polypeptide comprises a sequence having a sequence as set forth in SEQ ID NO: 29 and comprises an Fc region, such as those provided herein, and a CD8/CD28 transmembrane domain. In some embodiments, a polypeptide comprises a sequence having a sequence as set forth in SEQ ID NO: 29 and comprises an Fc region, such as those provided herein, a transmembrane domain, such as those provided herein, and an Env incorporation motif.

In some embodiments, the targeting moiety (a polypeptide) can bind to CD8.

In some embodiments, the polypeptide binds to CD8. In some embodiments, the polypeptide binds to CD8-alpha. In some embodiments, the polypeptide binds to CD8-beta. In some embodiments, the polypeptide binds to CD8 heterodimer. In some embodiments, the CD8 heterodimer comprises CD8-alpha and CD8-beta subunits. In some embodiments, the polypeptide binds to CD8-alpha homodimer. In some embodiments, the polypeptide that binds to CD8 is an antibody which binds to non-human primate CD8. In some embodiments, the antibody that binds to non-human primate CD8 is an antibody which binds to non-human primate CD8-alpha. In some embodiments, the antibody that binds to non-human primate CD8 is an antibody which binds to non-human primate CD8-beta. In some embodiments, the antibody that binds to non-human primate CD8 is an antibody which binds to non-human primate CD8-alpha homodimer. In some embodiments, the antibody that binds to non-human primate CD8 is an antibody which binds to non-human primate CD8 heterodimer. In some embodiments, the polypeptide that binds to CD8 is an antibody which binds to human CD8. In some embodiments, the antibody that binds to human CD8 is an antibody which binds to human CD8-alpha. In some embodiments, the antibody that binds to human CD8 is an antibody which binds to human CD8-beta. In some embodiments, the antibody that binds to human CD8 is an antibody which binds to human CD8-alpha homodimer. In some embodiments, the antibody that binds to human CD8 is an antibody which binds to human CD8 heterodimer. The sequence of human CD8-alpha (UniProtKB Q8TAW8) is as follows (SEQ ID NO: 30):

(SEQ ID NO: 30)
MALPVTALLLPLALLLHAARPSQFRVSPLDRTWNL
GETVELKCQVLLSNPTSGCSWLFQPRGAAASPTFL
LYLSQNKPKAAEGLDTQRFSGKRLGDTFVLTLSDF
RRENEGCYFCSALSNSIMYFSHFVPVFLPAKPTTT
PAPRPPTPAPTIASQPLSLRPEACRPAAGGAVHTR
GLDFACDIYIWAPLAGTCGVLLLSLVITLYCNHRN
RRRVCKCPRPVVKSGDKPSLSARYV

The sequence of human CD8-beta (UniProtKB Q8TD28) is as follows (SEQ ID NO: 31):

(SEQ ID NO: 31)
MRPRLWLLLAAQLTVLHGNSVLQQTPAYIKVQTNK
MVMLSCEAKISLSNMRIYWLRQRQAPSSDSHHEFL
ALWDSAKGTIHGEEVEQEKIAVFRDASRFILNLTS
VKPEDSGIYFCMIVGSPELTFGKGTQLSVVDFLPT
TAQPTKKSTLKKRVCRLPRPETQKGPLCSPITLGL
LVAGVLVLLVSLGVAIHLCCRRRRARLRFMKQLYK

In some embodiments, the CD8 that the polypeptide binds to is expressed on the surface of a cell. In some embodiments, the cell is an immune cell. In some embodiments, the immune cell is a CD7+ T cell, CD4+ T cell, CD8+ T cell, NK cell, alpha-beta T cell, gamma-delta T cell, lymphoid progenitor cell, hematopoietic stem cell, myeloid cell, monocyte, macrophage, central memory T cell, effector memory T cell, stem-cell like memory T cells, naïve T cell, activated T cell, regulatory T cell (TReg), terminally differentiated effector memory T cell (TEMRA), resident memory T cell (TRM) or a T-cell CD8+ CCR7+. In some embodiments, the cell is a CD8+ T cell. In some embodiments, the cell is a CD8+ cell.

In some embodiments, the antibody comprises a Fc region. The Fc region can be linked to the heavy or light chain of the antibody. In some embodiments, the Fc region is an IgG Fc. In some embodiments, the IgG is selected from IgG1, IgG2, IgG3, or IgG4. In some embodiments, the IgG Fc is IgG1 Fc and comprises an amino acid sequence of SEQ ID NO: 9 or a variant thereof as provided for herein. In some embodiments, the IgG Fc is IgG2 Fc and comprises an amino acid sequence of SEQ ID NO: 10 or a variant thereof as provided for herein. In some embodiments, the IgG Fc is IgG4 Fc and comprises an amino acid sequence of SEQ ID NO: 11 or a variant thereof as provided for herein.

In some embodiments, polypeptides (e.g. CD8-binding polypeptide) are provided herein. In some embodiments, antibodies (e.g. an anti-CD8 antibody) are provided herein. In some embodiments, the antibody is a recombinant antibody that binds to CD8. In some embodiments, the CD8 protein is a human CD8 protein. In some embodiments, the CD8 protein is a non-human CD8 protein (e.g., mouse, rat, pig, dog, non-human primate). As used herein, the term “recombinant antibody” refers to an antibody that is not naturally occurring. In some embodiments, the term “recombinant antibody” refers to an antibody that is not isolated from a human subject.

In some embodiments, an antibody, or antigen binding fragment thereof is provided, wherein the antibody or antibody fragment comprises a peptide selected from the following table, which illustrate the CDRs based on Chothia numbering.

Chothia CDRS

| Ab ID | | | | | | |
|---|---|---|---|---|---|---|
| No | HCDR1 | HCDR2 | HCDR3 | LCDR1 | LCDR2 | LCDR3 |
| CD8AB1 | RYTF | YPYN | DHRYN | RASE | LASN | QQNNE |
| | TDY | GG | EGVSF | SVDG | LES | DPYT |
| | (SEQ | (SEQ | DY | FGNS | (SEQ | (SEQ |
| | ID | ID | (SEQ | FMN | ID | ID |
| | NO: | NO: | ID | (SEQ | NO: | NO: |
| | 32) | 33) | NO: | ID | 36) | 37) |
| | | | 34) | NO: | | |
| | | | | 35) | | |

In some embodiments, an antibody, or antigen binding fragment thereof is provided, wherein the antibody or antibody fragment comprises a peptide selected from the following table, which illustrate the CDRs based on Kabat numbering.

Kabat CDRS

| Ab ID No | HCDR1 | HCDR2 | HCDR3 | LCDR1 | LCDR2 | LCDR3 |
|---|---|---|---|---|---|---|
| CD8AB1 | DYNLH | FIYPY | DHRYN | RASE | LASN | QQNNE |
| | (SEQ | NGGT | EGVSF | SVDG | LES | DPYT |
| | ID | GYNQ | DY | FGNS | (SEQ | (SEQ |
| | NO: | KFKN | (SEQ | FMN | ID | ID |
| | 38) | (SEQ | ID | (SEQ | NO: | NO: |
| | | ID | NO: | ID | 36) | 37) |
| | | NO: | 34) | NO: | | |
| | | 39) | | 35) | | |

In some embodiments, an antibody, or antigen binding fragment thereof is provided, wherein the antibody or antibody fragment comprises a peptide selected from the following table, which illustrate the CDRs based on IMGT numbering.

IMGT CDRS

| Ab ID | | | | | | |
|---|---|---|---|---|---|---|
| No | HCDR1 | HCDR2 | HCDR3 | LCDR1 | LCDR2 | LCDR3 |
| CD8AB1 | RYTF | TYPY | ARDHR | ESVD | LA | QQNNE |
| | TDYN | NGGT | YNEGV | GFGN | | DPYT |
| | (SEQ | (SEQ | SFDY | SF | | (SEQ |
| | ID | ID | (SEQ | (SEQ | | ID |
| | NO: | NO: | ID | ID | | NO: |
| | 40) | 41) | NO: | NO: | | 37) |
| | | | 42) | 43) | | |

In some embodiments, a polypeptide, an antibody, or antibody binding fragment thereof, comprises a heavy or light chain CDR as provided in the tables above. In some embodiments, a polypeptide, an antibody, or antibody binding fragment thereof, comprises a heavy or light chain CDR as provided in the tables above and binds to non-human primate CD8. In some embodiments, a polypeptide, an antibody, or antibody binding fragment thereof, comprises a heavy or light chain CDR as provided in the tables above and binds to human CD8. In some embodiments, a polypeptide, an antibody, or antibody binding fragment thereof, comprises a light chain CDR having a sequence selected from SEQ ID NO: 35-37. In some embodiments, a polypeptide, an antibody, or antibody binding fragment thereof, comprises a light chain CDR having a sequence of SEQ ID NO: 35. In some embodiments, a polypeptide, an antibody, or antibody binding fragment thereof, comprises a light chain CDR having a sequence of SEQ ID NO: 36. In some embodiments, a polypeptide, an antibody, or antibody binding fragment thereof, comprises a light chain CDR having a sequence of SEQ ID NO: 37. In some embodiments, a polypeptide, an antibody, or antibody binding fragment thereof, comprises a heavy chain CDR having a sequence selected from SEQ ID NO: 32-34. In some embodiments, a polypeptide, an antibody, or antibody binding fragment thereof, comprises a heavy chain CDR having a sequence of SEQ ID NO: 32. In some embodiments, a polypeptide, an antibody, or antibody binding fragment thereof, comprises a heavy chain CDR having a sequence of SEQ ID NO: 33. In some embodiments, a polypeptide, an antibody, or antibody binding fragment thereof, comprises a heavy chain CDR having a sequence of SEQ ID NO: 34. The CDRs referenced in the embodiments throughout the present specification can be interchanged with the CDRs that are characterized by different formats, such as Kabat and IMGT.

In some embodiments, a polypeptide, an antibody, or antibody binding fragment thereof, comprises a light chain variable region having a LCDR1, a LCDR2, and a LCDR3, wherein the LCDR1 has a sequence of SEQ ID NO: 35, the LCDR2 has a sequence of SEQ ID NO: 36, and the LCDR3 has a sequence of SEQ ID NO: 37.

In some embodiments, a polypeptide, an antibody, or antibody binding fragment thereof, comprises a light chain variable region having a LCDR1, a LCDR2, and a LCDR3, wherein the LCDR1 has a sequence of SEQ ID NO: 43, the LCDR2 has a sequence of LA, and the LCDR3 has a sequence of SEQ ID NO: 37.

In some embodiments, a polypeptide, an antibody, or antibody binding fragment thereof, comprises a heavy chain variable region having a HCDR1, a HCDR2, and a HCDR3, wherein the HCDR1 has a sequence of SEQ ID NO: 32, the HCDR2 has a sequence of SEQ ID NO: 33, and the HCDR3 has a sequence of SEQ ID NO: 34.

In some embodiments, a polypeptide, an antibody, or antibody binding fragment thereof, comprises a heavy chain variable region having a HCDR1, a HCDR2, and a HCDR3, wherein the HCDR1 has a sequence of SEQ ID NO: 38, the HCDR2 has a sequence of SEQ ID NO: 39, and the HCDR3 has a sequence of SEQ ID NO: 34.

In some embodiments, a polypeptide, an antibody, or antibody binding fragment thereof, comprises a heavy chain variable region having a HCDR1, a HCDR2, and a HCDR3, wherein the HCDR1 has a sequence of SEQ ID NO: 40, the HCDR2 has a sequence of SEQ ID NO: 41, and the HCDR3 has a sequence of SEQ ID NO: 42.

In some embodiments, a polypeptide, an antibody or antibody binding fragment thereof, comprises: (i) a light chain having any one of the foregoing recited combinations of LCDR1, LCDR2, and LCDR3 sequences; and (ii) a heavy chain having any one of the foregoing recited combinations of HCDR1, HCDR2, and HCDR3 sequences.

The different CDR motifs can be combined in any combination including those not depicted in the table above. For example, the following embodiments are provided as non-limiting examples of such combinations.

In some embodiments, a polypeptide, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 35; the light chain CDR2 has the amino acid sequence of SEQ ID NO: 36; and the light chain CDR3 sequence has the amino acid sequence of SEQ ID NO: 37; and (ii) a heavy chain variable region comprising heavy chain CDR1, CDR2, and CDR3 sequences, wherein the heavy chain CDR1 sequence has the amino acid sequence of SEQ ID NO: 32; the heavy chain CDR2 sequence has the amino acid sequence of SEQ ID NO: 33; and the heavy chain CDR3 sequence has the amino acid sequence of SEQ ID NO: 34; or variants of any of the foregoing.

In some embodiments, a polypeptide, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 35; the light chain CDR2 has the amino acid sequence of SEQ ID NO: 36; and the light chain CDR3 sequence has the amino acid sequence of SEQ ID NO: 37; and (ii) a heavy chain variable region comprising heavy chain CDR1, CDR2, and CDR3 sequences, wherein the heavy chain CDR1 sequence has the amino acid sequence of SEQ ID NO: 38; the heavy chain CDR2 sequence has the amino acid sequence of SEQ ID NO: 39; and the heavy chain CDR3 sequence has the amino acid sequence of SEQ ID NO: 34; or variants of any of the foregoing.

In some embodiments, a polypeptide, an antibody, or antigen binding fragment thereof, comprises: (i) a light chain variable region comprising light chain CDR1, CDR2, and CDR3 sequences, wherein the light chain CDR1 sequence has the amino acid sequence of SEQ ID NO: 43; the light chain CDR2 has the amino acid sequence of LA; and the light chain CDR3 sequence has the amino acid sequence of SEQ ID NO: 37; and (ii) a heavy chain variable region comprising heavy chain CDR1, CDR2, and CDR3 sequences, wherein the heavy chain CDR1 sequence has the amino acid sequence of SEQ ID NO: 40; the heavy chain CDR2 sequence has the amino acid sequence of SEQ ID NO: 41; and the heavy chain CDR3 sequence has the amino acid sequence of SEQ ID NO: 42; or variants of any of the foregoing.

In some embodiments, the light chain variable region CDR1 is replaced with any of the other light chain CDR1 sequences. In some embodiments, the light chain variable region CDR2 is replaced with any of the other light chain CDR2 sequences. In some embodiments, the light chain variable region CDR3 is replaced with any of the other light chain CDR3 sequences. In some embodiments, the heavy chain variable region CDR1 is replaced with any of the other heavy chain CDR1 sequences. In some embodiments, the heavy chain variable region CDR2 is replaced with any of the other heavy chain CDR2 sequences. In some embodiments, the heavy chain variable region CDR3 is replaced with any of the other heavy chain CDR3 sequences.

In some embodiments, the polypeptide comprises a heavy chain variable region peptide having one of the following sequences, or a variant thereof:

| SEQ ID NO: | AB ID NO. | Sequence |
|---|---|---|
| 44 | CD8AB1 | EVQLQQSGPELVKPGASVKISCK |
| | | ASRYTFTDYNLHWVKLSHEKSLE |
| | | WIGFIYPYNGGTGYNQKFKNKAK |
| | | LTVDYSSSTAYMELRSLTSVDAA |
| | | VYYCARDHRYNEGVSFDYWGQGT |
| | | TLTVSS |

In some embodiments, the polypeptide comprises a light chain variable region peptide having one of the following sequences, or a variant thereof:

| SEQ ID NO: | AB ID NO. | Sequence |
|---|---|---|
| 45 | CD8AB1 | NIVLTQSPASLAVSLGQRATISCRA |
| | | SESVDGFGNSFMNWYQQKPGQSPKL |
| | | LIYLASNLESGVPARFSGSGSRTDF |
| | | TLTIDPVEADDAATYYCQQNNEDPY |
| | | TFGGGTKLEIKR |

In some embodiments, a polypeptide, an antibody, or antigen binding fragment thereof, comprises a V_{H} peptide of SEQ ID NO: 44. In some embodiments, a polypeptide, an antibody, or antigen binding fragment thereof, comprises a V_{L} peptide of SEQ ID NO: 45. In some embodiments, a polypeptide, an antibody, or antigen binding fragment thereof, comprises a V_{H} peptide and a V_{L} peptide, wherein the wherein the V_{H} peptide comprises a sequence of SEQ ID NO: 44, or a variant thereof; and the V_{L} peptide comprises a sequence of SEQ ID NO: 45, or a variant thereof. In some embodiments, a polypeptide, an antibody, or antigen binding fragment thereof, comprises a V_{H} peptide and a V_{L} peptide, wherein the wherein the V_{H} peptide comprises a sequence of SEQ ID NO: 44, or a variant thereof; and the V_{L} peptide comprises a sequence of SEQ ID NO: 45, or a variant thereof, and the polypeptide, the antibody, or antigen binding fragment thereof, binds to non-human primate CD8. In some embodiments, a polypeptide, an antibody, or antigen binding fragment thereof, comprises a V_{H} peptide and a V_{L} peptide, wherein the wherein the V_{H} peptide comprises a sequence of SEQ ID NO: 44, or a variant thereof; and the V_{L} peptide comprises a sequence of SEQ ID NO: 45, or a variant thereof, and the polypeptide, the antibody, or antigen binding fragment thereof, binds to human CD8. In some embodiments, the V_{H} peptide comprises a sequence of SEQ ID NO: 44; and the V_{L} peptide comprises a sequence of SEQ ID NO: 45.

The VH and the VL sequences can be in any format, including, but not limited to an scFv format where the VH and VL regions are linked with a peptide linker. Examples of peptide linkers that can be used to link various peptides provided for herein include, but are not limited to: (GGGGS)ₙ (SEQ ID NO: 26), wherein each n is independently 1-5. In some embodiments, n is 1. In some embodiments, n is 2. In some embodiments, n is 3. In some embodiments, n is 4. In some embodiments, n is 5. In some embodiments, the variable regions are not linked with a peptide linker. In some embodiments, the polypeptide comprises SEQ ID NO: 44 and SEQ ID NO: 45.

In some embodiments, the VH and VL polypeptides are linked to a Fc region. In some embodiments, the Fc region is as provided for herein. As provided for herein, the heavy chain can be linked to a Fc region. Non-limiting mutations in the Fc region are provided for herein. In some embodiments, the Fc region further comprises a transmembrane domain. Examples of transmembrane domains include, but are not limited to, a CD8/CD28 transmembrane domain. In some embodiments, the Fc region further comprises a CD8 or CD28 (“CD8/CD28”) transmembrane domain. In some embodiments, the Fc region further comprises a CD8 transmembrane domain. In some embodiments, the Fc region further comprises a CD28 transmembrane domain. In some embodiments, the Fc region comprising a transmembrane domain further comprises an Env incorporation motif. In some embodiments, the Fc region comprising a CD8/CD28 transmembrane domain further comprises an Env incorporation motif. In some embodiments, the VH and VL polypeptides provided herein are linked to an Fc region comprising a transmembrane domain. In some embodiments, the VH and VL polypeptides provided herein are linked to an Fc region comprising a CD8/CD28 transmembrane domain. In some embodiments, the VH and VL polypeptides provided herein are linked to an Fc region comprising a CD8/CD28 transmembrane domain and an Env incorporation motif. In some embodiments, the VH and VL polypeptides provided herein linked to an Fc region comprising a CD8/CD28 transmembrane domain are anchored to the plasma membrane on the surface of a cell. In some embodiments, the cell is an immune cell, such as those provided herein. In some embodiments, the VH having a sequence as set forth in SEQ ID NO: 44 and VL having a sequence as set forth in SEQ ID NO: 45 are linked to an Fc region comprising a transmembrane domain. In some embodiments, the VH having a sequence as set forth in SEQ ID NO: 44 and VL having a sequence as set forth in SEQ ID NO: 45 are linked to an Fc region comprising a CD8/CD28 transmembrane domain. In some embodiments, the VH having a sequence as set forth in SEQ ID NO: 44 and VL having a sequence as set forth in SEQ ID NO: 45 are linked to an Fc region comprising a CD8/CD28 transmembrane domain and an Env incorporation motif. In some embodiments, the VH having a sequence as set forth in SEQ ID NO: 44 and VL having a sequence as set forth in SEQ ID NO: 45 linked to an Fc region comprising a CD8/CD28 transmembrane domain are anchored to the plasma membrane on the surface of a cell. In some embodiments, the cell is an immune cell, such as those provided herein.

In some embodiments, the V_{H} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 45 are linked to an Fc region comprising a transmembrane domain. In some embodiments, the V_{H} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 45 are linked to an Fc region comprising a CD8/CD28 transmembrane domain. In some embodiments, the V_{H} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 45 are linked to an Fc region comprising a CD8/CD28 transmembrane domain and an Env incorporation motif. In some embodiments, the V_{H} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprising a sequence that is at least 85%, 86%, 87%, 88%, 89%, 90%, 91%, 92%, 93%, 94%, 95%, 96%, 97%, 98%, or 99% identical to a sequence of SEQ ID NO: 45 linked to an Fc region comprising a CD8/CD28 transmembrane domain are anchored to the plasma membrane on the surface of a cell. In some embodiments, the cell is an immune cell, such as those provided herein.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 45.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 45; provided that the V_{H} peptide and a V_{L} peptide comprises a light chain CDR having a sequence of SEQ ID NO: 35-37; and/or a heavy chain CDR having a sequence of SEQ ID NO: 32-34. In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 45; provided that the V_{H} peptide and a V_{L} peptide comprise a light chain CDR1 having a sequence of SEQ ID NO: 35; a light chain CDR2 having a sequence of SEQ ID NO: 36; a light chain CDR3 having a sequence of SEQ ID NO: 37; and/or a heavy chain CDR1 having a sequence of SEQ ID NO: 32; a heavy chain CDR2 having a sequence of SEQ ID NO: 33; and a heavy chain CDR3 having a sequence of SEQ ID NO: 34. In some embodiments, the CDRs in the V_{H} or V_{L} chain are as set forth in the combinations provided for herein.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 45; provided that the V_{L} peptide comprises a LCDR1 having a sequence of SEQ ID NO: 35; a LCDR2 having a sequence of SEQ ID NO: 36; and a LCDR3 having a sequence of SEQ ID NO: 37; and the V_{H} peptide comprises a HCDR1 having a sequence of SEQ ID NO: 32; a HCDR2 having a sequence of SEQ ID NO: 33; and a HCDR3 having a sequence of SEQ ID NO: 34.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 45; provided that the V_{L} peptide comprises a LCDR1 having a sequence of SEQ ID NO: 35, wherein the LCDR1 comprises at most 1 conservative amino acid substitution, a LCDR2 having a sequence of SEQ ID NO: 36, wherein the LCDR2 comprises at most 1 conservative amino acid substitution, and a LCDR3 having a sequence of SEQ ID NO: 37, wherein the LCDR3 comprises at most 1 conservative amino acid substitution; and the V_{H} peptide comprises a HCDR1 having a sequence of SEQ ID NO: 32, wherein the HCDR1 comprises at most 1 conservative amino acid substitution, a HCDR2 having a sequence of SEQ ID NO: 33, wherein the HCDR2 comprises at most 1 conservative amino acid substitution, and a HCDR3 having a sequence of SEQ ID NO: 34, wherein the HCDR3 comprises at most 1 conservative amino acid substitution.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 45; provided that the V_{H} peptide and a V_{L} peptide comprise a light chain CDR1 having a sequence of SEQ ID NO: 35; a light chain CDR2 having a sequence of SEQ ID NO: 36; a light chain CDR3 having a sequence of SEQ ID NO: 37; and/or a heavy chain CDR1 having a sequence of SEQ ID NO: 38; a heavy chain CDR2 having a sequence of SEQ ID NO: 39; and a heavy chain CDR3 having a sequence of SEQ ID NO: 34. In some embodiments, the CDRs in the V_{H} or V_{L} chain are as set forth in the combinations provided for herein.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 45; provided that the V_{L} peptide comprises a LCDR1 having a sequence of SEQ ID NO: 35; a LCDR2 having a sequence of SEQ ID NO: 36; and a LCDR3 having a sequence of SEQ ID NO: 37; and the V_{H} peptide comprises a HCDR1 having a sequence of SEQ ID NO: 38; a HCDR2 having a sequence of SEQ ID NO: 39; and a HCDR3 having a sequence of SEQ ID NO: 34.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 45; provided that the V_{L} peptide comprises a LCDR1 having a sequence of SEQ ID NO: 35, wherein the LCDR1 comprises at most 1 conservative amino acid substitution, a LCDR2 having a sequence of SEQ ID NO: 36, wherein the LCDR2 comprises at most 1 conservative amino acid substitution, and a LCDR3 having a sequence of SEQ ID NO: 37, wherein the LCDR3 comprises at most 1 conservative amino acid substitution; and the V_{H} peptide comprises a HCDR1 having a sequence of SEQ ID NO: 38, wherein the HCDR1 comprises at most 1 conservative amino acid substitution, a HCDR2 having a sequence of SEQ ID NO: 39, wherein the HCDR2 comprises at most 1 conservative amino acid substitution, and a HCDR3 having a sequence of SEQ ID NO: 34, wherein the HCDR3 comprises at most 1 conservative amino acid substitution.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 45; provided that the V_{H} peptide and a V_{L} peptide comprise a light chain CDR1 having a sequence of SEQ ID NO: 43; a light chain CDR2 having a sequence of LA; a light chain CDR3 having a sequence of SEQ ID NO: 37; and/or a heavy chain CDR1 having a sequence of SEQ ID NO: 40; a heavy chain CDR2 having a sequence of SEQ ID NO: 41; and a heavy chain CDR3 having a sequence of SEQ ID NO: 42. In some embodiments, the CDRs in the V_{H} or V_{L} chain are as set forth in the combinations provided for herein.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 45; provided that the V_{L} peptide comprises a LCDR1 having a sequence of SEQ ID NO: 43; a LCDR2 having a sequence of LA; and a LCDR3 having a sequence of SEQ ID NO: 37; and the V_{H} peptide comprises a HCDR1 having a sequence of SEQ ID NO: 40; a HCDR2 having a sequence of SEQ ID NO: 41; and a HCDR3 having a sequence of SEQ ID NO: 42.

In some embodiments, a polypeptide comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 44; and the V_{L} peptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 45; provided that the V_{L} peptide comprises a LCDR1 having a sequence of SEQ ID NO: 43, wherein the LCDR1 comprises at most 1 conservative amino acid substitution, a LCDR2 having a sequence of LA, wherein the LCDR2 comprises at most 1 conservative amino acid substitution, and a LCDR3 having a sequence of SEQ ID NO: 37, wherein the LCDR3 comprises at most 1 conservative amino acid substitution; and the V_{H} peptide comprises a HCDR1 having a sequence of SEQ ID NO: 40, wherein the HCDR1 comprises at most 1 conservative amino acid substitution, a HCDR2 having a sequence of SEQ ID NO: 41, wherein the HCDR2 comprises at most 1 conservative amino acid substitution, and a HCDR3 having a sequence of SEQ ID NO: 42, wherein the HCDR3 comprises at most 1 conservative amino acid substitution.

In some embodiments, a polypeptide comprises a V_{H} peptide and a V_{L} peptide, wherein the V_{H} peptide comprises a sequence of SEQ ID NO: 44 and the V_{L} peptide comprises a sequence of SEQ ID NO: 45.

In some embodiments, a polypeptide as provided herein binds to non-human primate CD8. In some embodiments, a polypeptide as provided herein binds to human CD8.

As provided for herein, the different polypeptides (V_{H} or V_{L}) described herein can be linked with a peptide linker or not linked with a peptide linker and instead for a continuous sequence. In some embodiments, the peptide linker comprises a sequence of (GGGGS)ₙ (SEQ ID NO: 26), wherein each n is independently 1-5. In some embodiments, n is 1. In some embodiments, n is 2. In some embodiments, n is 3. In some embodiments, n is 4. In some embodiments, n is 5. The linked peptide format can be represented by a formula of V_{H}-Z-V_{L} or V_{L}-Z-V_{H}, wherein Z is the peptide linker. In some embodiments, Z is (GGGGS)ₙ (SEQ ID NO: 26), wherein each n is independently 1-5.

In some embodiments, a polypeptide comprising the linked peptide represented by a formula of V_{L}-Z-V_{H} comprises a heavy chain variable region as set forth in SEQ ID NO: 44 linked via a linker sequence GGGGSGGGGSGGGGSGGGGS (SEQ ID NO: 27) to a light chain variable region as set forth in SEQ ID NO: 45. In some embodiments, a polypeptide comprising a V_{L} linked via a peptide linker to a V_{H} has the sequence as set forth below:

(SEQ ID NO: 46)
NIVLTQSPASLAVSLGQRATISCRASESVDGFGNSF
MNWYQQKPGQSPKLLIYLASNLESGVPARFSGSGSR
TDFTLTIDPVEADDAATYYCQQNNEDPYTFGGGTKL
EIKRGGGGSGGGGSGGGGSGGGGSEVQLQQSGPELV
KPGASVKISCKASRYTFTDYNLHWVKLSHEKSLEWI
GFIYPYNGGTGYNQKFKNKAKLTVDYSSSTAYMELR
SLTSVDAAVYYCARDHRYNEGVSFDYWGQGTTLTVS
S

In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 46. In some embodiments, a polypeptide comprises a sequence that is at least 90% identical to a sequence of SEQ ID NO: 46. In some embodiments, a polypeptide comprises a sequence that is at least 95% identical to a sequence of SEQ ID NO: 46. In some embodiments, a polypeptide comprises a sequence that is at least 99% identical to a sequence of SEQ ID NO: 46. In some embodiments, a polypeptide comprises a sequence as set forth in SEQ ID NO: 46. In some embodiments, the polypeptide as set forth in SEQ ID NO: 46 is an antibody, or an antigen binding fragment thereof. In some embodiments, the antibody is an anti-CD8 antibody.

In some embodiments, a polypeptide comprising the linked peptide represented by a formula of V_{H}-Z-V_{L} comprises a light chain variable region as set forth in SEQ ID NO: 45 linked via a linker sequence GGGGSGGGGSGGGGSGGGGS (SEQ ID NO: 27) to a heavy chain variable region as set forth in SEQ ID NO: 44. In some embodiments, a polypeptide comprising a V_{H} linked via a peptide linker to a V_{L} has the sequence as set forth below:

(SEQ ID NO: 47)
EVQLQQSGPELVKPGASVKISCKASRYTFTDYNLHW
VKLSHEKSLEWIGFIYPYNGGTGYNQKFKNKAKLTV
DYSSSTAYMELRSLTSVDAAVYYCARDHRYNEGVSF
DYWGQGTTLTVSSGGGGSGGGGSGGGGSGGGGSNIV
LTQSPASLAVSLGQRATISCRASESVDGFGNSFMNW
YQQKPGQSPKLLIYLASNLESGVPARFSGSGSRTDF
TLTIDPVEADDAATYYCQQNNEDPYTFGGGTKLEIK
R

In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 47. In some embodiments, a polypeptide comprises a sequence that is at least 90% identical to a sequence of SEQ ID NO: 47. In some embodiments, a polypeptide comprises a sequence that is at least 95% identical to a sequence of SEQ ID NO: 47. In some embodiments, a polypeptide comprises a sequence that is at least 99% identical to a sequence of SEQ ID NO: 47. In some embodiments, a polypeptide comprises a sequence as set forth in SEQ ID NO: 47. In some embodiments, the polypeptide as set forth in SEQ ID NO: 47 is an antibody, or an antigen binding fragment thereof. In some embodiments, the antibody is an anti-CD8 antibody. In some embodiments, the anti-CD8 antibody binds to non-human primate CD8. In some embodiments, the anti-CD8 antibody binds to human CD8.

In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 46 and comprises an Fc region, such as those provided herein. In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 46 and comprises an Fc region, such as those provided herein, and a transmembrane domain, such as those provided herein. In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 46 and comprises an Fc region, such as those provided herein, and a CD8/CD28 transmembrane domain. In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 46 and comprises an Fc region, such as those provided herein, a transmembrane domain, such as those provided herein, and an Env incorporation motif.

In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 47 and comprises an Fc region, such as those provided herein. In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 47 and comprises an Fc region, such as those provided herein, and a transmembrane domain, such as those provided herein. In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 47 and comprises an Fc region, such as those provided herein, and a CD8/CD28 transmembrane domain. In some embodiments, a polypeptide comprises a sequence that is at least 85%, 86%, 87%, 88%, 89%, 90%, 91%, 92%, 93%, 94%, 95%, 96%, 97%, 98%, or 99% identical to a sequence of SEQ ID NO: 47 and comprises an Fc region, such as those provided herein, a transmembrane domain, such as those provided herein, and an Env incorporation motif.

As provided for herein, the polypeptide, antibodies, or antigen binding fragments thereof can be variants of the sequences.

The sequences of the polypeptides or antibodies can be modified to yield human IgG antibodies. The conversion of the sequences provided herein can be modified to yield other types of antibodies. The CDRs can also be linked to other antibodies, proteins, or molecules to create antibody fragments that bind CD8.

In some embodiments, a polypeptide or an antibody as provided herein is a targeting moiety on the surface of an engineered viral particle. In some embodiments, the targeting moiety allows for binding to a target cell. In some embodiments, the targeting moiety is a CD8 binding moiety, such as a polypeptide or an antibody as provided herein. In some embodiments, the targeting moiety comprises a sequence that is at least 85%, 86%, 87%, 88%, 89%, 90%, 91%, 92%, 93%, 94%, 95%, 96%, 97%, 98%, or 99% identical to a sequence of SEQ ID NO: 46. In some embodiments, the targeting moiety comprises a sequence that is at least 90% identical to a sequence of SEQ ID NO: 46. In some embodiments, the targeting moiety comprises a sequence that is at least 95% identical to a sequence of SEQ ID NO: 46. In some embodiments, the targeting moiety comprises a sequence that is at least 99% identical to a sequence of SEQ ID NO: 46. In some embodiments, the targeting moiety comprises a sequence as set forth in SEQ ID NO: 46. In some embodiments, the targeting moiety as set forth in SEQ ID NO: 46 is an antibody, or an antigen binding fragment thereof. In some embodiments, the targeting moiety is an anti-CD8 antibody.

In some embodiments, a polypeptide or an antibody as provided for herein is a targeting moiety on the surface of an engineered viral particle. In some embodiments, the engineered viral particle is a pseudotyped viral-like particle. In some embodiments, the targeting moiety allows for binding to a target cell. In some embodiments, the targeting moiety is a CD8 binding moiety, such as a polypeptide or an antibody as provided herein. In some embodiments, the targeting moiety comprises a sequence that is at least 85%, 86%, 87%, 88%, 89%, 90%, 91%, 92%, 93%, 94%, 95%, 96%, 97%, 98%, or 99% identical to a sequence of SEQ ID NO: 47. In some embodiments, the targeting moiety comprises a sequence that is at least 90% identical to a sequence of SEQ ID NO: 47. In some embodiments, the targeting moiety comprises a sequence that is at least 95% identical to a sequence of SEQ ID NO: 47. In some embodiments, the targeting moiety comprises a sequence that is at least 99% identical to a sequence of SEQ ID NO: 47. In some embodiments, the targeting moiety comprises a sequence as set forth in SEQ ID NO: 47. In some embodiments, the targeting moiety as set forth in SEQ ID NO: 47 is an antibody, or an antigen binding fragment thereof. In some embodiments, the targeting moiety is an anti-CD8 antibody. In some embodiments, the anti-CD8 antibody binds to non-human primate CD8. In some embodiments, the anti-CD8 antibody binds to human CD8.

In some embodiments, a polypeptide comprises a sequence having a sequence as set forth in SEQ ID NO: 46 and comprises an Fc region, such as those provided herein. In some embodiments, a polypeptide comprises a sequence having a sequence as set forth in SEQ ID NO: 46 and comprises an Fc region, such as those provided herein, and a transmembrane domain, such as those provided herein. In some embodiments, a polypeptide comprises a sequence having a sequence as set forth in SEQ ID NO: 46 and comprises an Fc region, such as those provided herein, and a CD8/CD28 transmembrane domain. In some embodiments, a polypeptide comprises a sequence having a sequence as set forth in SEQ ID NO: 46 and comprises an Fc region, such as those provided herein, a transmembrane domain, such as those provided herein, and an Env incorporation motif.

In some embodiments, a polypeptide comprises a sequence having a sequence as set forth in SEQ ID NO: 47 and comprises an Fc region, such as those provided herein. In some embodiments, a polypeptide comprises a sequence having a sequence as set forth in SEQ ID NO: 47 and comprises an Fc region, such as those provided herein, and a transmembrane domain, such as those provided herein. In some embodiments, a polypeptide comprises a sequence having a sequence as set forth in SEQ ID NO: 47 and comprises an Fc region, such as those provided herein, and a CD8/CD28 transmembrane domain. In some embodiments, a polypeptide comprises a sequence having a sequence as set forth in SEQ ID NO: 70 and comprises an Fc region, such as those provided herein, a transmembrane domain, such as those provided herein, and an Env incorporation motif.

In some embodiments, the viral particle comprising Spring viremia of carp virus G protein and/or a targeting moiety as provided for herein further comprises a gag protein. In some embodiments, the gag protein is not a chimeric gag protein and is a wild-type gag protein. In some embodiments, the gag protein is a chimeric gag protein. In some embodiments, the gag protein is a HIV gag protein. In some embodiments, the chimeric gag protein is a xHIV gag protein. The chimeric gag protein xHIV is described in Uchida et al. J. Virol, October 2009, p. 9854-9862, the contents of which are hereby incorporated by reference in its entirety.

In some embodiments, the chimeric gag protein comprises SIV and HIV sequences. In some embodiments, the chimeric gag protein comprising SIV and HIV sequences is referred to as xHIV gag protein. One non-limiting example of such a SIV/HIV xHIV gag protein may be found in U.S. Pat. No. 11,191,784, which is hereby incorporated by reference in its entirety.

In some embodiments, the xHIV comprises a HIV long-terminal repeat (LTR) a HIV gag protein (gag), a SIV element, a HIV Pol protein (Pol) and a HIV Envelope (Env) protein. In some embodiments, gag, Pol, and/or Env protein is a polyprotein. In some embodiments, the gag protein, the SIV element, and the Pol protein of the xHIV are encoded by the nucleic acid sequence as set forth in SEQ ID NO: 57:

(SEQ ID NO: 57)
atgggtgcgagagcgtcggtattaagcgggggagaattagataaatggg
aaaaaattcggttaaggccagggggaaagaaacaatataaactaaaaca
tatagtatgggcaagcagggagctagaacgattcgcagttaatcctggc
cttttagagacatcagaaggctgtagacaaatactgggacagctacaac
catcccttcagacaggatcagaagaacttagatcattatataatacaat
agcagtcctctattgtgtgcatcaaaggatagatgtaaaagacactaag
gaagccttagataagatagaggaagaacaaaacaaaagtaagaaaaagg
cacagcaagcagcagctgacacaggaaacaacagccaggtcagccaaaa
ttacccagtacaacaaataggtggtaactatgtccacctgccattaagc
ccgagaacattaaatgcctgggtaaaattgatagaggaaaagaaatttg
gagcagaagtagtgccaggatttcaggcactgtcagaaggttgcacccc
ctatgacattaatcagatgttaaattgtgtgggagaccatcaagcggct
atgcagattatcagagatattataaacgaggaggttgcagattgggact
tgcagcacccacaaccagctccacaacaaggacaacttagggagccgtc
aggatcagatattgcaggaacaactagttcagtagatgaacaaatccag
tggatgtacagacaacagaaccccataccagtaggcaacatttacagga
gatggatccaactggggttgcaaaaatgtgtcagaatgtataacccaac
aaacattctagatgtaaaacaagggccaaaagagccatttcagagctat
gtagacaggttctacaaaagtttaagagcagaacagacagatgcagcag
taaagaattggatgactcaaacactgctgattcaaaatgctaacccaga
ttgcaagctagtgctgaaggggttgggaccaggagcgacactagaagaa
atgatgacagcatgtcagggagtggggggacccggccataaagcaagag
ttttggctgaagcaatgagccaagtaacaaatccagctaccataatgat
acagaaaggcaattttaggaaccaaagaaagactgttaagtgtttcaat
tgtggcaaagaagggcacatagccaaaaattgcagggcccctaggaaaa
agggctgttggaaatgtggaaaggaaggacaccaaatgaaagattgtac
tgagagacaggctaattttttagggaagatctggccttcccacaaggga
aggccagggaattttcttcagagcagaccagagccaacagccccaccag
aagagagcttcaggtttggggaagagacaacaactccctctcagaagca
ggagccgatagacaaggaactgtatcctttagcttccctcagatcactc
tttggcagcgacccctcgtcacaataaagataggggggcaattaaagga
agctctattagatacaggagcagatgatacagtattagaagaaatgaat
ttgccaggaagatggaaaccaaaaatgatagggggaattggaggtttta
tcaaagtaagacagtatgatcagatactcatagaaatctgcggacataa
agctataggtacagtattagtaggacctacacctgtcaacataattgga
agaaatctgttgactcagattggctgcactttaaattttcccattagtc
ctattgagactgtaccagtaaaattaaagccaggaatggatggcccaaa
agttaaacaatggccattgacagaagaaaaaataaaagcattagtagaa
atttgtacagaaatggaaaaggaaggaaaaatttcaaaaattgggcctg
aaaatccatacaatactccagtatttgccataaagaaaaaagacagtac
taaatggagaaaattagtagatttcagagaacttaataagagaactcaa
gatttctgggaagttcaattaggaataccacatcctgcagggttaaaac
agaaaaaatcagtaacagtactggatgtgggcgatgcatatttttcagt
tcccttagataaagacttcaggaagtatactgcatttaccatacctagt
ataaacaatgagacaccagggattagatatcagtacaatgtgcttccac
agggatggaaaggatcaccagcaatattccagtgtagcatgacaaaaat
cttagagccttttagaaaacaaaatccagacatagtcatctatcaatac
atggatgatttgtatgtaggatctgacttagaaatagggcagcatagaa
caaaaatagaggaactgagacaacatctgttgaggtggggatttaccac
accagacaaaaaacatcagaaagaacctccattcctttggatgggttat
gaactccatcctgataaatggacagtacagcctatagtgctgccagaaa
aggacagctggactgtcaatgacatacagaaattagtgggaaaattgaa
ttgggcaagtcagatttatgcagggattaaagtaaggcaattatgtaaa
cttcttaggggaaccaaagcactaacagaagtagtaccactaacagaag
aagcagagctagaactggcagaaaacagggagattctaaaagaaccggt
acatggagtgtattatgacccatcaaaagacttaatagcagaaatacag
aagcaggggcaaggccaatggacatatcaaatttatcaagagccattta
aaaatctgaaaacaggaaagtatgcaagaatgaagggtgcccacactaa
tgatgtgaaacaattaacagaggcagtacaaaaaatagccacagaaagc
atagtaatatggggaaagactcctaaatttaaattacccatacaaaagg
aaacatgggaagcatggtggacagagtattggcaagccacctggattcc
tgagtgggagtttgtcaatacccctcccttagtgaagttatggtaccag
ttagagaaagaacccataataggagcagaaactttctatgtagatgggg
cagccaatagggaaactaaattaggaaaagcaggatatgtaactgacag
aggaagacaaaaagttgtccccctaacggacacaacaaatcagaagact
gagttacaagcaattcatctagctttgcaggattcgggattagaagtaa
acatagtgacagactcacaatatgcattgggaatcattcaagcacaacc
agataagagtgaatcagagttagtcagtcaaataatagagcagttaata
aaaaaggaaaaagtctacctggcatgggtaccagcacacaaaggaattg
gaggaaatgaacaagtagataaattggtcagtgctggaatcaggaaagt
actatttttagatggaatagataaggcccaagaagaacatgagaaatat
cacagtaattggagagcaatggctagtgattttaacctaccacctgtag
tagcaaaagaaatagtagccagctgtgataaatgtcagctaaaagggga
agccatgcatggacaagtagactgtagcccaggaatatggcagctagat
tgtacacatttagaaggaaaagttatcttggtagcagttcatgtagcca
gtggatatatagaagcagaagtaattccagcagagacagggcaagaaac
agcatacttcctcttaaaattagcaggaagatggccagtaaaaacagta
catacagacaatggcagcaatttcaccagtactacagttaaggccgcct
gttggtgggcggggatcaagcaggaatttggcattccctacaatcccca
aagtcaaggagtaatagaatctatgaataaagaattaaagaaaattata
ggacaggtaagagatcaggctgaacatcttaagacagcagtacaaatgg
cagtattcatccacaattttaaaagaaaaggggggattggggggtacag
tgcaggggaaagaatagtagacataatagcaacagacatacaaactaaa
gaattacaaaaacaaattacaaaaattcaaaattttcgggtttattaca
gggacagcagagatccagtttggaaaggaccagcaaagctcctctggaa
aggtgaaggggcagtagtaatacaagataatagtgacataaaagtagtg
ccaagaagaaaagcaaagatcatcagggattatggaaaacagatggcag
gtgatgattgtgtggcaagtagacaggatgaggattaa

In some embodiments, the gag protein is encoded by the nucleic acid sequence as set forth in SEQ ID NO: 58:

(SEQ ID NO: 58)
atgggtgcgagagcgtcggtattaagcgggggagaattagataaatggga
aaaaattcggttaaggccagggggaaagaaacaatataaactaaaacata
tagtatgggcaagcagggagctagaacgattcgcagttaatcctggcctt
ttagagacatcagaaggctgtagacaaatactgggacagctacaaccatc
ccttcagacaggatcagaagaacttagatcattatataatacaatagcag
tcctctattgtgtgcatcaaaggatagatgtaaaagacactaaggaagcc
ttagataagatagaggaagaacaaaacaaaagtaagaaaaaggcacagca
agcagcagctgacacaggaaacaacagccaggtcagccaaaattacccag
tacaacaaataggtggtaactatgtccacctgccattaagcccgagaaca
ttaaatgcctgggtaaaattgatagaggaaaagaaatttggagcagaagt
agtgccaggatttcaggcactgtcagaaggttgcaccccctatgacatta
atcagatgttaaattgtgtgggagaccatcaagcggctatgcagattatc
agagatattataaacgaggaggttgcagattgggacttgcagcacccaca
accagctccacaacaaggacaacttagggagccgtcaggatcagatattg
caggaacaactagttcagtagatgaacaaatccagtggatgtacagacaa
cagaaccccataccagtaggcaacatttacaggagatggatccaactggg
gttgcaaaaatgtgtcagaatgtataacccaacaaacattctagatgtaa
aacaagggccaaaagagccatttcagagctatgtagacaggttctacaaa
agtttaagagcagaacagacagatgcagcagtaaagaattggatgactca
aacactgctgattcaaaatgctaacccagattgcaagctagtgctgaagg
ggttgggaccaggagcgacactagaagaaatgatgacagcatgtcaggga
gtggggggacccggccataaagcaagagttttggctgaagcaatgagcca
agtaacaaatccagctaccataatgatacagaaaggcaattttaggaacc
aaagaaagactgttaagtgtttcaattgtggcaaagaagggcacatagcc
aaaaattgcagggcccctaggaaaaagggctgttggaaatgtggaaagga
aggacaccaaatgaaagattgtactgagagacaggctaattttttaggga
agatctggccttcccacaagggaaggccagggaattttcttcagagcaga
ccagagccaacagccccaccagaagagagcttcaggtttggggaagagac
aacaactccctctcagaagcaggagccgatagacaaggaactgtatcctt
tagcttccctcagatcactctttggcagcgacccctcgtcacaataa

In some embodiments, the SIV element is encoded by the nucleic acid sequence as set forth in SEQ ID NO: 59. In some embodiments, the nucleic acid sequence encoding the SIV element is located within the nucleic acid sequence encoding the gag protein as set forth in SEQ ID NO: 58.

(SEQ ID NO: 59)
Aaaattacccagtacaacaaataggtggtaactatgtccacctgccatta
agcccgagaacattaaatgcctgggtaaaattgatagaggaaaagaaatt
tggagcagaagtagtgccaggatttcaggcactgtcagaaggttgcaccc
cctatgacattaatcagatgttaaattgtgtgggagaccatcaagcggct
atgcagattatcagagatattataaacgaggaggttgcagattgggactt
gcagcacccacaaccagctccacaacaaggacaacttagggagccgtcag
gatcagatattgcaggaacaactagttcagtagatgaacaaatccagtgg
atgtacagacaacagaaccccataccagtaggcaacatttacaggagatg
gatccaactggggttgcaaaaatgtgtcagaatgtataacccaacaaaca
ttctagatgtaaaacaagggccaaaagagccatttcagagctatgtagac
aggttctacaaaagtttaagagcagaacagacagatgcagcagtaaagaa
ttggatgactcaaacactgctgattcaaaatgctaacccagattgcaagc
tagtgctgaaggggttggg

In some embodiments, the Pol protein is encoded by the nucleic acid sequence as set forth in SEQ ID NO: 60:

(SEQ ID NO: 60)
tttttagggaagatctggccttcccacaagggaaggccagggaattttc
ttcagagcagaccagagccaacagccccaccagaagagagcttcaggtt
tggggaagagacaacaactccctctcagaagcaggagccgatagacaag
gaactgtatcctttagcttccctcagatcactctttggcagcgacccct
cgtcacaataaagataggggggcaattaaaggaagctctattagataca
ggagcagatgatacagtattagaagaaatgaatttgccaggaagatgga
aaccaaaaatgatagggggaattggaggttttatcaaagtaagacagta
tgatcagatactcatagaaatctgcggacataaagctataggtacagta
ttagtaggacctacacctgtcaacataattggaagaaatctgttgactc
agattggctgcactttaaattttcccattagtcctattgagactgtacc
agtaaaattaaagccaggaatggatggcccaaaagttaaacaatggcca
ttgacagaagaaaaaataaaagcattagtagaaatttgtacagaaatgg
aaaaggaaggaaaaatttcaaaaattgggcctgaaaatccatacaatac
tccagtatttgccataaagaaaaaagacagtactaaatggagaaaatta
gtagatttcagagaacttaataagagaactcaagatttctgggaagttc
aattaggaataccacatcctgcagggttaaaacagaaaaaatcagtaac
agtactggatgtgggcgatgcatatttttcagttcccttagataaagac
ttcaggaagtatactgcatttaccatacctagtataaacaatgagacac
cagggattagatatcagtacaatgtgcttccacagggatggaaaggatc
accagcaatattccagtgtagcatgacaaaaatcttagagccttttaga
aaacaaaatccagacatagtcatctatcaatacatggatgatttgtatg
taggatctgacttagaaatagggcagcatagaacaaaaatagaggaact
gagacaacatctgttgaggtggggatttaccacaccagacaaaaaacat
cagaaagaacctccattcctttggatgggttatgaactccatcctgata
aatggacagtacagcctatagtgctgccagaaaaggacagctggactgt
caatgacatacagaaattagtgggaaaattgaattgggcaagtcagatt
tatgcagggattaaagtaaggcaattatgtaaacttcttaggggaacca
aagcactaacagaagtagtaccactaacagaagaagcagagctagaact
ggcagaaaacagggagattctaaaagaaccggtacatggagtgtattat
gacccatcaaaagacttaatagcagaaatacagaagcaggggcaaggcc
aatggacatatcaaatttatcaagagccatttaaaaatctgaaaacagg
aaagtatgcaagaatgaagggtgcccacactaatgatgtgaaacaatta
acagaggcagtacaaaaaatagccacagaaagcatagtaatatggggaa
agactcctaaatttaaattacccatacaaaaggaaacatgggaagcatg
gtggacagagtattggcaagccacctggattcctgagtgggagtttgtc
aatacccctcccttagtgaagttatggtaccagttagagaaagaaccca
taataggagcagaaactttctatgtagatggggcagccaatagggaaac
taaattaggaaaagcaggatatgtaactgacagaggaagacaaaaagtt
gtccccctaacggacacaacaaatcagaagactgagttacaagcaattc
atctagctttgcaggattcgggattagaagtaaacatagtgacagactc
acaatatgcattgggaatcattcaagcacaaccagataagagtgaatca
gagttagtcagtcaaataatagagcagttaataaaaaaggaaaaagtct
acctggcatgggtaccagcacacaaaggaattggaggaaatgaacaagt
agataaattggtcagtgctggaatcaggaaagtactatttttagatgga
atagataaggcccaagaagaacatgagaaatatcacagtaattggagag
caatggctagtgattttaacctaccacctgtagtagcaaaagaaatagt
agccagctgtgataaatgtcagctaaaaggggaagccatgcatggacaa
gtagactgtagcccaggaatatggcagctagattgtacacatttagaag
gaaaagttatcttggtagcagttcatgtagccagtggatatatagaagc
agaagtaattccagcagagacagggcaagaaacagcatacttcctctta
aaattagcaggaagatggccagtaaaaacagtacatacagacaatggca
gcaatttcaccagtactacagttaaggccgcctgttggtgggcggggat
caagcaggaatttggcattccctacaatccccaaagtcaaggagtaata
gaatctatgaataaagaattaaagaaaattataggacaggtaagagatc
aggctgaacatcttaagacagcagtacaaatggcagtattcatccacaa
ttttaaaagaaaaggggggattggggggtacagtgcaggggaaagaata
gtagacataatagcaacagacatacaaactaaagaattacaaaaacaaa
ttacaaaaattcaaaattttcgggtttattacagggacagcagagatcc
agtttggaaaggaccagcaaagctcctctggaaaggtgaaggggcagta
gtaatacaagataatagtgacataaaagtagtgccaagaagaaaagcaa
agatcatcagggattatggaaaacagatggcaggtgatgattgtgtggc
aagtagacaggatgaggattaa

In some embodiments, the nucleotide sequence encoding the gag protein encodes a protein comprising a sequence as set forth in SEQ ID NO: 61:

(SEQ ID NO: 61)
MGARASVLSGGELDKWEKIRLRPGGKKQYKLKHIVWASRELERFAVNPGL
LETSEGCRQILGQLQPSLQTGSEELRSLYNTIAVLYCVHQRIDVKDTKEA
LDKIEEEQNKSKKKAQQAAADTGNNSQVSQNYPVQQIGGNYVHLPLSPRT
LNAWVKLIEEKKFGAEVVPGFQALSEGCTPYDINQMLNCVGDHQAAMQII
RDIINEEVADWDLQHPQPAPQQGQLREPSGSDIAGTTSSVDEQIQWMYRQ
QNPIPVGNIYRRWIQLGLQKCVRMYNPTNILDVKQGPKEPFQSYVDRFYK
SLRAEQTDAAVKNWMTQTLLIQNANPDCKLVLKGLGPGATLEEMMTACQG
VGGPGHKARVLAEAMSQVTNPATIMIQKGNFRNQRKTVKCFNCGKEGHIA
KNCRAPRKKGCWKCGKEGHQMKDCTERQANFLGKIWPSHKGRPGNFLQSR
PEPTAPPEESFRFGEETTTPSQKQEPIDKELYPLASLRSLFGSDPSSQ

In some embodiments, the nucleotide sequence encoding the Pol protein encodes a protein comprising a sequence as set forth in SEQ ID NO: 62:

(SEQ ID NO: 62)
MNLPGRWKPKMIGGIGGFIKVRQYDQILIEICGHKAIGTVLVGPTPVNI
IGRNLLTQIGCTLNFPISPIETVPVKLKPGMDGPKVKQWPLTEEKIKAL
VEICTEMEKEGKISKIGPENPYNTPVFAIKKKDSTKWRKLVDFRELNKR
TQDFWEVQLGIPHPAGLKQKKSVTVLDVGDAYFSVPLDKDFRKYTAFTI
PSINNETPGIRYQYNVLPQGWKGSPAIFQCSMTKILEPFRKQNPDIVIY
QYMDDLYVGSDLEIGQHRTKIEELRQHLLRWGFTTPDKKHQKEPPFLWM
GYELHPDKWTVQPIVLPEKDSWTVNDIQKLVGKLNWASQIYAGIKVRQL
CKLLRGTKALTEVVPLTEEAELELAENREILKEPVHGVYYDPSKDLIAE
IQKQGQGQWTYQIYQEPFKNLKTGKYARMKGAHTNDVKQLTEAVQKIAT
ESIVIWGKTPKFKLPIQKETWEAWWTEYWQATWIPEWEFVNTPPLVKLW
YQLEKEPIIGAETFYVDGAANRETKLGKAGYVTDRGRQKVVPLTDTTNQ
KTELQAIHLALQDSGLEVNIVTDSQYALGIIQAQPDKSESELVSQIIEQ
LIKKEKVYLAWVPAHKGIGGNEQVDKLVSAGIRKVLFLDGIDKAQEEHE
KYHSNWRAMASDFNLPPVVAKEIVASCDKCQLKGEAMHGQVDCSPGIWQ
LDCTHLEGKVILVAVHVASGYIEAEVIPAETGQETAYFLLKLAGRWPVK
TVHTDNGSNFTSTTVKAACWWAGIKQEFGIPYNPQSQGVIESMNKELKK
IIGQVRDQAEHLKTAVQMAVFIHNFKRKGGIGGYSAGERIVDIIATDIQ
TKELQKQITKIQNFRVYYRDSRDPVWKGPAKLLWKGEGAVVIQDNSDIK
VVPRRKAKIIRDYGKQMAGDDCVASRQDED

In some embodiments, the viral particle comprising Spring viremia of carp virus G protein and/or a targeting moiety as provided for herein comprises a nucleic acid molecule encoding for a heterologous molecule of interest or “cargo.” For example, heterologous molecule of interest is meant to refer to any product that may be encoded by a nucleic acid molecule. As non-limiting examples, “cargo” or “heterologous molecule of interest” may refer to an siRNA, an shRNA, a peptide, a polypeptide, a protein, a viral payload, a viral genome, or a combination thereof. In some embodiments, the polypeptide is a chimeric antigen receptor (“CAR”).

A “chimeric antigen receptor” or “CAR” as used herein refers to an antigen-binding domain that is fused, directly, or indirectly (e.g. via a hinge or transmembrane domain to an intracellular signaling domain capable of activating or stimulating an immune cell. Most commonly, the CAR's extracellular binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). In various embodiments, this scFv is fused to a transmembrane domain and then to an intracellular signaling domain. However, the antigen binding domain can be any molecule that can bind to the to target on the cell. For example, the antigen binding domain of a CAR can be an antibody, a scFv antibody, an antigen binding domain, an ankyrin repeat (e.g. DARPIN), a VHH domain antibody, a nanobody, single domain antibody, a FN3 domain, or any combination thereof. In some embodiments, a CAR includes those that solely provide CD3ζ signals upon antigen binding. In some embodiments, the CAR includes those that provide both costimulation (e.g. CD28 or CD137) and activation (CD3 ζ). In some embodiments, the CARs include those that provide multiple costimulation (e.g. CD28 and CD137) and activation (CD3ζ). In various embodiments, the CAR is selected to have high affinity or avidity for the antigen. In some embodiments, the CAR comprises the 4-1BB domain as well. These are merely illustrative in nature and are not limiting to the present embodiments and any chimeric antigen receptor can be delivered in conjunction with the viral particles and vectors provided for herein. These are non-limiting examples of CARs and any CAR construct could be encoded for by the nucleic acid molecule.

In some embodiments, the antigen-binding domain of the CAR comprises a V_{H} domain, a V_{L} domain, or a V_{H} and a V_{L} domain. In some embodiments, the V_{H} domain comprises an amino acid sequence having at least 75%, 80%, 85%, 85%, 87%, 88%, 89%, 90%, 91%, 92%, 93%, 94%, 95%, 96%, 97%, 98%, 99%, or 100% identity to SEQ ID NO: 48, or any value or range in-between.

(SEQ ID NO: 48)
EVQLVESGGGLVQPGRSLRLSCAASGFTFNDYAMHWVRQAPGKGLEWVST
ISWNSGSIGYADSVKGRFTISRDNAKKSLYLQMNSLRAEDTALYYCAKDI
QYGNYYYGMDVWGQGTTVTVSS

In some embodiments, the V_{H} domain comprises an amino acid sequence having at least 90% identity to SEQ ID NO: 48. In some embodiments, the V_{H} domain comprises an amino acid sequence having at least 95% identity to SEQ ID NO: 48. In some embodiments, the V_{H} domain comprises an amino acid sequence having at least 98% identity to SEQ ID NO: 48. In some embodiments, the V_{H} domain comprises an amino acid sequence having at least 99% identity to SEQ ID NO: 48. In some embodiments, the V_{H} domain comprises an amino acid sequence having the sequence of SEQ ID NO: 48.

In some embodiments, the V_{L} domain comprises an amino acid sequence having at least 75%, 80%, 85%, 85%, 87%, 88%, 89%, 90%, 91%, 92%, 93%, 94%, 95%, 96%, 97%, 98%, 99%, or 100% identity to SEQ ID NO: 49, or any value or range in-between.

(SEQ ID NO: 49)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPITFGQ
GTRLEIK

In some embodiments, the V_{L} domain comprises an amino acid sequence having at least 90% identity to SEQ ID NO: 49. In some embodiments, the V_{L} domain comprises an amino acid sequence having at least 95% identity to SEQ ID NO: 49. In some embodiments, the V_{L} domain comprises an amino acid sequence having at least 98% identity to SEQ ID NO: 49. In some embodiments, the V_{L} domain comprises an amino acid sequence having at least 99% identity to SEQ ID NO: 49. In some embodiments, the V_{L} domain comprises an amino acid sequence having the sequence of SEQ ID NO: 49.

In some embodiments, the antigen-binding domain of the CAR comprises a V_{H} domain and a V_{L} domain. In some embodiments, the V_{H} and V_{L} domain are not linked by a linker peptide. In some embodiments, the V_{H} and V_{L} domain are linked by a linker peptide, such as those as provided for herein, including but not limited to: (GGGGS)ₙ (SEQ ID NO: 26), wherein each n is independently 1-5. In some embodiment n is 1. In some embodiment n is 2. In some embodiment n is 3. In some embodiment n is 4. In some embodiment n is 5.

In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 48, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 49. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 48, and comprises a V_{L} having at least 90% identity to SEQ ID NO: 49. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 48, and comprises a V_{L} having at least 95% identity to SEQ ID NO: 49. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 48, and comprises a V_{L} having at least 98% identity to SEQ ID NO: 49. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 48, and comprises a V_{L} having at least 99% identity to SEQ ID NO: 49. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 48, and comprises a V_{L} having the sequence of SEQ ID NO: 49. In some embodiments, the antigen-binding domain of the CAR comprising a V_{H} domain and a V_{L} domain comprises a V_{H} domain having at least 90% identity to SEQ ID NO: 48, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 49. In some embodiments, the antigen-binding domain of the CAR comprising a V_{H} domain and a V_{L} domain comprises a V_{H} domain having at least 95% identity to SEQ ID NO: 48, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 49. In some embodiments, the antigen-binding domain of the CAR comprising a V_{H} domain and a V_{L} domain comprises a V_{H} domain having at least 98% identity to SEQ ID NO: 48, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 49. In some embodiments, the antigen-binding domain of the CAR comprising a V_{H} domain and a V_{L} domain comprises a V_{H} domain having at least 99% identity to SEQ ID NO: 48, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 49. In some embodiments, the antigen-binding domain of the CAR comprising a V_{H} domain and a V_{L} domain comprises a V_{H} domain having an amino acid sequence of SEQ ID NO: 48, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 49. In some embodiments, the antigen-binding domain of the CAR comprising a V_{H} domain and a V_{L} domain comprises a V_{H} domain having at least 90% identity to SEQ ID NO: 48, and comprises a V_{L} having at least 90% identity to SEQ ID NO: 49. In some embodiments, the antigen-binding domain of the CAR comprising a V_{H} domain and a V_{L} domain comprises a V_{H} domain having at least 95% identity to SEQ ID NO: 48, and comprises a V_{L} having at least 90% identity to SEQ ID NO: 49. In some embodiments, the antigen-binding domain of the CAR comprising a V_{H} domain and a V_{L} domain comprises a V_{H} domain having at least 90% identity to SEQ ID NO: 48, and comprises a V_{L} having at least 95% identity to SEQ ID NO: 49. In some embodiments, the antigen-binding domain of the CAR comprising a V_{H} domain and a V_{L} domain comprises a V_{H} domain having at least 95% identity to SEQ ID NO: 48, and comprises a V_{L} having at least 95% identity to SEQ ID NO: 49. In some embodiments, the antigen-binding domain of the CAR comprising a V_{H} domain and a V_{L} domain comprises a V_{H} domain having at least 98% identity to SEQ ID NO: 48, and comprises a V_{L} having at least 98% identity to SEQ ID NO: 49. In some embodiments, the antigen-binding domain of the CAR comprising a V_{H} domain and a V_{L} domain comprises a V_{H} domain having at least 99% identity to SEQ ID NO: 48, and comprises a V_{L} having at least 99% identity to SEQ ID NO: 49. In some embodiments, the antigen-binding domain of the CAR comprising a V_{H} domain and a V_{L} domain comprises a V_{H} domain having an amino acid sequence of SEQ ID NO: 48, and comprises a V_{L} having an amino acid sequence of SEQ ID NO: 49.

In some embodiments, the antigen-binding domain of the CAR comprises a formula of V_{H}-Z-V_{L}, wherein V_{H} is a heavy chain variable region comprising the amino acid sequence of SEQ ID NO: 48, Z is a linker comprising the amino acid sequence GGGGSGGGGSGGGGS (SEQ ID NO: 50), and V_{L} is a light chain variable region comprising the amino acid sequence of SEQ ID NO: 49. In some embodiments, the antigen-binding domain of the CAR comprising a formula of V_{H}-Z-V_{L} has an amino acid sequence as set forth below:

(SEQ ID NO: 51)
EVQLVESGGGLVQPGRSLRLSCAASGFTFNDYAMHWVRQAPGKGLEWVST
ISWNSGSIGYADSVKGRFTISRDNAKKSLYLQMNSLRAEDTALYYCAKDI
QYGNYYYGMDVWGQGTTVTVSSGGGGSGGGGSGGGGSEIVLTQSPATLSL
SPGERATLSCRASQSVSSYLAWYQQKPGQAPRLLIYDASNRATGIPARFS
GSGSGTDFTLTISSLEPEDFAVYYCQQRSNWPITFGQGTRLEIK

In some embodiments, the antigen-binding domain of the CAR comprises an amino acid sequence having at least 85%, 86%, 87%, 88%, 89%, 90%, 91%, 92%, 93%, 94%, 95%, 96%, 97%, 98%, 99%, or 100% identity to a sequence of SEQ ID NO: 51. In some embodiments, the antigen-binding domain of the CAR comprises an amino acid sequence having at least 90% identity to a sequence of SEQ ID NO: 51. In some embodiments, the antigen-binding domain of the CAR comprises an amino acid sequence having at least 95% identity to a sequence of SEQ ID NO: 51. In some embodiments, the antigen-binding domain of the CAR comprises an amino acid sequence having at least 98% identity to a sequence of SEQ ID NO: 51. In some embodiments, the antigen-binding domain of the CAR comprises an amino acid sequence having at least 99% identity to a sequence of SEQ ID NO: 51. In some embodiments, the antigen-binding domain of the CAR comprises the amino acid sequence of SEQ ID NO: 51.

In some embodiments, the antigen-binding domain of the CAR comprises a formula of V_{L}-Z-V_{H}, wherein V_{L} is a light chain variable region comprising the amino acid sequence of SEQ ID NO: 49, Z is a linker comprising the amino acid sequence GGGGSGGGGSGGGGS (SEQ ID NO: 50), and V_{H} is a light chain variable region comprising the amino acid sequence of SEQ ID NO: 48. In some embodiments, the antigen-binding domain of the CAR comprising a formula of V_{L}-Z-V_{H} has an amino acid sequence as set forth below:

(SEQ ID NO: 52)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPITFGQ
GTRLEIKGGGGSGGGGSGGGGSEVQLVESGGGLVQPGRSLRLSCAASGFT
FNDYAMHWVRQAPGKGLEWVSTISWNSGSIGYADSVKGRFTISRDNAKKS
LYLQMNSLRAEDTALYYCAKDIQYGNYYYGMDVWGQGTTVTVSS

In some embodiments, the antigen-binding domain of the CAR comprises an amino acid sequence having at least 85%, 86%, 87%, 88%, 89%, 90%, 91%, 92%, 93%, 94%, 95%, 96%, 97%, 98%, 99%, or 100% identity to a sequence of SEQ ID NO: 52. In some embodiments, the antigen-binding domain of the CAR comprises an amino acid sequence having at least 90% identity to a sequence of SEQ ID NO: 52. In some embodiments, the antigen-binding domain of the CAR comprises an amino acid sequence having at least 95% identity to a sequence of SEQ ID NO: 52. In some embodiments, the antigen-binding domain of the CAR comprises an amino acid sequence having at least 98% identity to a sequence of SEQ ID NO: 52. In some embodiments, the antigen-binding domain of the CAR comprises an amino acid sequence having at least 99% identity to a sequence of SEQ ID NO: 52. In some embodiments, the antigen-binding domain of the CAR comprises the amino acid sequence of SEQ ID NO: 52.

In some embodiments, the antigen-binding domain of the CAR comprises a V_{H} domain, a V_{L} domain, or a V_{H} and a V_{L} domain. In some embodiments, the V_{H} domain comprises an amino acid sequence having at least 75%, 80%, 85%, 85%, 87%, 88%, 89%, 90%, 91%, 92%, 93%, 94%, 95%, 96%, 97%, 98%, 99%, or 100% identity to SEQ ID NO: 53, or any value or range in-between.

(SEQ ID NO: 53)
DIVLTQSPAILSASPGEKVTMTCRASSSVNYMDWYQKKPGSSPKPWIYAT
SNLASGVPARFSGSGSGTSYSLTISRVEAEDAATYYCQQWSFNPPTFGGG
TKLEIKGSTS

In some embodiments, the V_{H} domain comprises an amino acid sequence having at least 90% identity to SEQ ID NO: 53. In some embodiments, the V_{H} domain comprises an amino acid sequence having at least 95% identity to SEQ ID NO: 53. In some embodiments, the V_{H} domain comprises an amino acid sequence having at least 98% identity to SEQ ID NO: 53. In some embodiments, the V_{H} domain comprises an amino acid sequence having at least 99% identity to SEQ ID NO: 53. In some embodiments, the V_{H} domain comprises an amino acid sequence having the sequence of SEQ ID NO: 53.

In some embodiments, the V_{L} domain comprises an amino acid sequence having at least 75%, 80%, 85%, 85%, 87%, 88%, 89%, 90%, 91%, 92%, 93%, 94%, 95%, 96%, 97%, 98%, 99%, or 100% identity to SEQ ID NO: 54, or any value or range in-between.

(SEQ ID NO: 54)
EVQLQQSGAELVKPGASVKMSCKASGYTFTSYNMHWVKQTPGQGLEWIGA
IYPGNGDTSYNQKFKGKATLTADKSSSTAYMQLSSLTSEDSADYYCARSN
YYGSSYWFFDVWGAGTTVTVSS

In some embodiments, the V_{L} domain comprises an amino acid sequence having at least 90% identity to SEQ ID NO: 54. In some embodiments, the V_{L} domain comprises an amino acid sequence having at least 95% identity to SEQ ID NO: 54. In some embodiments, the V_{L} domain comprises an amino acid sequence having at least 98% identity to SEQ ID NO: 54. In some embodiments, the V_{L} domain comprises an amino acid sequence having at least 99% identity to SEQ ID NO: 54. In some embodiments, the V_{L} domain comprises an amino acid sequence having the sequence of SEQ ID NO: 54.

In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 53, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 54. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 53, and comprises a V_{L} having at least 90% identity to SEQ ID NO: 54. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 53, and comprises a V_{L} having at least 95% identity to SEQ ID NO: 54. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 53, and comprises a V_{L} having at least 98% identity to SEQ ID NO: 54. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 53, and comprises a V_{L} having at least 99% identity to SEQ ID NO: 54. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 53, and comprises a V_{L} having the sequence of SEQ ID NO: 54. In some embodiments, the antigen-binding domain of the CAR comprising a V_{H} domain and a V_{L} domain comprises a V_{H} domain having at least 90% identity to SEQ ID NO: 53, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 54. In some embodiments, the antigen-binding domain of the CAR comprising a V_{H} domain and a V_{L} domain comprises a V_{H} domain having at least 95% identity to SEQ ID NO: 53, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 54. In some embodiments, the antigen-binding domain of the CAR comprising a V_{H} domain and a V_{L} domain comprises a V_{H} domain having at least 98% identity to SEQ ID NO: 53, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 54. In some embodiments, the antigen-binding domain of the CAR comprising a V_{H} domain and a V_{L} domain comprises a V_{H} domain having at least 99% identity to SEQ ID NO: 53, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 54. In some embodiments, the antigen-binding domain of the CAR comprising a V_{H} domain and a V_{L} domain comprises a V_{H} domain having an amino acid sequence of SEQ ID NO: 53, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 54. In some embodiments, the antigen-binding domain of the CAR comprising a V_{H} domain and a V_{L} domain comprises a V_{H} domain having at least 90% identity to SEQ ID NO: 53, and comprises a V_{L} having at least 90% identity to SEQ ID NO: 54. In some embodiments, the antigen-binding domain of the CAR comprising a V_{H} domain and a V_{L} domain comprises a V_{H} domain having at least 95% identity to SEQ ID NO: 53, and comprises a V_{L} having at least 90% identity to SEQ ID NO: 54. In some embodiments, the antigen-binding domain of the CAR comprising a V_{H} domain and a V_{L} domain comprises a V_{H} domain having at least 90% identity to SEQ ID NO: 53, and comprises a V_{L} having at least 95% identity to SEQ ID NO: 54. In some embodiments, the antigen-binding domain of the CAR comprising a V_{H} domain and a V_{L} domain comprises a V_{H} domain having at least 95% identity to SEQ ID NO: 53, and comprises a V_{L} having at least 95% identity to SEQ ID NO: 54. In some embodiments, the antigen-binding domain of the CAR comprising a V_{H} domain and a V_{L} domain comprises a V_{H} domain having at least 98% identity to SEQ ID NO: 53, and comprises a V_{L} having at least 98% identity to SEQ ID NO: 54. In some embodiments, the antigen-binding domain of the CAR comprising a V_{H} domain and a V_{L} domain comprises a V_{H} domain having at least 99% identity to SEQ ID NO: 53, and comprises a V_{L} having at least 99% identity to SEQ ID NO: 54. In some embodiments, the antigen-binding domain of the CAR comprising a V_{H} domain and a V_{L} domain comprises a V_{H} domain having an amino acid sequence of SEQ ID NO: 53, and comprises a V_{L} having an amino acid sequence of SEQ ID NO: 54.

In some embodiments, the antigen-binding domain of the CAR comprises a formula of V_{H}-Z-V_{L}, wherein V_{H} is a heavy chain variable region comprising the amino acid sequence of SEQ ID NO: 53, Z is a linker comprising the amino acid sequence GGGGSGGGGSGGGGS (SEQ ID NO: 50), and V_{L} is a light chain variable region comprising the amino acid sequence of SEQ ID NO: 54. In some embodiments, the antigen-binding domain of the CAR comprising a formula of V_{H}-Z-V_{L} has an amino acid sequence as set forth below:

(SEQ ID NO: 55)
DIVLTQSPAILSASPGEKVTMTCRASSSVNYMDWYQKKPGSSPKPWIYAT
SNLASGVPARFSGSGSGTSYSLTISRVEAEDAATYYCQQWSFNPPTFGGG
TKLEIKGSTSGGGGSGGGGSGGGGSSEVQLQQSGAELVKPGASVKMSCKA
SGYTFTSYNMHWVKQTPGQGLEWIGAIYPGNGDTSYNQKFKGKATLTADK
SSSTAYMQLSSLTSEDSADYYCARSNYYGSSYWFFDVWGAGTTVTVSS

In some embodiments, the antigen-binding domain of the CAR comprises an amino acid sequence having at least 85%, 86%, 87%, 88%, 89%, 90%, 91%, 92%, 93%, 94%, 95%, 96%, 97%, 98%, 99%, or 100% identity to a sequence of SEQ ID NO: 55. In some embodiments, the antigen-binding domain of the CAR comprises an amino acid sequence having at least 90% identity to a sequence of SEQ ID NO: 55. In some embodiments, the antigen-binding domain of the CAR comprises an amino acid sequence having at least 95% identity to a sequence of SEQ ID NO: 55. In some embodiments, the antigen-binding domain of the CAR comprises an amino acid sequence having at least 98% identity to a sequence of SEQ ID NO: 55. In some embodiments, the antigen-binding domain of the CAR comprises an amino acid sequence having at least 99% identity to a sequence of SEQ ID NO: 55. In some embodiments, the antigen-binding domain of the CAR comprises the amino acid sequence of SEQ ID NO: 55.

In some embodiments, the antigen-binding domain of the CAR comprises a formula of V_{L}-Z-V_{H}, wherein V_{L} is a light chain variable region comprising the amino acid sequence of SEQ ID NO: 54, Z is a linker comprising the amino acid sequence GGGGSGGGGSGGGGS (SEQ ID NO: 50), and V_{H} is a light chain variable region comprising the amino acid sequence of SEQ ID NO: 53. In some embodiments, the antigen-binding domain of the CAR comprising a formula of V_{L}-Z-V_{H} has an amino acid sequence as set forth below:

(SEQ ID NO: 56)
SEVQLQQSGAELVKPGASVKMSCKASGYTFTSYNMHWVKQTPGQGLEWIG
AIYPGNGDTSYNQKFKGKATLTADKSSSTAYMQLSSLTSEDSADYYCARS
NYYGSSYWFFDVWGAGTTVTVSSGGGGSGGGGSGGGGSDIVLTQSPAILS
ASPGEKVTMTCRASSSVNYMDWYQKKPGSSPKPWIYATSNLASGVPARFS
GSGSGTSYSLTISRVEAEDAATYYCQQWSFNPPTFGGGTKLEIKGSTS

In some embodiments, the antigen-binding domain of the CAR comprises an amino acid sequence having at least 85%, 86%, 87%, 88%, 89%, 90%, 91%, 92%, 93%, 94%, 95%, 96%, 97%, 98%, 99%, or 100% identity to a sequence of SEQ ID NO: 56. In some embodiments, the antigen-binding domain of the CAR comprises an amino acid sequence having at least 90% identity to a sequence of SEQ ID NO: 56. In some embodiments, the antigen-binding domain of the CAR comprises an amino acid sequence having at least 95% identity to a sequence of SEQ ID NO: 56. In some embodiments, the antigen-binding domain of the CAR comprises an amino acid sequence having at least 98% identity to a sequence of SEQ ID NO: 56. In some embodiments, the antigen-binding domain of the CAR comprises an amino acid sequence having at least 99% identity to a sequence of SEQ ID NO: 56. In some embodiments, the antigen-binding domain of the CAR comprises the amino acid sequence of SEQ ID NO: 56.

In some embodiments, the antigen-binding domain of the CAR comprises rituximab, ocrelizumab, obinutuzumab, ofatumumab, ibritumomab tiuxetan, tositumomab, or ublituximab. In some embodiment, the antigen-binding domain comprises rituximab. In some embodiment, the antigen-binding domain comprises ofatumumab. In some embodiments, the CAR comprises the 4-1BB domain as well. These are merely illustrative in nature and are not limiting to the present embodiments and any chimeric antigen receptor can be delivered in conjunction with the viral particles and vectors provided for herein. These are non-limiting examples of CARs and any CAR construct could be encoded for by the nucleic acid molecule.

In some embodiments, the pseudotyped viral particle further comprises a heterologous nucleic acid molecule encoding a cargo of interest. The nucleic acid molecule may be useful for modulating the expression of a target gene. In some embodiments, the cargo can be used to modulate the activity of a cell or express a protein that is trafficked to the surface of the target cell. Therefore, in some embodiments, the nucleic acid may comprise an siRNA or an shRNA. The nucleic acid may also encode for a cargo of interest. Therefore, in some embodiments, the cargo of interest may comprise a polypeptide or portion thereof, a protein or portion thereof, a chimeric antigen receptor or portion thereof, or a tumor antigen or a portion thereof. In some embodiments, the cargo of interest is an antibody that is produced by the virus, which can then be secreted by the cell that is infected with the virus. The term “protein” can refer to any polypeptide that carries a native function in a cellular environment. Therefore, in some embodiments, the protein encoded by the nucleic acid cargo of interest may comprise an enzyme, a nuclear receptor, a transporter, a ribosomal protein, a membrane bound protein, a cytoplasmic protein, a G-protein coupled receptor, a voltage gated ion channel, a secretory protein, a mitochondria protein, a cytokine, a chimeric antigen receptor, a tumor antigen, or a portion or chimeric species thereof.

Without being bound to any particular theory, the viral particle comprising the Spring viremia of carp virus G protein as provided for herein that comprises a targeting moiety can be used to express the heterologous molecule of interest in the target cell. Thus, for example, the CAR can be expressed in a T cell that is targeted by a viral particle pseudotyped with a Spring viremia of carp virus G protein as provided for herein. Where the T cell is the intended target the viral particle can comprise a targeting moiety that binds to a target on the surface of a T cell, such as, but not limited to CD2, CD3, CD4, CD5, CD7 or CD8. In some embodiments, the target is CD2. In some embodiments, the target is CD3. In some embodiments, the target is CD4. In some embodiments, the target is CD5. In some embodiments, the target is CD6. In some embodiments, the target is CD7. In some embodiments, the target is CD8.

In some embodiments, the pseudotyped viral particle is a recombinant lentivirus. In some embodiments, the recombinant pseudotyped viral particle is replication competent. In some embodiments, the recombinant pseudotyped viral particle is replication incompetent.

In some embodiments, a pharmaceutical composition is provided comprising the envelope pseudotyped viral particles or vectors as provided for herein, i.e. a particle that comprises a Spring viremia of carp virus G protein provided for herein.

In some embodiments, methods of delivering a cargo of interest to a cell are provided. In some embodiments, the methods comprise contacting the cell with the pseudotyped viral-like particles or viral vectors as provided for herein, or a pharmaceutical composition comprising the same.

In some embodiments, methods of delivering a cargo of interest to a cell in a subject are provided. In some embodiments, the methods comprise administering to the subject the pseudotyped viral-like particles or viral vectors as provided for herein, or a pharmaceutical composition comprising the same. In some embodiments, the cargo is a chimeric antigen receptor or as otherwise provided for herein.

In some embodiments, methods for of delivering a chimeric antigen receptor to a T-cell in a subject are provided. In some embodiments, the methods comprising administering to the subject the pseudotyped viral-like particles or viral vectors as provided for herein, or a pharmaceutical composition comprising the same, wherein the pseudotyped viral-like particle or viral vector comprises a heterologous nucleic acid molecule encoding the chimeric antigen receptor.

Also provided herein are nucleic acid molecules encoding a Spring viremia of carp virus G protein as provided for herein.

Methods of making the viral like particles or vectors comprising a Spring viremia of carp virus G protein are also provided. In some embodiments, the methods comprise transfecting or transducing a packaging cell line with the nucleic acid molecules encoding a Spring viremia of carp virus G protein as provided for herein under conditions sufficient to produce the pseudotyped viral-like particles or viral vectors. In some embodiments, the methods comprise transfecting or transducing a packaging cell line with the plurality of nucleic acid molecules provided for herein under conditions sufficient to produce the pseudotyped viral-like particles or viral vectors. In some embodiments, methods further comprise isolating the pseudotyped viral-like particle or viral vector. In some embodiments, the nucleic acid molecules also comprise a nucleic acid molecule encoding a targeting moiety and/or a cargo that is to be delivered by the viral vector that is produced.

In some embodiments, the methods comprise transfecting or transducing a viral packaging cell with a nucleic acid molecule encoding the polypeptide and a nucleic acid molecule encoding the targeting moiety under conditions sufficient to produce the viral particle. In some embodiments, the method comprises transfecting or transducing the cell with a lentiviral transfer genome encoding for a heterologous molecule of interest. A “lentiviral transfer genome” is a construct that comprises the appropriate viral elements, such as inverted terminal repeats that allow for the molecule of interest to be incorporated into the viral particle. In some embodiments, the methods comprise transfecting or transducing the cell with a nucleic acid molecule encoding gag-pol and/or rev. In some embodiments, the cell already expresses gag-pol and/or rev, such as by stable expression. In some embodiments, the cell is a HEK293, such as a HEK293T cell. In some embodiments, the method further comprises culturing the cells for at least 24 or 48 hours to produce virus. In some embodiments, the methods comprise isolating the viral particles from the cell supernatant. Any method can be used to isolate the viral particles, such as, but not limited to, centrifugation.

Also provided for herein are methods of treating cancer in a subject. In some embodiments, the methods comprise administering to the subject the pseudotyped viral-like particles or viral vectors as provided for herein, or a pharmaceutical composition comprising the same, wherein the pseudotyped viral-like particle or viral vector comprises a heterologous nucleic acid molecule encoding the chimeric antigen receptor.

Also provided herein are methods of treating a disease in a subject in need thereof.

In some embodiments, the methods provided include, but are not limited to, methods of treating a disease in a subject in need thereof, comprising administering to the subject the viral particle(s) provided herein to treat the disease.

In certain embodiments, the disease is a cancer. In addition, the compositions provided for herein can be used in methods for the treatment of any condition related to a cancer, such as a cell-mediated immune response against a tumor cell(s), where it is desirable to treat or alleviate the disease. The types of cancers to be treated include, but are not limited to, carcinoma, blastoma, sarcoma, certain leukemia or lymphoid malignancies, benign and malignant tumors, malignancies e.g., sarcomas, carcinomas, and melanomas. Other exemplary cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included. In one embodiment, the cancer is a hematological tumor. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by the methods disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

In certain exemplary embodiments, the compositions provided herein can be used in methods to treat a myeloma, or a condition related to myeloma. Examples of myeloma or conditions related thereto include, without limitation, light chain myeloma, non-secretory myeloma, monoclonal gamopathy of undetermined significance (MGUS), plasmacytoma (e.g., solitary, multiple solitary, extramedullary plasmacytoma), amyloidosis, and multiple myeloma. In some embodiments, methods of treating multiple myeloma are provided. In some embodiments, the multiple myeloma is refractory myeloma. In some embodiments, the multiple myeloma is relapsed myeloma.

In certain exemplary embodiments, the in vivo modified immune cells produced using the compositions provided herein are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In some embodiments, the melanoma is cutaneous melanoma In some embodiments, the melanoma is refractory melanoma. In some embodiments, the melanoma is relapsed melanoma.

In some embodiments, the compositions provided herein are used to treat a sarcoma, or a condition related to sarcoma. Examples of sarcoma or conditions related thereto include, without limitation, angiosarcoma, chondrosarcoma, chordoma, endotheliosarcoma, Ewing's sarcoma, fibrosarcoma, gastrointestinal stromal tumor, leiomyosarcoma, liposarcoma, lymphangiosarcoma, lymphangioendotheliosarcoma, mesothelioma, malignant peripheral nerve sheath tumor, myxosarcoma, osteogenic sarcoma, osteosarcoma, pleomorphic sarcoma, rhabdomyosarcoma, synovioma, synovial sarcoma, and other soft tissue sarcomas. In some embodiments, the sarcoma is synovial sarcoma. In some embodiments, the sarcoma is liposarcoma such as myxoid/round cell liposarcoma, differentiated/dedifferentiated liposarcoma, or pleomorphic liposarcoma. In some embodiments, the sarcoma is myxoid/round cell liposarcoma. In some embodiments, the sarcoma is refractory sarcoma. In some embodiments, the sarcoma is relapsed sarcoma.

In some embodiments, the subject has been treated with a therapeutic agent targeting the disease or condition, e.g. the tumor, prior to administration of the composition. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the composition is administered prophylactically, e.g., to reduce the likelihood of or prevent relapse. In some aspects, the subject has not received prior treatment with another therapeutic agent.

The administration of the compositions may be carried out in any convenient manner known to those of skill in the art. For example, the compositions may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, intraperitoneally, intranasally, intracranially, or intraosseously. In other instances, the compositions is injected directly into a site of a local disease site in the subject, a lymph node, an organ, a tumor, and the like.

For the prevention or treatment of disease, the appropriate dosage may depend on the type of disease to be treated, the severity and course of the disease, whether the composition is administered for preventive or therapeutic purposes, previous therapy, the subject's clinical history and response to the treatment, and the discretion of the attending physician. The composition is, in some embodiments, suitably administered to the subject at one time or over a series of treatments.

In some embodiments, the composition is administered as part of a combination treatment, such as simultaneously with or sequentially with, in any order, another therapeutic intervention, such as an antibody or produced cell or receptor or agent, such as a cytotoxic or therapeutic agent. The composition(s), in some embodiments, is co-administered with one or more additional therapeutic agents or in connection with another therapeutic intervention, either simultaneously or sequentially in any order. In some contexts, the composition is co-administered with another therapy sufficiently close in time such that the composition enhances the effect of one or more additional therapeutic agents, or vice versa. In some embodiments, the composition is administered prior to the one or more additional therapeutic agents. In some embodiments, the composition is administered after the one or more additional therapeutic agents. In some embodiments, the one or more additional agents includes a cytokine, such as IL-2, for example, to enhance persistence. In some embodiments, the methods comprise administration of a chemotherapeutic agent. In some embodiments, the methods do not comprise the administration of a chemotherapeutic agent.

In certain embodiments, the compositions may be administered to a subject in combination with an immune checkpoint antibody (e.g., an anti-PD1, anti-CTLA-4, or anti-PDL1 antibody). For example, viral vectors may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVO®) or an antigen-binding fragment thereof. In certain embodiments, the compositions may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the composition may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the viral vector. In certain embodiments, combination treatment comprising an immune checkpoint modulator may increase the therapeutic efficacy of a therapy comprising a composition as provided herein. The other therapeutic can be administered simultaneously, before, or after the compositions provided herein are administered to the subject.

In certain embodiments, the subject is provided a secondary treatment. Secondary treatments include but are not limited to chemotherapy, radiation, surgery, and medications. In some embodiments, the subject is not provided a secondary treatment.

In some embodiments, the methods are performed without a lymphodepletion step, such as the administration of cyclophosphamide and/or fludarabine.

In some embodiments, the subject can be administered a conditioning therapy after the administration of the compositions to kill certain immune cells that are not transduced with the CAR encoded by the compositions. This can be done by including a selection marker that is encoded by the nucleic acid cargo of interest. In some embodiments, the conditioning therapy comprises administering an effective amount of cyclophosphamide to the subject. In some embodiments, the conditioning therapy comprises administering an effective amount of fludarabine to the subject. In some embodiments, the conditioning therapy comprises administering an effective amount of a combination of cyclophosphamide and fludarabine to the subject.

In some embodiments, a specific dosage regimen of the present disclosure includes a lymphodepletion step after the administration of the composition. In an exemplary embodiment, the lymphodepletion step includes administration of cyclophosphamide and/or fludarabine.

In some embodiments, the lymphodepletion step includes administration of cyclophosphamide at a dose of between about 200 mg/m2/day and about 2000 mg/m2/day (e.g., 200 mg/m2/day, 300 mg/m2/day, or 500 mg/m2/day). In an exemplary embodiment, the dose of cyclophosphamide is about 300 mg/m2/day. In some embodiments, the lymphodepletion step includes administration of fludarabine at a dose of between about 20 mg/m2/day and about 900 mg/m2/day (e.g., 20 mg/m2/day, 25 mg/m2/day, 30 mg/m2/day, or 60 mg/m2/day). In an exemplary embodiment, the dose of fludarabine is about 30 mg/m2/day.

In some embodiment, the lymphodepletion step includes administration of cyclophosphamide at a dose of between about 200 mg/m2/day and about 2000 mg/m2/day (e.g., 200 mg/m2/day, 300 mg/m2/day, or 500 mg/m2/day), and fludarabine at a dose of between about 20 mg/m2/day and about 900 mg/m2/day (e.g., 20 mg/m2/day, 25 mg/m2/day, 30 mg/m2/day, or 60 mg/m2/day). In an exemplary embodiment, the lymphodepletion step includes administration of cyclophosphamide at a dose of about 300 mg/m2/day, and fludarabine at a dose of about 30 mg/m2/day.

In an exemplary embodiment, the dosing of cyclophosphamide is 300 mg/m2/day over three days, and the dosing of fludarabine is 30 mg/m2/day over three days.

It is known in the art that one of the adverse effects of the use of CAR T cells can be the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade>3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, in some embodiments, the methods comprise, following the diagnosis of CRS, appropriate CRS management strategies to mitigate the physiological symptoms of uncontrolled inflammation without dampening the antitumor efficacy of the in vivo generated cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

In some embodiments, methods of treating cancer in a subject in need thereof are provided, the methods comprising administering to the subject any of the compositions, such as the viral particle(s), provided herein.

The compositions disclosed herein can comprise a pharmaceutical composition, and for example include a pharmaceutically acceptable carrier, and/or a pharmaceutical formulation.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the composition, preferably those with activities complementary to the composition, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the composition in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the composition, by multiple bolus administrations of the composition, or by continuous infusion administration of the composition. In some embodiments, the pharmaceutical composition does not include a chemotherapeutic.

Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, and intraperitoneal administration. In some embodiments, the composition is administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the composition in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The embodiments provided for herein can be used for many purposes, since the a pseudotyped virus capable of fusing with a target cell can be used to deliver a gene or other heterologous sequence of interest.

### ENUMERATED EMBODIMENTS

In some embodiments, the following embodiments are provided:

1. A viral particle comprising a targeting moiety and a polypeptide comprising a viral structural protein comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to an amino acid sequence of SEQ ID NO: 1 or SEQ ID NO: 2.
2. The viral particle of embodiment 1, wherein the viral structural protein comprises a polypeptide comprising the amino acid sequence of SEQ ID NO: 1 or SEQ ID NO: 2.
3. The viral particle of embodiments 1 or 2, wherein the viral particle is a pseudotyped lentivirus.
4. The viral particle of any one of embodiments 1-3, wherein the viral particle further comprises a nucleic acid molecule encoding a heterologous molecule of interest.
5. The viral particle of embodiment 4, wherein the heterologous molecule of interest is an siRNA, an shRNA, a non-coding RNA (e.g. a guide RNA for a CRISPR system), a peptide, a polypeptide, a protein, a viral payload, a viral genome, or a combination thereof.
6. The viral particle of embodiments 4 or 5, wherein the heterologous molecule of interest is a chimeric antigen receptor (“CAR”).
7. The viral particle of embodiment 6, wherein the CAR comprises an antigen binding domain having an amino acid sequence having at least 85%. 86%, 87%, 88%, 89%, 90%, 91%, 92%, 93%, 94%, 95%, 96%, 97%, 98%, 99%, or 100% identity to SEQ ID NO: 51.
8. The viral particle of embodiment 7, wherein the CAR comprises an antigen binding domain having an amino acid sequence having at least 90% identity to SEQ ID NO: 51.
9. The viral particle of embodiment 7, wherein the CAR comprises an antigen binding domain having an amino acid sequence having at least 95% identity to SEQ ID NO: 51.
10. The viral particle of embodiment 7, wherein the CAR comprises an antigen binding domain having an amino acid sequence having at least 98% identity to SEQ ID NO: 51.
11. The viral particle of embodiment 7, wherein the CAR comprises an antigen binding domain having an amino acid sequence having at least 99% identity to SEQ ID NO: 51.
12. The viral particle of embodiment 7, wherein the CAR comprises an antigen binding domain having an amino acid sequence having the amino acid sequence of SEQ ID NO: 51.
13. The viral particle of embodiment 6, wherein the CAR comprises an antigen binding domain having an amino acid sequence having at least 85%. 86%, 87%, 88%, 89%, 90%, 91%, 92%, 93%, 94%, 95%, 96%, 97%, 98%, 99%, or 100% identity to SEQ ID NO: 52.
14. The viral particle of embodiment 13, wherein the CAR comprises an antigen binding domain having an amino acid sequence having at least 90% identity to SEQ ID NO: 52.
15. The viral particle of embodiment 13, wherein the CAR comprises an antigen binding domain having an amino acid sequence having at least 95% identity to SEQ ID NO: 52.
16. The viral particle of embodiment 13, wherein the CAR comprises an antigen binding domain having an amino acid sequence having at least 98% identity to SEQ ID NO: 52.
17. The viral particle of embodiment 13, wherein the CAR comprises an antigen binding domain having an amino acid sequence having at least 99% identity to SEQ ID NO: 52.
18. The viral particle of embodiment 13, wherein the CAR comprises an antigen binding domain having an amino acid sequence having the amino acid sequence of SEQ ID NO: 52.
19. The viral particle of any one of embodiments 1-18, wherein the targeting moiety binds to an immune cell, such as a T cell, B cell; NK cell, dendritic cell, neutrophils, macrophages, a cancer cell; or, for example, CD3+ T cell; CD4+ T cell; CD7+ T cell, CD8+ T cell; CD19+ B cell; CD19+ cancer cell; CD20+ B cell; “CD20+ cancer cell, CD30+ lung epithelial cell; CD34+ haematopoietic stem cell; CD105+ endothelial cell; CD105+ haematopoietic stem cell; CD117+ haematopoietic stem cell; CD133+ cancer cell; EpCAM+ cancer cell; GluA2+ neuron; GluA4+ neuron; Haematopoietic stem cell; Hepatocyte; Her2/Neu+ cancer cell; NKG2D+ natural killer cell; SLC1A3+ astrocyte; SLC7A10+ adipocyte.
20. The viral particle of any one of embodiments 1-19, wherein the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsg1); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-1a); Carbonic anhydrase IX (CA1X); Carcinoembryonic antigen (CEA); CCCTC-Binding Factor (Zinc Finger Protein)-Like (BORIS or Brother of the Regulator of lmprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLVl-Tax; Human papilloma virus E6 (HPV E6); Human papilloma virus E7 (HPV E7); Human Telomerase reverse transcriptase (hTERT); IgE; IL13Ra2; ILl lRa; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-1 (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAP1; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1).
21. The viral particle of any one of embodiments 1-20, wherein the targeting moiety is an antibody, a scFv antibody, an antigen binding domain, an ankyrin repeat (e.g., DARPIN), a VHH domain antibody, a nanobody, single domain antibody, a FN3 domain, or any combination thereof.
22. The viral particle of any one of embodiments 1-21, wherein the targeting moiety is attached to the viral surface through
an IgG Fc stalk;
a polypeptide stalk;
an envelope glycoprotein G or H of a virus of the Paramyxoviridae family, such as a morbillivirus, such as Measles virus, or a henipavirus, such as Nipah virus, Cedar virus, or Hendra virus;
a glycoprotein of a virus of the Rhabdoviridae family, such as a vesicular stomatitis New Jersey virus, a vesicular stomatitis Indiana virus, a vesicular stomatitis Alagoas virus, a vesicular stromatitis Maraba virus, a vesicular stomatitis Carajas virus, Parainfluenza virus, Spodoptera frugiperda rhabdovirus isolate Sf G, Drosophila obscura sigmavirus 10A, Wuhan insect virus 7, Perch virus, or Spring viremia of carp virus;
a glycoprotein of a virus of the Filoviridae family, such as Ebola virus; or
a glycoprotein of a virus of the Arenaviridae family, such as Machupo virus.
   23. The viral particle of any one of embodiments 1-22, wherein viral particle comprises an IgG Fc stalk linked to a targeting moiety, wherein, for example, the IgG Fc stalk is attached to the viral surface.
   24. The viral particle of embodiments 22 or 23, wherein the IgG Fc stalk comprises a transmembrane domain, such as, but not limited to a CD28 transmembrane domain.
   25. The viral particle of embodiment 22, wherein the targeting moiety is attached to the viral surface through a polypeptide stalk.
   26. The viral particle of any one of embodiments 1-25, wherein the targeting moiety binds to CD7.
   27. The viral particle of embodiment 26, wherein the targeting moiety that binds to CD7 comprises a polypeptide comprising: (i) a heavy chain variable region comprising heavy chain CDR1, CDR2, and CDR3 sequences, wherein the heavy chain CDR1 sequence has the amino acid sequence of SEQ ID NO: 12; the heavy chain CDR2 has the amino acid sequence of SEQ ID NO: 13; and the heavy chain CDR3 sequence has the amino acid sequence of SEQ ID NO: 14, or variants of any of the foregoing; and (ii) a light chain variable region comprising light chain CDR1, CDR2, and CDR3 sequences, wherein the light chain CDR1 sequence has the amino acid sequence of SEQ ID NO: 15; the light chain CDR2 sequence has the amino acid sequence of SEQ ID NO: 16; and the light chain CDR3 sequence has the amino acid sequence of SEQ ID NO: 17; or variants of any of the foregoing.
   28. The viral particle of embodiment 27, wherein the heavy chain comprises a heavy chain variable region having at least 90% sequence identity to an amino acid sequence of SEQ ID NO: 24, wherein the polypeptide maintains the amino acid sequences of HCDR1 as set forth in SEQ ID NO: 12; HCDR2 as set forth in SEQ ID NO: 13; and HCDR3 as set forth in SEQ ID NO: 14.
   29. The viral particle of embodiment 27 or embodiment 28, wherein the light chain comprises: a light chain variable region having at least 90% sequence identity to an amino acid sequence of SEQ ID NO: 25, wherein the polypeptide maintains the amino acid sequences of LCDR1 as set forth in SEQ ID NO: 15; LCDR2 as set forth in SEQ ID NO: 16; and LCDR3 as set forth in SEQ ID NO: 17.
   30. The viral particle of any one of embodiments 27-29, wherein the polypeptide comprises a heavy chain and a light chain comprising: a heavy chain variable region of the heavy chain having at least 90% sequence identity to an amino acid sequence of SEQ ID NO: 24, and a light chain variable region of the light chain having at least 90% sequence identity to an amino acid sequence of SEQ ID NO: 25, wherein polypeptide maintains the amino acid sequences of HCDR1 as set forth in SEQ ID NO: 12; HCDR2 as set forth in SEQ ID NO: 13; HCDR3 as set forth in SEQ ID NO: 14; LCDR1 as set forth in SEQ ID NO: 15; LCD2 as set forth in SEQ ID NO: 16; and LCDR3 as set forth in SEQ ID NO: 17.
   31. The viral particle of any one of embodiments 27-30, wherein the light chain and a heavy chain comprise: a heavy chain variable region of the heavy chain having at least 90% sequence identity to an amino acid sequence of SEQ ID NO: 24; and a light chain variable region of the light chain having at least 90% sequence identity to an amino acid sequence of SEQ ID NO: 25.
   32. The viral particle of any one of embodiments 27-31, wherein the light chain and a heavy chain comprise: a heavy chain variable region of the heavy chain having at least 95% sequence identity to an amino acid sequence of SEQ ID NO: 24; and a light chain variable region of the light chain having at least 95% sequence identity to an amino acid sequence of SEQ ID NO: 25.
   33. The viral particle of any one of embodiments 27-32, wherein the light chain and a heavy chain comprise: a heavy chain variable region of the heavy chain having at least 99% sequence identity to an amino acid sequence of SEQ ID NO: 24; and a light chain variable region of the light chain having at least 99% sequence identity to an amino acid sequence of SEQ ID NO: 25.
   34. The viral particle of any one of embodiments 27-33, wherein the light chain and a heavy chain comprise: a heavy chain variable region comprising an amino acid sequence of SEQ ID NO: 24, and a light chain variable region comprising an amino acid sequence of SEQ ID NO: 25.
   35. The viral particle of any one of embodiments 27-34, wherein the heavy chain variable region and the light chain variable region are, or are not, linked by a linker, such as a peptide linker, which can be for example, a glycine/serine linker.
   36. The viral particle of embodiment 35, wherein the peptide linker comprises a sequence of (GGGGS)ₙ(SEQ ID NO: 26), wherein n is independently 1-5.
   37. The viral particle of any one of embodiments 26-36, wherein the targeting moiety that binds to CD7 comprises a polypeptide comprising a sequence having at least 90% sequence identity to an amino acid sequence of SEQ ID NO: 28, at least 95% sequence identity to an amino acid sequence of SEQ ID NO: 28, at least 99% sequence identity to an amino acid sequence of SEQ ID NO: 28, or an amino acid sequence as set forth in SEQ ID NO: 28.
   38. The viral particle of any one of embodiments 26-36, wherein the targeting moiety that binds to CD7 comprises a polypeptide comprising a sequence having at least 90% sequence identity to an amino acid sequence of SEQ ID NO: 29, having at least 95% sequence identity to an amino acid sequence of SEQ ID NO: 29, having at least 99% sequence identity to an amino acid sequence of SEQ ID NO: 29, or an amino acid sequence as set forth in SEQ ID NO: 29.
   39. The viral particle of any one of embodiments 1-25, wherein the targeting moiety binds to CD8.
   40. The viral particle of embodiment 39, wherein the targeting moiety that binds to CD8 comprises a polypeptide comprising: (i) a heavy chain variable region comprising heavy chain CDR1, CDR2, and CDR3 sequences, wherein the heavy chain CDR1 sequence has the amino acid sequence of SEQ ID NO: 32; the heavy chain CDR2 has the amino acid sequence of SEQ ID NO: 33; and the heavy chain CDR3 sequence has the amino acid sequence of SEQ ID NO: 34 or variants of any of the foregoing; and (ii) a light chain variable region comprising light chain CDR1, CDR2, and CDR3 sequences, wherein the light chain CDR1 sequence has the amino acid sequence of SEQ ID NO: 35; the light chain CDR2 sequence has the amino acid sequence of SEQ ID NO: 36; and the light chain CDR3 sequence has the amino acid sequence of SEQ ID NO: 37; or variants of any of the foregoing.
   41. The viral particle of embodiment 40, wherein the heavy chain comprises a heavy chain variable region having at least 90% sequence identity to an amino acid sequence of SEQ ID NO: 44, wherein the polypeptide maintains the amino acid sequences of HCDR1 as set forth in SEQ ID NO: 32; HCDR2 as set forth in SEQ ID NO: 33; and HCDR3 as set forth in SEQ ID NO: 34.
   42. The viral particle of embodiment 40 or embodiment 41, wherein the light chain comprises: a light chain variable region having at least 90% sequence identity to an amino acid sequence of SEQ ID NO: 45, wherein the polypeptide maintains the amino acid sequences of LCDR1 as set forth in SEQ ID NO: 35; LCDR2 as set forth in SEQ ID NO: 36; and LCDR3 as set forth in SEQ ID NO: 37.
   43. The viral particle of any one of embodiments 40-42, wherein the polypeptide comprises a heavy chain and a light chain comprising: a heavy chain variable region of the heavy chain having at least 90% sequence identity to an amino acid sequence of SEQ ID NO: 44, and a light chain variable region of the light chain having at least 90% sequence identity to an amino acid sequence of SEQ ID NO: 45, wherein polypeptide maintains the amino acid sequences of HCDR1 as set forth in SEQ ID NO: 32; HCDR2 as set forth in SEQ ID NO: 33; HCDR3 as set forth in SEQ ID NO: 34; LCDR1 as set forth in SEQ ID NO: 35; LCD2 as set forth in SEQ ID NO: 36; and LCDR3 as set forth in SEQ ID NO: 37.
   44. The viral particle of any one of embodiments 40-43, wherein the light chain and a heavy chain comprise: a heavy chain variable region of the heavy chain having at least 90% sequence identity to an amino acid sequence of SEQ ID NO: 44; and a light chain variable region of the light chain having at least 90% sequence identity to an amino acid sequence of SEQ ID NO: 45.
   45. The viral particle of any one of embodiments 40-44, wherein the light chain and a heavy chain comprise: a heavy chain variable region of the heavy chain having at least 95% sequence identity to an amino acid sequence of SEQ ID NO: 44; and a light chain variable region of the light chain having at least 95% sequence identity to an amino acid sequence of SEQ ID NO: 45.
   46. The viral particle of any one of embodiments 40-45, wherein the light chain and a heavy chain comprise: a heavy chain variable region of the heavy chain having at least 99% sequence identity to an amino acid sequence of SEQ ID NO: 44; and a light chain variable region of the light chain having at least 99% sequence identity to an amino acid sequence of SEQ ID NO: 45.
   47. The viral particle of any one of embodiments 40-46, wherein the light chain and a heavy chain comprise: a heavy chain variable region comprising an amino acid sequence of SEQ ID NO: 44, and a light chain variable region comprising an amino acid sequence of SEQ ID NO: 45.
   48. The viral particle of any one of embodiments 40-47, wherein the heavy chain variable region and the light chain variable region are, or are not, linked by a linker, such as a peptide linker, which can be for example, a glycine/serine linker.
   49. The viral particle of embodiment 48, wherein the peptide linker comprises a sequence of (GGGGS)ₙ(SEQ ID NO: 26), wherein n is independently 1-5.
   50. The viral particle of any one of embodiments 39-49, wherein the targeting moiety that binds to CD8 comprises a polypeptide comprising a sequence having at least 90% sequence identity to an amino acid sequence of SEQ ID NO: 46, at least 95% sequence identity to an amino acid sequence of SEQ ID NO: 46, at least 99% sequence identity to an amino acid sequence of SEQ ID NO: 46, or an amino acid sequence as set forth in SEQ ID NO: 46.
   51. The viral particle of any one of embodiments 39-49, wherein the targeting moiety that binds to CD8 comprises a polypeptide comprising a sequence having at least 90% sequence identity to an amino acid sequence of SEQ ID NO: 47, having at least 95% sequence identity to an amino acid sequence of SEQ ID NO: 47, having at least 99% sequence identity to an amino acid sequence of SEQ ID NO: 47, or an amino acid sequence as set forth in SEQ ID NO: 47.
   52. A method of making the viral particle of any one of embodiments 1-51, the method comprising transfecting or transducing a viral packaging cell with a nucleic acid molecule encoding the polypeptide and a nucleic acid molecule encoding the targeting moiety under conditions sufficient to produce the viral particle.
   53. The method of embodiment 52, wherein the method further comprises transfecting or transducing the cell with a lentiviral transfer genome encoding for a heterologous molecule of interest.
   54. The method of embodiment 53, wherein the wherein the heterologous molecule of interest is an siRNA, an shRNA, a non-coding RNA (e.g. a guide RNA for a CRISPR system), a peptide, a polypeptide, a protein, a viral payload, a viral genome, or a combination thereof.
   55. The method of embodiment 54, wherein the heterologous molecule of interest is a chimeric antigen receptor.
   56. The method of embodiment 55, wherein the chimeric antigen receptor comprises an antigen binding domain having an amino acid sequence having at least 85%. 86%, 87%, 88%, 89%, 90%, 91%, 92%, 93%, 94%, 95%, 96%, 97%, 98%, 99%, or 100% identity to an amino acid sequence of SEQ ID NO: 51.
   57. The method of embodiment 55, wherein the chimeric antigen receptor comprises an antigen binding domain having an amino acid sequence having at least 85%. 86%, 87%, 88%, 89%, 90%, 91%, 92%, 93%, 94%, 95%, 96%, 97%, 98%, 99%, or 100% identity to an amino acid sequence of SEQ ID NO: 52.
   58. The method of any one of embodiments 52-57, wherein the method further comprises transfecting or transducing the cell with a nucleic acid molecule encoding gag-pol and/or rev.
   59. The method of embodiment 52, wherein the cell expresses gag-pol and/or rev.
   60. The method of any one of embodiments 52-59, wherein the cell is a HEK293, such as a HEK293T cell.
   61. The method of any one of embodiments 52-60, wherein the method further comprises culturing the cells for at least 24 or 48 hours to produce virus.
   62. The method of embodiment 61, wherein the method further comprises isolating the viral particles from the cell supernatant, such as, but not limited to, centrifugation.
   63. A method of delivering a heterologous molecule to a target cell, the method comprising contacting the cell with a viral particle of any one of embodiments 4-51.
   64. The method of embodiment 63, wherein the target cell is an immune cell, such as a T cell, B cell; NK cell, dendritic cell, neutrophils, macrophages, a cancer cell; or, for example, CD3+ T cell; CD4+ T cell; CD7+ T cell, CD8+ T cell; CD19+ B cell; CD19+ cancer cell; CD20+ B cell; CD30+ lung epithelial cell; CD34+ haematopoietic stem cell; CD105+ endothelial cell; CD105+ haematopoietic stem cell; CD117+ haematopoietic stem cell; CD133+ cancer cell; EpCAM+ cancer cell; GluA2+ neuron; GluA4+ neuron; Haematopoietic stem cell; Hepatocyte; Her2/Neu+ cancer cell; NKG2D+ natural killer cell; SLC1A3+ astrocyte; SLC7A10+ adipocyte.
   65. The method of embodiments 63 or 64, wherein the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsg1); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-1a); Carbonic anhydrase IX (CA1X); Carcinoembryonic antigen (CEA); CCCTC-Binding Factor (Zinc Finger Protein)-Like (BORIS or Brother of the Regulator of lmprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLVl-Tax; Human papilloma virus E6 (HPV E6); Human papilloma virus E7 (HPV E7); Human Telomerase reverse transcriptase (hTERT); IgE; IL13Ra2; ILl lRa; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-1 (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAP1; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1) on the target cell.
   66. The method of any one of embodiments 63-65, wherein the targeting moiety binds to CD7.
   67. The method of embodiment 66, wherein the targeting moiety that binds to CD7 comprises a polypeptide comprising a sequence having at least 90% sequence identity to an amino acid sequence of SEQ ID NO: 28, at least 95% sequence identity to an amino acid sequence of SEQ ID NO: 28, at least 99% sequence identity to an amino acid sequence of SEQ ID NO: 28, or an amino acid sequence as set forth in SEQ ID NO: 28.
   68. The method of embodiment 66, wherein the targeting moiety that binds to CD7 comprises a polypeptide comprising a sequence having at least 90% sequence identity to an amino acid sequence of SEQ ID NO: 29, at least 95% sequence identity to an amino acid sequence of SEQ ID NO: 29, at least 99% sequence identity to an amino acid sequence of SEQ ID NO: 29, or an amino acid sequence as set forth in SEQ ID NO: 29.
   69. The method of any one of embodiments 63-65, wherein the targeting moiety binds to CD8.
   70. The method of embodiment 69, wherein the targeting moiety that binds to CD8 comprises a polypeptide comprising a sequence having at least 90% sequence identity to an amino acid sequence of SEQ ID NO: 46, at least 95% sequence identity to an amino acid sequence of SEQ ID NO: 46, at least 99% sequence identity to an amino acid sequence of SEQ ID NO: 46, or an amino acid sequence as set forth in SEQ ID NO: 46.
   71. The method of embodiment 69, wherein the targeting moiety that binds to CD8 comprises a polypeptide comprising a sequence having at least 90% sequence identity to an amino acid sequence of SEQ ID NO: 47, at least 95% sequence identity to an amino acid sequence of SEQ ID NO: 47, at least 99% sequence identity to an amino acid sequence of SEQ ID NO: 47, or an amino acid sequence as set forth in SEQ ID NO: 47.
   72. A method of delivering a heterologous molecule to a target cell in a subject, the method comprising administering to the subject a viral particle of any one of embodiments 4-51.
   73. The method of embodiment 72, wherein the target cell is an immune cell, such as a T cell, B cell; NK cell, dendritic cell, neutrophils, macrophages, a cancer cell; or, for example, CD3+ T cell; CD4+ T cell; CD7+ T cell, CD8+ T cell; CD19+ B cell; CD19+ cancer cell; CD20+ B cell; CD20+ cancer cell; CD30+ lung epithelial cell; CD34+ haematopoietic stem cell; CD105+ endothelial cell; CD105+ haematopoietic stem cell; CD117+ haematopoietic stem cell; CD133+ cancer cell; EpCAM+ cancer cell; GluA2+ neuron; GluA4+ neuron; Haematopoietic stem cell; Hepatocyte; Her2/Neu+ cancer cell; NKG2D+ natural killer cell; SLC1A3+ astrocyte; SLC7A10+ adipocyte.
   74. The method of embodiments 72 or 73, wherein the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsg1); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-1a); Carbonic anhydrase IX (CA1X); Carcinoembryonic antigen (CEA); CCCTC-Binding Factor (Zinc Finger Protein)-Like (BORIS or Brother of the Regulator of lmprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLVl-Tax; Human papilloma virus E6 (HPV E6); Human papilloma virus E7 (HPV E7); Human Telomerase reverse transcriptase (hTERT); IgE; IL13Ra2; ILl lRa; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-1 (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAP1; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1) on the target cell.
   75. The method of any one of embodiments 72-74, wherein the targeting moiety binds to CD7.
   76. The method of embodiment 75, wherein the targeting moiety that binds to CD7 comprises a polypeptide comprising a sequence having at least 90% sequence identity to an amino acid sequence of SEQ ID NO: 28, at least 95% sequence identity to an amino acid sequence of SEQ ID NO: 28, at least 99% sequence identity to an amino acid sequence of SEQ ID NO: 28, or an amino acid sequence as set forth in SEQ ID NO: 28.
   77. The method of embodiment 75, wherein the targeting moiety that binds to CD7 comprises a polypeptide comprising a sequence having at least 90% sequence identity to an amino acid sequence of SEQ ID NO: 29, at least 95% sequence identity to an amino acid sequence of SEQ ID NO: 29, at least 99% sequence identity to an amino acid sequence of SEQ ID NO: 29, or an amino acid sequence as set forth in SEQ ID NO: 29.
   78. The method of any one of embodiments 72-74, wherein the targeting moiety binds to CD8.
   79. The method of embodiment 78, wherein the targeting moiety that binds to CD8 comprises a polypeptide comprising a sequence having at least 90% sequence identity to an amino acid sequence of SEQ ID NO: 46, at least 95% sequence identity to an amino acid sequence of SEQ ID NO: 46, at least 99% sequence identity to an amino acid sequence of SEQ ID NO: 46, or an amino acid sequence as set forth in SEQ ID NO: 46.
   80. The method of embodiment 78, wherein the targeting moiety that binds to CD8 comprises a polypeptide comprising a sequence having at least 90% sequence identity to an amino acid sequence of SEQ ID NO: 47, at least 95% sequence identity to an amino acid sequence of SEQ ID NO: 47, at least 99% sequence identity to an amino acid sequence of SEQ ID NO: 47, or an amino acid sequence as set forth in SEQ ID NO: 47.
   81. The method of any one of embodiments 72-80, wherein the heterologous molecule is an siRNA, an shRNA, a non-coding RNA (e.g. a guide RNA for a CRISPR system), a peptide, a polypeptide, a protein, a viral payload, a viral genome, or a combination thereof, such as a chimeric antigen receptor (“CAR”).
   82. The method of embodiment 81, wherein the chimeric antigen receptor comprises an antigen binding domain having an amino acid sequence having at least 85%. 86%, 87%, 88%, 89%, 90%, 91%, 92%, 93%, 94%, 95%, 96%, 97%, 98%, 99%, or 100% identity to an amino acid sequence of SEQ ID NO: 51.
   83. The method of embodiment 81, wherein the chimeric antigen receptor comprises an antigen binding domain having an amino acid sequence having at least 85%. 86%, 87%, 88%, 89%, 90%, 91%, 92%, 93%, 94%, 95%, 96%, 97%, 98%, 99%, or 100% identity to an amino acid sequence of SEQ ID NO: 52.
   84. A method of treating cancer in a subject, the method comprising administering to the subject a viral particle of any one of embodiments 4-51.
   85. The method of embodiment 84, wherein the heterologous molecule is a chimeric antigen receptor.
   86. The method of embodiment 85, wherein the chimeric antigen receptor comprises an antigen binding domain having an amino acid sequence having at least 85%. 86%, 87%, 88%, 89%, 90%, 91%, 92%, 93%, 94%, 95%, 96%, 97%, 98%, 99%, or 100% identity to an amino acid sequence of SEQ ID NO: 51.
   87. The method of embodiment 85, wherein the chimeric antigen receptor comprises an antigen binding domain having an amino acid sequence having at least 85%. 86%, 87%, 88%, 89%, 90%, 91%, 92%, 93%, 94%, 95%, 96%, 97%, 98%, 99%, or 100% identity to an amino acid sequence of SEQ ID NO: 52.
   88. The method of any one of embodiments 84-87, wherein the target cell is an immune cell, such as a T cell, B cell; NK cell, dendritic cell, neutrophils, macrophages, a cancer cell; or, for example, CD3+ T cell; CD4+ T cell; CD7+ T cell, CD8+ T cell; CD19+ B cell; CD19+ cancer cell; CD20+ B cell; CD30+ lung epithelial cell; CD34+ haematopoietic stem cell; CD105+ endothelial cell; CD105+ haematopoietic stem cell; CD117+ haematopoietic stem cell; CD133+ cancer cell; EpCAM+ cancer cell; GluA2+ neuron; GluA4+ neuron; Haematopoietic stem cell; Hepatocyte; Her2/Neu+ cancer cell; NKG2D+ natural killer cell; SLC1A3+ astrocyte; SLC7A10+ adipocyte.
   89. The method of any one of embodiments 84-88, wherein the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsg1); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-1a); Carbonic anhydrase IX (CA1X); Carcinoembryonic antigen (CEA); CCCTC-Binding Factor (Zinc Finger Protein)-Like (BORIS or Brother of the Regulator of lmprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLVl-Tax; Human papilloma virus E6 (HPV E6); Human papilloma virus E7 (HPV E7); Human Telomerase reverse transcriptase (hTERT); IgE; IL13Ra2; ILl lRa; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-1 (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAP1; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1) on the target cell.
   90. The method of any one of embodiments 84-89, wherein the targeting moiety binds to CD7.
   91. The method of embodiment 90, wherein the targeting moiety that binds to CD7 comprises a polypeptide comprising a sequence having at least 90% sequence identity to an amino acid sequence of SEQ ID NO: 28, at least 95% sequence identity to an amino acid sequence of SEQ ID NO: 28, at least 99% sequence identity to an amino acid sequence of SEQ ID NO: 28, or an amino acid sequence as set forth in SEQ ID NO: 28.
   92. The method of embodiment 90, wherein the targeting moiety that binds to CD7 comprises a polypeptide comprising a sequence having at least 90% sequence identity to an amino acid sequence of SEQ ID NO: 29, at least 95% sequence identity to an amino acid sequence of SEQ ID NO: 29, at least 99% sequence identity to an amino acid sequence of SEQ ID NO: 29, or an amino acid sequence as set forth in SEQ ID NO: 29.
   93. The method of any one of embodiments 84-89, wherein the targeting moiety binds to CD8.
   94. The method of embodiment 93, wherein the targeting moiety that binds to CD8 comprises a polypeptide comprising a sequence having at least 90% sequence identity to an amino acid sequence of SEQ ID NO: 46, at least 95% sequence identity to an amino acid sequence of SEQ ID NO: 46, at least 99% sequence identity to an amino acid sequence of SEQ ID NO: 46, or an amino acid sequence as set forth in SEQ ID NO: 46.
   95. The method of embodiment 93, wherein the targeting moiety that binds to CD8 comprises a polypeptide comprising a sequence having at least 90% sequence identity to an amino acid sequence of SEQ ID NO: 47, at least 95% sequence identity to an amino acid sequence of SEQ ID NO: 47, at least 99% sequence identity to an amino acid sequence of SEQ ID NO: 47, or an amino acid sequence as set forth in SEQ ID NO: 47.
   96. The method of any one of embodiments 84-95, wherein the cancer is a cancer as provided for herein, such as a T cell or B cell disorder.
   97. A viral particle comprising a targeting moiety, a polypeptide comprising a viral structural protein, and a nucleic acid molecule encoding a heterologous molecule of interest;
wherein the targeting moiety comprises an amino acid sequence having at least 90% identity to an amino acid sequence of SEQ ID NO: 28, SEQ ID NO: 29, SEQ ID NO: 46, or SEQ ID NO: 47;
wherein the polypeptide comprising a viral structural protein comprises an amino acid sequence having at least 90% identity to an amino acid sequence of SEQ ID NO: 1 or SEQ ID NO: 2;
wherein the nucleic acid molecule encoding a heterologous molecule of interest encodes for a chimeric antigen receptor; and
wherein the chimeric antigen receptor comprises an antigen binding domain having an amino acid sequence having at least 90% identity to an amino acid sequence of SEQ ID NO: 51, SEQ ID NO: 52, SEQ ID NO: 55, or SEQ ID NO: 56.
   98. A viral particle comprising a targeting moiety, a polypeptide comprising a viral structural protein, and a nucleic acid molecule encoding a heterologous molecule of interest;
wherein the targeting moiety comprises an amino acid sequence having at least 95% identity to an amino acid sequence of SEQ ID NO: 28, SEQ ID NO: 29, SEQ ID NO: 46, or SEQ ID NO: 47;
wherein the polypeptide comprising a viral structural protein comprises an amino acid sequence having at least 95% identity to an amino acid sequence of SEQ ID NO: 1 or SEQ ID NO: 2;
wherein the nucleic acid molecule encoding a heterologous molecule of interest encodes for a chimeric antigen receptor; and
wherein the chimeric antigen receptor comprises an antigen binding domain having an amino acid sequence having at least 95% identity to an amino acid sequence of SEQ ID NO: 51, SEQ ID NO: 52, SEQ ID NO: 55, or SEQ ID NO: 56.
   99. A viral particle comprising a targeting moiety, a polypeptide comprising a viral structural protein, and a nucleic acid molecule encoding a heterologous molecule of interest;
wherein the targeting moiety comprises an amino acid sequence having at least 98% identity to an amino acid sequence of SEQ ID NO: 28, SEQ ID NO: 29, SEQ ID NO: 46, or SEQ ID NO: 47;
wherein the polypeptide comprising a viral structural protein comprises an amino acid sequence having at least 98% identity to an amino acid sequence of SEQ ID NO: 1 or SEQ ID NO: 2;
wherein the nucleic acid molecule encoding a heterologous molecule of interest encodes for a chimeric antigen receptor; and
wherein the chimeric antigen receptor comprises an antigen binding domain having an amino acid sequence having at least 98% identity to an amino acid sequence of SEQ ID NO: 51, SEQ ID NO: 52, SEQ ID NO: 55, or SEQ ID NO: 56.
   100. The viral particle of any one of embodiments 97-99, wherein the chimeric antigen receptor comprises an antigen binding domain comprising an amino acid sequence of SEQ ID NO: 51 or SEQ ID NO: 52.
   101. A method of treating cancer in a subject in need thereof, the method comprising administering to the subject a viral particle of any one of embodiments 97-100.
   102. The method of embodiment 101, wherein the cancer is a cancer as provided for herein, such as a T cell or B cell disorder.

The following examples are illustrative, but not limiting, of the compounds, compositions and methods described herein. Other suitable modifications and adaptations known to those skilled in the art are within the scope of the following embodiments.

### EXAMPLES

### Example 1: Spring Viremia of Carp Virus G Protein can Facilitate Cell Specific Fusion

Methods

Plasmids/Sequences. All rhabdovirus GP sequences were codon optimized and synthesized by Genscript. Sequences are shown below, with accession numbers found in the following table, which are hereby incorporated by reference in its entirety, Table 1:

TABLE 1
Rhadovirus glycoprotein sequences used.

| Rhabdovirus | Genbank Accession |
|---|---|
| Spodoptera frugiperda rhabdovirus isolate Sf - G | KF947078.1 |
| Drosophila obscura sigma virus 10A - G | GQ410979.1 |
| Wuhan insect virus 7 - G | YP_009301742.1 |
| Perch rhabdovirus - G | YP_007641366.1 |
| Spring viremia of carp virus - G | CAA85735.1 |

The targeting moiety comprises an anti-CD7 antibody (clone MT701, in single chain format) anchored to the membrane via an IgG based Fc stalk, which comprises a CD28 transmembrane domain.

Cells. HEK293T cells were grown in DMEM with 10% FBS. SupT1 cells were maintained in RPMI media with 10% FBS. Human PBMCs were purchased from AllCells and cultured in X-Vivo 10 (Lonza) supplemented with 20 ng/mL IL-2 (Peprotech). PBMCs were activated 48 hours prior to transduction using anti-CD3/CD28 Dynabeads (Cell Therapy Systems).

Generation of lentiviral particles. The recombinant lentiviral particles co-expressing rhabdovirus-G glycoproteins and binder molecules were generated by plasmid transection into HEK293T cells using Lipofectamine 3000 (ThermoFisher Scientific). A total 5 plasmids were transfected: (1) plasmid expressing the rhabdovirus G glycoprotein, (2) plasmid expressing the binder protein (where indicated), (3) plasmid expressing the lentiviral transfer genome encoding for CAR20, (4) plasmid expressing gag-pol, and (5) plasmid expressing rev. Transfected cell supernatant was harvested 48 hours later. Virus in the cell supernatant was concentrated by centrifugation through a sucrose cushion and resuspended in x-vivo medium. Lentiviral particle titer was determined using the Lenti-X p24 Rapid Titer Kit (Takara Bio, San Jose, CA).

Lentivirus transduction assay. A series of 5-fold dilutions (in cell culture media) of the concentrated lentivirus was performed and used to infect SupT1 and activated human PBMCs. Media was replaced 6 hours later, and the transduced cells were analyzed by flow cytometry on days 4 and 7 after transduction. Cells were stained with a viability stain, an anti-CD7 antibody to detect CD7 positive cells, and an anti-CAR antibody to detect CAR20 expression as a measure of transduction efficiency.

Results:

SVCV-G mediates fusion into human cells. Most non-mammalian rhabdovirus G proteins failed to mediate transduction in human cells, with the exception of Spring viremia of carp virus G (SVCV-G). This glycoprotein did not target SupT1 or human PBMC cells on its own as transduction was very inefficient (˜1 log lower than blinded VSV-G I182E) in the absence of the CD7 binder. Targeted transduction mediated by the CD7 binder reached comparable titers to retargeted VSV-G I182E.

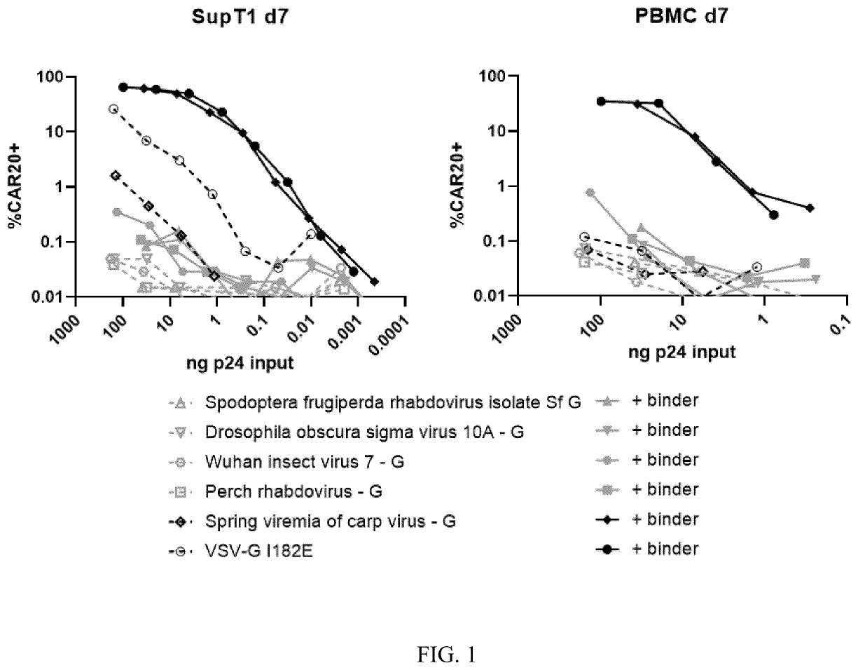

This surprising and unexpected result demonstrates that a viral structural protein that cannot infect an immune cell on its own can be used to infect a cell when the virus also expresses a targeting moiety, such as one that comprises an anti-CD7 scFv. This result could not have been predicted and is shown not to be an effect of the anti-CD7 scFv on its own as the presence of the anti-CD7 scFv targeting moiety was not sufficient to induce infections utilizing other non-human rhabdovirus GP proteins. The data is illustrated FIG. 1.

### Example 2: SVCV-G Pseudotyped Lentivirus Harboring CD7 Binder Transduces PBMCs

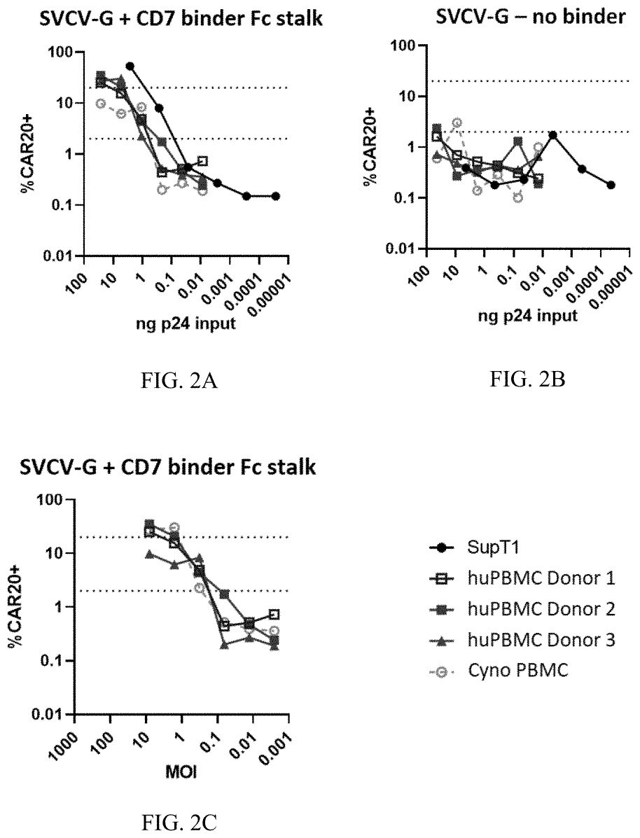

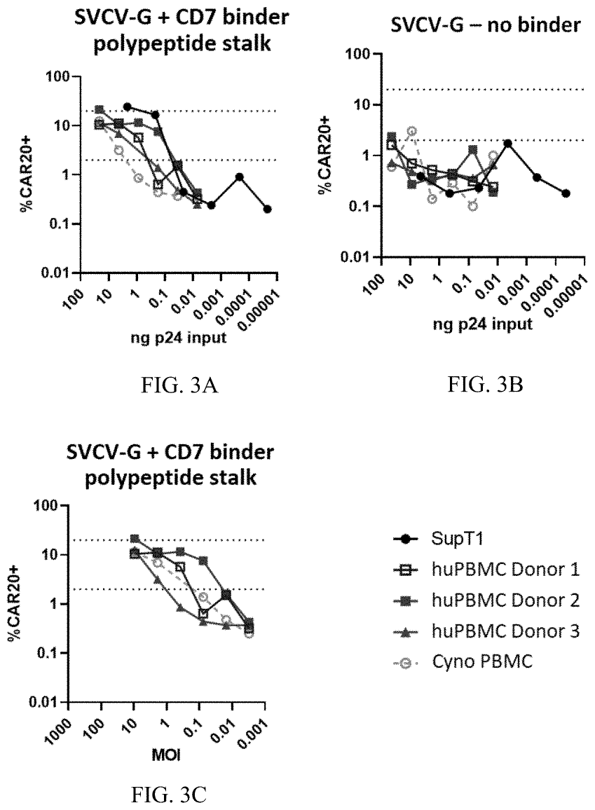

Human PBMC cells were maintained in X-Vivo10 media with 5% human serum and 20 ng/mL of human IL-2. Non-human primate cells were maintained in RPMI media with 10% FBS, 1% Pen/Strep with 1 mM Sodium Pyruvate and 100 units/mL hIL-2. Concentrated lentivirus was used to infect human and non-human primate cells. Media was replaced 24 hours later, and the transduced cells were analyzed by flow cytometry on day 7 after transduction to determine CAR transduction. Two separate binder constructs were assessed, a CD7 binder attached to the viral surface through a Fc domain, and a CD7 binder attached to the viral surface through a polypeptide stalk, such as provided for herein. For the CD7 binder attached to the viral surface through a Fc domain, CAR expression was observed in all human and non-human primate PBMCs assessed (FIG. 2A) compared to the binder alone (FIG. 2B) which shows no transduction. Data are also presented as percent CAR expression vs multiplicity of infection (FIG. 2C). For the CD7 binder attached to the viral surface through a polypeptide stalk, CAR expression was also observed in all human and non-human primate PBMCs assessed (FIG. 3A) compared to the binder alone (FIG. 3B) which shows no transduction. Data are also presented as percent CAR expression vs multiplicity of infection (FIG. 3C).

The data from the present example illustrates that the SVCV-G pseudotyped lentiviruses are able to successfully transduce PBMCs while utilizing binder constructs with variable viral attachment sequences. This demonstrates the utility of SVCV-G to pseudotype a variety of viral constructs.

### Example 3: SVCV-G Pseudotyped Lentivirus Harboring CD7 Binder Exhibits Minimal Off Target Transduction

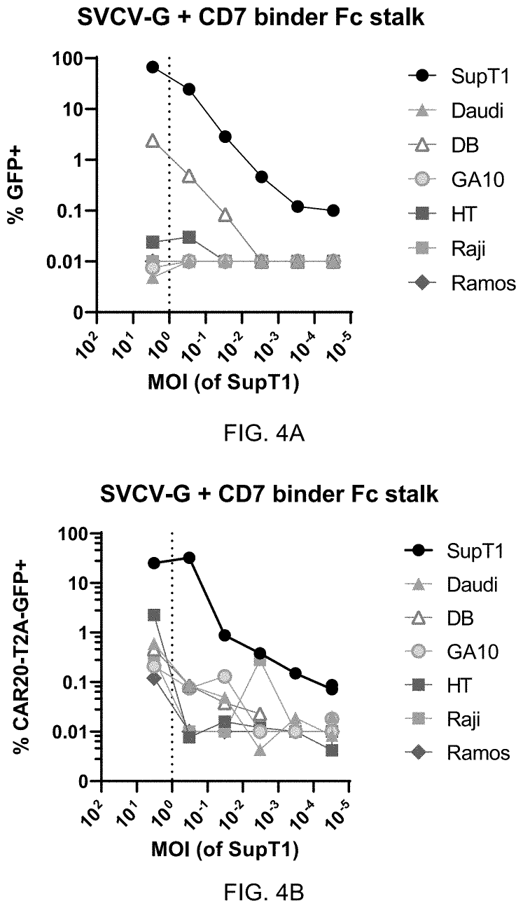

The off target transduction of B-cells via SVCV-G pseudotyped lentiviral constructs utilizing CD7 binder attached to the viral surface through a Fc domain was assessed in several B-cell populations. B-cells cells were maintained in X-Vivo10 media with 5% human serum and 20 ng/mL of human IL-2. Non-human primate cells were maintained in RPMI media with 10% FBS, 1% Pen/Strep with 1 mM Sodium Pyruvate and 100 units/mL hIL-2. Concentrated lentivirus was used to infect the various B-cell populations. Media was replaced 24 hours later, and the transduced cells were analyzed by flow cytometry on day 5 after transduction to determine GFP transduction. As a control, SupT1 cells were also infected with the same lentiviral constructs. None of the B-cell populations assessed demonstrated any significant level of GFP expression (FIG. 4A). The percent of GFP positive cells only exceeded 1% in line DB at the highest concentration of lentivirus utilized.

Lentiviral constructs delivering a CAR20-T2A-GFP transgene were also assessed in a similar manner (FIG. 4B). SVCV-G pseudotyped lentiviruses exhibited minimal off target transduction of B-cells in five of the six cell populations assessed. The percent of CAR-T2A-GFP positive cells only exceeded 1% in line HT at the highest concentration of lentivirus utilized.

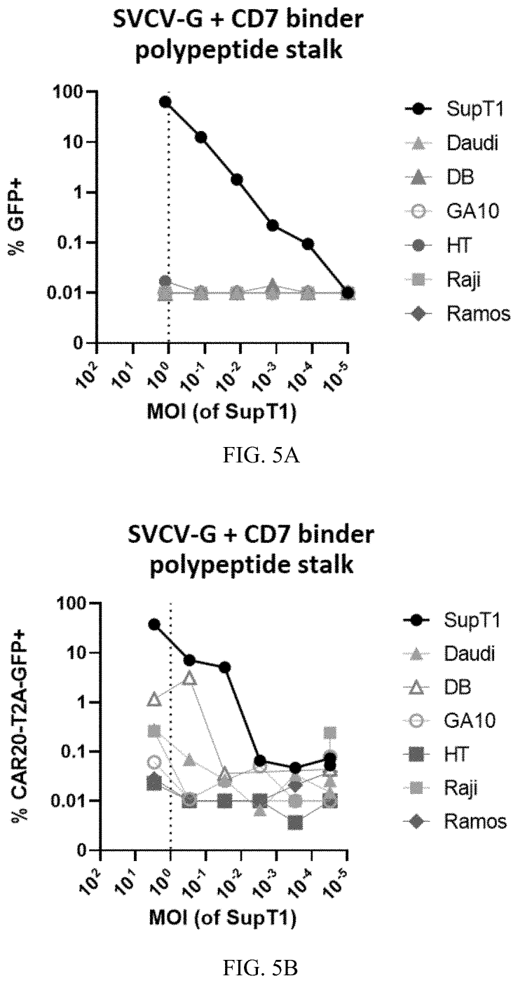

The off target transduction of B-cells via SVCV-G pseudotyped lentiviral constructs utilizing CD7 binder attached to the viral surface through a polypeptide stalk as provided for herein was also assessed. For GFP transduction experiments, none of the B-cell populations assessed demonstrated any significant level of GFP expression (FIG. 5A). For CAR20-T2A-GFP transduction experiments, SVCV-G pseudotyped lentiviruses exhibited minimal off target transduction of B-cells in five of the six cell populations assessed (FIG. 5B). The percent of CAR-T2A-GFP positive cells did not exceed 1% in the Daudi, GA10, HT, Raji, or Ramos cell lines at any of the lentiviral concentrations utilized. Transduction of the B-cell cell line DB did rise above 1% positive at two of the higher lentiviral concentrations utilized, but still remained less than the control SupT1 cell line.

The data of the present example illustrates that SVCV-G pseudotyped lentiviral constructs exhibit minimal off target transduction of B-cells, and delivery of a CAR20 transgene instead of a GFP transgene does not significantly increase off target transduction in B-cells.

The specification also makes reference to various sequences, such as those provided herein and below.

Spring Viremia of Carp Virus-G (SEQ ID NO: 1-with
leader sequence):
MSIISYIAFLLLIDSNLGIPIFVPSGRNISWQPVIQPFDYQCPIHGNLP
NTMGLSATKLTIKSPSVFSTDKVSGWICHAAEWKTTCDYRWYGPQYITH
SIHPISPTIDECRRIIQRIASGTDEDLGFPPQSCGWASVTTVSNTNYRV
VPHSVHLEPYGGHWIDHEFNGGECREKVCEMKGNHSIWITEETVQHECA
KHIEEVEGIMYGNVPRGDVMYANNFIIDRHHRVYRFGGSCQMKFCNKDG
IKFARGDWVEKTAGTLTTIHDNVPKCVDGTLVSGHRPGLDLIDTVFNLE
NVVEYTLCEGTKRKINKQEKLTSVDLSYLAPRIGGFGSVFRVRNGTLER
GSTTYIRIEVEGPIVDSLNGTDPRTNASRVFWDDWELDGNIYQGFNGVY
KGKDGKIHIPLNMIESGIIDDELQHAFQADIIPHPHYDDDEIREDDIFF
DNTGENGNPVDAVVEWVSGWGTSLKFFGMTLVALILIFLLIRCCVACTY
LMKRSKRPATESHEMRSLV
Spring Viremia of Carp Virus-G (SEQ ID NO: 2-
without leader sequence):
IPIFVPSGRNISWQPVIQPFDYQCPIHGNLPNTMGLSATKLTIKSPSVF
STDKVSGWICHAAEWKTTCDYRWYGPQYITHSIHPISPTIDECRRIIQR
IASGTDEDLGFPPQSCGWASVTTVSNTNYRVVPHSVHLEPYGGHWIDHE
FNGGECREKVCEMKGNHSIWITEETVQHECAKHIEEVEGIMYGNVPRGD
VMYANNFIIDRHHRVYRFGGSCQMKFCNKDGIKFARGDWVEKTAGTLTT
IHDNVPKCVDGTLVSGHRPGLDLIDTVFNLENVVEYTLCEGTKRKINKQ
EKLTSVDLSYLAPRIGGFGSVFRVRNGTLERGSTTYIRIEVEGPIVDSL
NGTDPRTNASRVFWDDWELDGNIYQGFNGVYKGKDGKIHIPLNMIESGI
IDDELQHAFQADIIPHPHYDDDEIREDDIFFDNTGENGNPVDAVVEWVS
GWGTSLKFFGMTLVALILIFLLIRCCVACTYLMKRSKRPATESHEMRSL
V
VSV-G I182E (SEQ ID NO: 3):
MKCLLYLAFLFIGVNCKFTIVFPHNQKGNWKNVPSNYHYCPSSSDLNWH
NDLIGTALQVKMPKSHKAIQADGWMCHASKWVTTCDFRWYGPKYITHSI
RSFTPSVEQCKESIEQTKQGTWLNPGFPPQSCGYATVTDAEAVIVQVTP
HHVLVDEYTGEWVDSQFINGKCSNYICPTVHNSTTWHSDYKVKGLCDSN
LESMDITFFSEDGELSSLGKEGTGFRSNYFAYETGGKACKMQYCKHWGV
RLPSGVWFEMADKDLFAAARFPECPEGSSISAPSQTSVDVSLIQDVERI
LDYSLCQETWSKIRAGLPISPVDLSYLAPKNPGTGPAFTIINGTLKYFE
TRYIRVDIAAPILSRMVGMISGTTTERELWDDWAPYEDVEIGPNGVLRT
SSGYKFPLYMIGHGMLDSDLHLSSKAQVFEHPHIQDAASQLPDDESLFF
GDTGLSKNPIELVEGWFSSWKSSIASFFFIIGLIIGLFLVLRVGIHLCI
KLKHTKKRQIYTDIEMNRLGK
Perch rhabdovirus-G (SEQ ID NO: 4):
MNTLIKILLIIIILREARSHIVLVPLDLGDWRTTEADQLDCPMHGDLSN
QGTQAIELEYHTASWGLKNNIAGSLCVTAKWSITCDYRWYGSKYISTVI
EYLPTTPEMCKEAKRASDRGESLAPHFPTENCGWNNVLTESQEFTTLTS
HPVKLDAYSFILIDSMFEGGRCQSKECPVVFHQGMWIADQEAFGFCKDL
DKHRGLLFKTGLRNSLGEIVRQEWNLNSVFQPEIGREKHFKGACKMSYC
GNSGVRFSDREWFQLGTPSDNGIKKIIEGLPECGEDNLIHSHDTSNTLK
ELAEHVDEIALNAICLQEVRRARDTKAVSDWLLSMMSPFSEGIGKVYRI
HKGRLESTVGFYRKVVLEGDGTPERLGVGLDKKPVSWDQFVVKINDTRI
QSMFNGNTVVNGKIKWVKNVLGAHILDEISALEFDVPLIPHPHLDGLKF
NESHTISSHHPNGKGVNFVESVTHWAGGLWESIGSSAVIIVVLLICAFV
AVKFCQRLIPSRRPPTRESSENVFMLRTV
Drosophila obscura sigma virus 10-A-G
(SEQ ID NO: 5):
MKKTTEFTWPLKMYSSKFLLVTLSILVPRVISLFLPDYNPELFKPAVIH
KLTCPNLSATSQINKYASMDTIYVNLGRPKPNFKVTVEGYLCTKIVLQT
VCEAFLFSANEVNHIRKEAPIDKSECDSAIYRFIKGDVIDETFLPDYFS
GYKRTVRQRIYIKVITHEVSYDPYLEKYVDSWFPGGATPYNYSTTIHDS
TLWKMKGQKPPCTDFETINGEYTVLPDPKDLEHPLRFIWALGIKEKSYK
NSCVVKFCGRPGILFEDGEWFSISTPESPTNYDVFITNLPACNSSASIK
TRTVEQEMDAEFSNEYQLNLRFWCMEVLQGLISGSILPQYKLAFLTQNS
PGLGNVYKILNGTLMVTVGHYKKVKLNQLDSLHEIGADDAGNPVYVTDT
QLTPGPIPDSYEWINGLVFSDKRWSIPLVDLVRNNLDNLLTMPLKLQAI
EHSAASFMSASSSHLIEGDVITQYGGPTSKHDNWFVNYTTKSWEWLHWI
VYIGVIVGGIFVLIILEKLGCYKIVYYLLKKLCQSKSSKKTKQPKVEYK
KASSNQAQQIESRHEVIPQEDSVIVSLDRFDW
Spodoptera frugiperda virus-G (SEQ ID NO: 6):
MVFLSLSTIIFILSLRAVTCSNPLSYPNGILINNSTHNHPLSDFYIFYE
NSSLTYTQFPVAPDCSSILDTRDEQYPTTVTLWKVDQESQAEWGLLLWQ
ERIDTTCSWNFWGNYKGSIVSKSSVPLKDIPSGSARNGYWALSNDEVQE
IDHVPYNLRYYCYWCRNEYPGSFYMRYVKKVRIIRNPDGSIKTPRGSWV
HELDNLWGDQMRYLVIRRFGGESSCPLKIYDVRAGVLSKSRSNFILVSL
PSLNLQFSVSLESTETKCSFGDKTYDIVQSMGGYLLSIDIGNANWRGPW
DPTPQHPGRERRSIMEFPDQTSFRYNQFINYHSSPRHKRHDQEFEFPLS
LKSSYDYAQFRYEQNFIIRQINKNFGLLQKSICDIQFSKWQNLSPPNLA
MKIAHYVTGSIHSIGGVHHGSYSIQRTEKSITKVNLVFPIVIVHGMYKC
QREPSKEVVWAEPVTGILFKSPIPTHFSLSSSWLPGVNGSSIVPLTGQI
LLPEITMDHLEVVQQVEAKMVKSMYTNVELFGSTEEFQRYQTQGITSDE
QSNTVNPWIGLLIHGGVSIATGILVALLIPSILKLFRHIIEKGEASLEE
RLHLRETSRKEFVKVRGKPWGV
Wuhan Insect virus 7-G (SEQ ID NO: 7):
MFPSIFCLMLLVMTSSVGQFILPIKEITPPRPINEHHLSCRLGPGALYP
TTSSSVVVRSIHPDSNPVPIQGYLCSKKEYTLTCSENFLWMETVKKDSR
SVPVTPSECVDAVNSWLQDKLDKDTQLTKDCGWMSTNTVKKEIFDIQGK
HVRLNPYEMEYIDEILHRGKCSSKICQTKYADMLWVSEGDLNQTCPKMI
GADVRMSLGDQKTPSSVILHSNYVPGVSLEGACRDFSFCGHRGMVLRTG
HFVVVSSDFPSTIVKMYNLFQVCQKDLTIVSDNMNSEITSSKLDLMKIF
LLDKCQQVVARIKGGLSVTRYDLGFLSPQISGVGSGYVASNRSVLVGQY
KYMLVHSYVANCHSCRSCVINAVTDEGVIPVHLTPDQCPCANFNGCYLP
NGIKVYKDKVYNPLLDIDEEFYHQMTEVELNPNYISHPKDVKETFLSLP
ATEEVEQHHGRTLDSVVGSLVPDFSFSIWPYFVGLGALVIIIIILWRRR
SYKPPAEAPIRDFPVIYTPQSSFGPIAQIRWN

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

1-102. (canceled)

103. A viral particle comprising a targeting moiety and a polypeptide comprising a viral structural protein comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to an amino acid sequence of SEQ ID NO: 1 or SEQ ID NO: 2.

104. The viral particle of claim 103, wherein the viral structural protein comprises a polypeptide comprising the amino acid sequence of SEQ ID NO: 1 or SEQ ID NO: 2.

105. The viral particle of claim 103, wherein the viral particle is a pseudotyped lentivirus.

106. The viral particle of claim 103, wherein the viral particle further comprises a nucleic acid molecule encoding a heterologous molecule of interest.

107. The viral particle of claim 106, wherein the heterologous molecule of interest is an siRNA, an shRNA, a non-coding RNA (e.g. a guide RNA for a CRISPR system), a peptide, a polypeptide, a protein, a viral payload, a viral genome, or a combination thereof.

108. The viral particle of claim 106, wherein the heterologous molecule of interest is a chimeric antigen receptor (“CAR”).

109. The viral particle of claim 108, wherein the CAR comprises an antigen binding domain having an amino acid sequence having at least 85%. 86%, 87%, 88%, 89%, 90%, 91%, 92%, 93%, 94%, 95%, 96%, 97%, 98%, 99%, or 100% identity to SEQ ID NO: 51 or SEQ ID NO: 52.

110. The viral particle of claim 108, wherein the CAR comprises an antigen binding domain having an amino acid sequence having the amino acid sequence of SEQ ID NO: 51 or SEQ ID NO: 52.

111. The viral particle of claim 103, wherein the targeting moiety binds to an immune cell, such as a T cell, B cell; NK cell, dendritic cell, neutrophils, macrophages, a cancer cell; or, for example, CD3+ T cell; CD4+ T cell; CD7+ T cell, CD8+ T cell; CD19+ B cell; CD19+ cancer cell; CD20+ B cell; “CD20+ cancer cell, CD30+ lung epithelial cell; CD34+ haematopoietic stem cell; CD105+ endothelial cell; CD105+ haematopoietic stem cell; CD117+ haematopoietic stem cell; CD133+ cancer cell; EpCAM+ cancer cell; GluA2+ neuron; GluA4+ neuron; Haematopoietic stem cell; Hepatocyte; Her2/Neu+ cancer cell; NKG2D+ natural killer cell; SLC1A3+ astrocyte; SLC7A10+ adipocyte.

112. The viral particle of claim 103, wherein the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsg1); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-1a); Carbonic anhydrase IX (CA1X); Carcinoembryonic antigen (CEA); CCCTC-Binding Factor (Zinc Finger Protein)-Like (BORIS or Brother of the Regulator of lmprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLVl-Tax; Human papilloma virus E6 (HPV E6); Human papilloma virus E7 (HPV E7); Human Telomerase reverse transcriptase (hTERT); IgE; IL13Ra2; ILl lRa; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-1 (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAP1; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1).

113. The viral particle of claim 103, wherein the targeting moiety is attached to the viral surface through:
an IgG Fc stalk;
a polypeptide stalk;
an envelope glycoprotein G or H of a virus of the Paramyxoviridae family, such as a morbillivirus, such as Measles virus, or a henipavirus, such as Nipah virus, Cedar virus, or Hendra virus;
a glycoprotein of a virus of the Rhabdoviridae family, such as a vesicular stomatitis New Jersey virus, a vesicular stomatitis Indiana virus, a vesicular stomatitis Alagoas virus, a vesicular stromatitis Maraba virus, a vesicular stomatitis Carajas virus, Parainfluenza virus, Spodoptera frugiperda rhabdovirus isolate Sf G, Drosophila obscura sigmavirus 10A, Wuhan insect virus 7, Perch virus, or Spring viremia of carp virus;
a glycoprotein of a virus of the Filoviridae family, such as Ebola virus; or
a glycoprotein of a virus of the Arenaviridae family, such as Machupo virus.

114. The viral particle of claim 103, wherein viral particle comprises an IgG Fc stalk linked to a targeting moiety, wherein, for example, the IgG Fc stalk is attached to the viral surface.

115. The viral particle of claim 103, wherein the targeting moiety binds to CD7.

116. The viral particle of claim 103, wherein the targeting moiety binds to CD8.

117. A method of making the viral particle of claim 103, the method comprising transfecting or transducing a viral packaging cell with a nucleic acid molecule encoding the polypeptide and a nucleic acid molecule encoding the targeting moiety under conditions sufficient to produce the viral particle.

118. A method of delivering a heterologous molecule to a target cell, the method comprising contacting the cell or administering to a subject comprising the target cell with a viral particle of claim 106.

119. A method of treating cancer in a subject, the method comprising administering to the subject a viral particle of claim 106.

120. A viral particle comprising a targeting moiety, a polypeptide comprising a viral structural protein, and a nucleic acid molecule encoding a heterologous molecule of interest, wherein:
a) the targeting moiety comprises an amino acid sequence having at least 90% identity to an amino acid sequence of SEQ ID NO: 28, SEQ ID NO: 29, SEQ ID NO: 46, or SEQ ID NO: 47;
wherein the polypeptide comprising a viral structural protein comprises an amino acid sequence having at least 90% identity to an amino acid sequence of SEQ ID NO: 1 or SEQ ID NO: 2;
wherein the nucleic acid molecule encoding a heterologous molecule of interest encodes for a chimeric antigen receptor; and
wherein the chimeric antigen receptor comprises an antigen binding domain having an amino acid sequence having at least 90% identity to an amino acid sequence of SEQ ID NO: 51, SEQ ID NO: 52, SEQ ID NO: 55, or SEQ ID NO: 56;
b) the targeting moiety comprises an amino acid sequence having at least 95% identity to an amino acid sequence of SEQ ID NO: 28, SEQ ID NO: 29, SEQ ID NO: 46, or SEQ ID NO: 47;
wherein the polypeptide comprising a viral structural protein comprises an amino acid sequence having at least 95% identity to an amino acid sequence of SEQ ID NO: 1 or SEQ ID NO: 2;
wherein the nucleic acid molecule encoding a heterologous molecule of interest encodes for a chimeric antigen receptor; and
wherein the chimeric antigen receptor comprises an antigen binding domain having an amino acid sequence having at least 95% identity to an amino acid sequence of SEQ ID NO: 51, SEQ ID NO: 52, SEQ ID NO: 55, or SEQ ID NO: 56; or
c.) wherein the targeting moiety comprises an amino acid sequence having at least 98% identity to an amino acid sequence of SEQ ID NO: 28, SEQ ID NO: 29, SEQ ID NO: 46, or SEQ ID NO: 47;
wherein the polypeptide comprising a viral structural protein comprises an amino acid sequence having at least 98% identity to an amino acid sequence of SEQ ID NO: 1 or SEQ ID NO: 2;
wherein the nucleic acid molecule encoding a heterologous molecule of interest encodes for a chimeric antigen receptor; and
wherein the chimeric antigen receptor comprises an antigen binding domain having an amino acid sequence having at least 98% identity to an amino acid sequence of SEQ ID NO: 51, SEQ ID NO: 52, SEQ ID NO: 55, or SEQ ID NO: 56.

121. A method of treating cancer in a subject in need thereof, the method comprising administering to the subject a viral particle of claim 120.

122. The method of claim 121, wherein the cancer is a cancer as provided for herein, such as a T cell or B cell disorder.
